SEQUENCE LISTING (26 sequences)

SEQ ID NO: 1 - protein, 426 aa, Homo sapiens
MFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 2 - DNA, 3340 bp, Homo sapiens
  source 1..3340: mol_type = other DNA
attgcctgcttctccccacccccaaattaagttgcttagcaagggggaaagaggctttttccttcttccaacagcccagccgaacgcctttcgttttttgcccccgcggaccttccatgtaggaagccgaggctggcgagcccgacattcgggagccactgcgggggggcctctttttggggaggcgccgacgggggcaggctcggccgtccccagggaagcggcggccgggttcctccggggcgcgccggggccggagagccgcgcagggcgcgggccgcgcggggtggggcagccggagcgcaggcccccgatccccggcgggcgcccccgggcccccgcgcgcgccccggcctccgggagactggcgcatgccacggagcgcccctcgggccgccgccgctcctgcccgggcccctgctgctgctgctgtcgcctgcgcctgctgccccaactcggcgcccgacttcttcatggtgtgcggaggtcatgttcgctccttagcaggcaaacgacttttctcctcgcctcctcgccccgcatgttcaggaccaaacgatctgcgctcgtccggcgtctctggaggagccgtgcgcccggcggcgaggacgaggaggagggcgcagggggaggtggaggaggaggcgagctgcggggagaaggggcgacggacagccgagcgcatggggccggtggcggcggcccgggcagggctggatgctgcctgggcaaggcggtgcgaggtgccaaaggtcaccaccatccccaccgccagccgcgggcgccggcgcggccgggggcgccgaggcggatctgaaggcgctcacgcactcggtgctcaagaaactgaaggagcggcagctggagctgctgctccaggccgtggagtcccgcggcgggacgcgcaccgcgtgcctcctgctgcccggccgcctggactgcaggctgggcccgggggcgcccgccggcgcgcagcctgcgcagccgccctcgtcctactcgctccccctcctgctgtgcaaagtgttcaggtggccggatctcaggcattcctcggaagtcaagaggctgtgttgctgtgaatcttacgggaagatcaaccccgagctggtgtgctgcaacccccatcaccttagccgactctgcgaactagagtctcccccccctccttactccagatacccgatggattttctcaaaccaactgcagactgtccagatgctgtgccttcctccgctgaaacagggggaacgaattatctggcccctggggggctttcagattcccaacttcttctggagcctggggatcggtcacactggtgcgtggtggcatactgggaggagaagacgagagtggggaggctctactgtgtccaggagccctctctggatatcttctatgatctacctcaggggaatggcttttgcctcggacagctcaattcggacaacaagagtcagctggtgcagaaggtgcggagcaaaatcggctgcggcatccagctgacgcgggaggtggatggtgtgtgggtgtacaaccgcagcagttaccccatcttcatcaagtccgccacactggacaacccggactccaggacgctgttggtacacaaggtgttccccggtttctccatcaaggctttcgactacgagaaggcgtacagcctgcagcggcccaatgaccacgagtttatgcagcagccgtggacgggctttaccgtgcagatcagctttgtgaagggctggggccagtgctacacccgccagttcatcagcagctgcccgtgctggctagaggtcatcttcaacagccggtagccgcgtgcggaggggacagagcgtgagctgagcaggccacacttcaaactactttgctgctaatattttcctcctgagtgcttgcttttcatgcaaactctttggtcgttttttttttgtttgttggttggttttcttcttctcgtcctcgtttgtgttctgttttgtttcgctctttgagaaatagcttatgaaaagaattgttgggggtttttttggaagaaggggcaggtatgatcggcaggacaccctgataggaagaggggaagcagaaatccaagcaccaccaaacacagtgtatgaaggggggcggtcatcatttcacttgtcaggagtgtgtgtgagtgtgagtgtgcggctgtgtgtgcacgcgtgtgcaggagcggcagatggggagacaacgtgctctttgttttgtgtctcttatggatgtccccagcagagaggtttgcagtcccaagcggtgtctctcctgccccttggacacgctcagtggggcagaggcagtacctgggcaagctggcggctggggtcccagcagctgccaggagcacggctctgtccccagcctgggaaagcccctgcccctcctctccctcatcaaggacacgggcctgtccacaggcttctgagcagcgagcctgctagtggccgaaccagaaccaattattttcatccttgtcttattcccttcctgccagcccctgccattgtagcgtctttcttttttggccatctgctcctggatctccctgagatgggcttcccaagggctgccggggcagccccctcacagtattgctcacccagtgccctctcccctcagcctctcccctgcctgccctggtgacatcaggtttttcccggacttagaaaaccagctcagcactgcctgctcccatcctgtgtgttaagctctgctattaggccagcaagcggggatgtccctgggagggacatgcttagcagtccccttccctccaagaaggatttggtccgtcataacccaaggtaccatcctaggctgacacctaactcttctttcatttcttctacaactcatacactcgtatgatacttcgacactgttcttagctcaatgagcatgtttagactttaacataagctatttttctaactacaaaggtttaaatgaacaagagaagcattctcattggaaatttagcattgtagtgctttgagagagaaaggactcctgaaaaaaaacctgagatttattaaagaaaaaaatgtattttatgttatatataaatatattattacttgtaaatataaagacgttttataagcatcattatttatgtattgtgcaatgtgtataaacaagaaaaataaagaaaagatgcactttgctttaatataaatgcaaataacaaatgccaaattaaaaaagataaacacaagattggtgtttttttctatgggtgttatcacctagctgaatgtttttctaaaggagtttatgttccattaaacgatttttaaaatgtacacttga

SEQ ID NO: 3 - protein, 224 aa, Homo sapiens
ELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 4 - protein, 156 aa, Homo sapiens
TRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 5 - protein, 15 aa, Homo sapiens
ELESPPPPYSRYPMD

SEQ ID NO: 6 - protein, 11 aa, synthetic construct
YGRKKRRQRRR

SEQ ID NO: 7 - protein, 13 aa, synthetic construct
GRKKRRQRRRPPQ

SEQ ID NO: 8 - protein, 9 aa, synthetic construct
RKKRRQRRR

SEQ ID NO: 9 - protein, 10 aa, synthetic construct
GRKKRRQRRR

SEQ ID NO: 10 - protein, 16 aa, synthetic construct
RQIKIWFQNRRMKWKK

SEQ ID NO: 11 - protein, 24 aa, synthetic construct
LGTYTQDFNKFHTFPQTAIGVGAP

SEQ ID NO: 12 - protein, 21 aa, synthetic construct
KETWWETWWTEWSQPKKKRKV

SEQ ID NO: 13 - protein, 18 aa, synthetic construct
KLALKLALKALKAALKLA

SEQ ID NO: 14 - protein, 27 aa, synthetic construct
GWTLNSAGYLLGKINLKALAALAKKIL

SEQ ID NO: 15 - protein, 18 aa, synthetic construct
LLIILRRRIRKQAHAHSK

SEQ ID NO: 16 - protein, 28 aa, synthetic construct
LSTAADMQGVVTDGMASGLDKDYLKPDD

SEQ ID NO: 17 - protein, 34 aa, synthetic construct
NAATATRGRSAASRPTQRPRAPARSASRPRRPVQ

SEQ ID NO: 18 - protein, 22 aa, synthetic construct
MVRRFLVTLRIRRACGPPRVRV

SEQ ID NO: 19 - protein, 26 aa, synthetic construct
DPKGDPKGVTVTVTVTVTGKGDPKPD

SEQ ID NO: 20 - protein, 17 aa, synthetic construct
CSIPPEVKFNKPFVYLI

SEQ ID NO: 21 - protein, 10 aa, synthetic construct
PIEVCMYREP

SEQ ID NO: 22 - protein, 15 aa, synthetic construct
SDLWEMMMVSLACQY

SEQ ID NO: 23 - protein, 6 aa, synthetic construct
PFVYLI

SEQ ID NO: 24 - protein, 20 aa, synthetic construct
CVKRGLKLRHVRPRVTRMDV

SEQ ID NO: 25 - protein, 9 aa, synthetic construct
YPYDVPDYA

SEQ ID NO: 26 - protein, 5 aa, synthetic construct
GPLGS

(19) United States
(12) Patent Application Publication
(10) Pub. No.: US 2024/0245751 A1
(43) Pub. Date: Jul. 25, 2024

(54) SMAD7 POLYPEPTIDE FORMULATIONS

(71) Applicant: THE REGENTS OF THE UNIVERSITY OF COLORADO, A BODY CORPORATE, Denver, CO (US)

(72) Inventors: Xiao-Jing WANG, West Sacramento, CA (US); Christian D. YOUNG, Denver, CO (US)

(21) Appl. No.: 18/599,948

(22) Filed: Mar. 8, 2024

Related U.S. Application Data
(63) Continuation of application No. PCT/US2022/076242, filed on Sep. 9, 2022.
(60) Provisional application No. 63/244,338, filed on Sep. 15, 2021.

(51) Int. Cl. A61K 38/17; A61K 9/06; A61K 47/38; A61P 17/02; A61P 29/00

(52) U.S. Cl. CPC A61K 38/1709; A61K 9/06; A61K 47/38; A61P 17/02; A61P 29/00

(57) ABSTRACT

Embodiments of the instant disclosure generally relate to formulations for preserving, storing, administering and/or delivering Smad7 polypeptides or fragments thereof to a subject to reduce the risk of onset, or to prevent or treat a health condition. Certain embodiments relate to administering and/or delivering Smad7 formulations to treat a subject having adverse effects due to excessive inflammation, radiation, chemotherapy, other anti-tumor treatments, infection and/or necrosis. In some embodiments, a Smad7 polypeptide can include a Smad7 fragment as part of a fusion polypeptide formulated for topical or dermal delivery to a subject further including a gelling agent to improve efficacy of the formulation.

### PRIORITY

This International Application claims priority to U.S. Provisional Application No. 63/244,338 filed Sep. 15, 2021. This application is incorporated herein by reference in its entirety for all purposes.

### GOVERNMENT SUPPORT

This invention was made with government support under Grant Nos. R44DE024659, R44DE028718, and R44AR078669 awarded by the National Institutes of Health. The government has certain rights in this invention.

### STATEMENT REGARDING SEQUENCE LISTING

The instant application contains a Sequence Listing which has been submitted 9 Sep. 2022 as a .xml file named “108945-738927 CU5673H-PCT_Sequence Listing”, created on 6 Sep. 2022 and having a size of 35 kilobytes which is hereby incorporated by reference in its entirety for all purposes pursuant to 37 C.F.R. § 1.52(e)(5).

### FIELD

Embodiments of the instant disclosure generally relate to formulations and methods for making, preserving, storing, administering and/or delivering SMAD7 polypeptides or fusion molecules thereof to a subject to reduce the risk of onset, or to treat a health condition.

### BACKGROUND

Mothers against decapentaplegic homolog 7 or Smad7 is a protein encoded by the SMAD7 gene. Studies have demonstrated that Smad7 and active fragments thereof have been demonstrated of use in the treatment of certain health conditions. See for example, PCT/US11/52499; PCT/US14/22052. In order to more effectively and efficiently deliver these molecules systemically and/or locally to a subject in need, improved formulations are sought.

### SUMMARY

Embodiments of the present disclosure relate to compositions for preserving, storing, formulating, administering and/or delivering SMAD7 polypeptides to a subject. In some embodiments, formulations disclosed herein can be administered to a subject after the formulation is stored for a period of time before administration to the subject, to reduce the risk of onset, or treat a health condition (e.g. inflammatory condition). Certain embodiments relate to administering and/or delivering SMAD7 formulations to treat a subject having adverse effects including, but not limited to, at least one of excessive inflammation, side effects of radiation treatment, side effects of chemotherapy treatment, other anti-tumor treatment side effect and/or necrosis.

In some embodiments, formulations for administering and/or delivering SMAD7 polypeptides can be topical formulations of use to treat dermal and/or oral complications of a therapy or side effect of a health condition in a subject. In certain embodiments, the dermal and/or oral complication can be caused by chemotherapy and radiation therapy to the subject as a side effect of these therapies. In accordance with these embodiments, a topical formulation disclosed herein includes at least one recombinant human Smad7 polypeptide construct, a fragment thereof, or fusion polypeptide or fragment thereof and at least one gelling agent. In certain embodiments, the at least one gelling agent preserves or improves delivery of the recombinant human Smad7 polypeptide, a fragment thereof, or fusion polypeptide or fragment thereof by reducing degradation and/or facilitating delivery or prolonging exposure of the formulation to a dermal surface or oral surface of the subject to treat the subject.

Some embodiments of the present disclosure provide compositions that can include at least one gelling agent and at least one recombinant human Smad7, a fragment thereof, or fusion polypeptide thereof. In certain embodiments, compositions disclosed herein can further include one or more protein stabilizers, one or more surfactants, one or more preservatives, one or more antimicrobial agents, one or more salts or any combination thereof. In some embodiments, the at least one gelling agent can have an average molecular weight (MW) of about 25,000 to about 1,500,000 Daltons for use to prolong half-life and deliver Smad7 molecules disclosed herein.

In certain embodiments, the at least one gelling agent can be one or more celluloses or derivatives thereof. In accordance with these embodiments, the at least one gelling agent can include at least one cellulose agent or derivative thereof including, but not limited to, benzylcellulose (BC), ethylcellulose (EC), methylcellulose (MC), hydroxypropylmethylcellulose (HPMC), cthylhydroxyethylcellulose (EHEC), carboxymethylcellulose (CMC), hydroxypropylcellulose (HPC), hydroxyethylcellulose (HEC), hydroxyethylmethylcellulose (HEMC), sodium carboxymethylcellulose (CMCNa), cellulose acetate (CA), cellulose nitrate, cellulose sulphate, cellulose acetate phthalatc (CAP), hydroxybutylcellulose, hydroxybutylmethylcellulose, carboxypropyl methylcellulose, hydroxypropyl methylcellulose (HPMC), cellulose acetate butyrate (CAB), microcrystalline cellulose (MCC), hydroxypropylmethylcellulose phthalate (HPMCP), cellulose acetate trimelitate (CAT), hydroxypropylmethylcellulose acetate succinate, or a combination thereof. In certain embodiments, the at least one cellulose agent or derivative thereof can include, but is not limited to, HPC, HEC, HPMC or a combination thereof. In some embodiments, the at least one cellulose agent or derivative thereof disclosed herein can further include one or more protein stabilizers, one or more surfactants, one or more preservatives, one or more antimicrobial agents, one or more salts or any combination thereof in the presence of a SMAD7 polypeptide, recombinant or fusion polypeptide thereof or fragment thereof or fusion polypeptide thereof.

In some embodiments, compositions disclosed herein can further include one or more protein stabilizers. In accordance with these embodiments, one or more protein stabilizers can include, but are not limited to, succinic anhydride, albumin, sialic acid, creatinine, glycine, histidine or other amino acid capable of stabilizing proteins, niacinamide, sodium acetyltryptophonate, zinc oxide, sucrose, glucose, lactose, trehalose, sorbitol, mannitol or other saccharide or disaccharide, glycerol, polyethylene glycols, sodium caprylate, sodium saccharin, or any combination thereof.

In some embodiments, compositions disclosed herein can further include one or more surfactants. In accordance with these embodiments, one or more surfactants of use in compositions disclosed herein can include, but are not limited to, sodium lauryl sulfate, sodium decussate, Tween-20, Tween-60, Tween-80; triacetin, vitamin E TPGS, a phospholipid, a lecithin, a phosphatidyl choline, a phosphatidylethanolamine, a phosphatidylglycerol, sorbitan monooleate, polyoxyethylene sorbitan monooleate, a polysorbate, a polaxomer, bile salt, glyceryl monostearate, a copolymer of ethylene oxide and propylene oxide, polyoxyethylene hydrogenated castor oil, polyoxyethylene alkylether, octoxynol 10, octoxynol 40, or equivalent thereof or any combination thereof.

In some embodiments, compositions herein can further include one or more preservatives that can be benzalkonium chloride, chlorobutanol, thimerosol, chloroxylenol, chlorhexidine, phenoxyethanol, benzyl alcohol, phenethyl alcohol, polyquaterniaum-1, diazolidinyl urea, iodopropynyl butylcarbamate, chloromethylisotiazolinone, methylisothiazolinone, vitamin E, a vitamin E derivative, vitamin E acetate, vitamin C, butylated hydroxytoluene, butylparaben, ethylparaben, methylparaben, propylparaben, isobutylparaben, phenoxyethanol, ethylparaben, propylparaben, utylparaben, or any combination thereof. In some embodiments, compositions herein can be free of preservatives.

In some embodiments, compositions disclosed herein can include a recombinant human Smad7, fragment thereof, or fusion polypeptide thereof having at least 85% identity to Smad7 or a fragment or fusion polypeptide thereof. In other embodiments, compositions disclosed herein can include a recombinant human Smad7, fragment thereof, or fusion polypeptide thercof having at least 85% identity to an amino acid sequence represented by SEQ ID NO: 1. In other embodiments, compositions disclosed herein can include a recombinant human Smad7, fragment thereof, or fusion polypeptide thereof having at least 85% identity to amino acids 203-426 of a Smad7 polypeptide. In other embodiments, compositions disclosed herein can include a recombinant human Smad7, fragment thereof, or fusion polypeptide thereof having at least 85% identity to an amino acid sequence represented by SEQ ID NO: 3. In other embodiments, compositions disclosed herein can include a recombinant human Smad7, fragment thereof, or fusion polypeptide thereof having at least 85% identity to amino acids 259-426 of a Smad7 polypeptide represented by SEQ ID NO: 1. In yet other embodiments, compositions disclosed herein can include a recombinant human Smad7, fragment thereof, or fusion polypeptide thereof having at least 85% identity to an amino acid sequence represented by SEQ ID NO: 4. In other embodiments, compositions disclosed herein can include a recombinant human Smad7, fragment thereof, or fusion polypeptide thereof having at least 85% identity to amino acids 203-217 of a Smad7 polypeptide. In yet other embodiments, compositions disclosed herein can include a recombinant human Smad7, fragment thereof, or fusion polypeptide thereof having at least 85% identity to an amino acid sequence represented by SEQ ID NO: 5. In some embodiments, a fusion polypeptide disclosed herein can include Smad7 or fragment thereof and a second polypeptide or other molecule fused to the Smad7 or fragment thereof. In accordance with these embodiments, the second polypeptide or other molecule fused to the Smad7, or fragment thereof can promote delivery of a fusion construct to a targeted region within the host (e.g., intra-organ, intracellular or intra-nuclear etc.) or increase half-life of the fusion molecule. In certain embodiments, a fusion polypeptide disclosed herein can include at least one protein transduction domain. In some embodiments, a recombinant Smad7, fragment thereof, or fusion polypeptide thereof can further include at least one epitope tag and/or a protein transduction domain.

In some embodiments, formulations containing recombinant Smad7, a fragment thereof, or a fusion polypeptide thereof can further include a gelling agent as disclosed herein in the presence of at least one salt having a pH of about 6 to about 9 or about pH 7 to about 9. In certain embodiments, the salt concentration of formulations disclosed herein is about 100 mM up to about 2.5 M salt. In accordance with these embodiments, formulations containing Smad7, or a fragment thereof are formulated for delivery to a subject.

Some embodiments of the present disclosure provide methods of reducing the risk of onset, preventing, treating, and/or ameliorating one or more symptoms associated with a disease or a health condition, where the methods can include administering one or more of the compositions disclosed herein to a subject having or suspected of having the one or more symptoms associated with the disease or the health condition. In certain embodiments, the one or more symptoms associated with the disease, or the health condition include, but is not limited to, inflammation, necrosis, oral mucositis, psoriasis, or a combination thereof. In some embodiments, oral mucositis, radiodermatitis, atopic dermatitis, allergic dermatitis, psoriasis, gastrointestinal mucosal complications, chronic (or diabetic) wounds, traumatic or surgically induces wounds (e.g., excisions, acute wounds post-surgery or post trauma), scarring, autoimmune related oral ulcer, periodontal inflammation, or a combination thereof can be a secondary result from one or more cancer treatments or other health condition. In certain embodiments, the one or more cancer treatments that can cause side effects during and post treatment can include, but are not limited to, oral mucositis, psoriasis, excess inflammation, and side effects thereof, or a combination thereof. Agents capable of causing these side effects include, but are not limited to, chemotherapeutics, radiation, topical anti-tumor agents, other anti-tumor agents or a combination thereof.

In some embodiments, compositions disclosed herein can be formulated for drop-wise administration or topical administration to the dermis, mucosal membrane, skin, oral mucosa, or a combination thereof. In certain embodiments, compositions disclosed herein can be administered to a mammalian subject. In some embodiments, compositions disclosed herein can be administered directly to cells of a mammalian subject. In other embodiments, compositions disclosed herein can be administered to a subject and/or a cell of a subject wherein the subject has completed, is about to begin, or is in the process of at least one treatment for a disease or a health condition (e.g., requiring cell-damaging treatments having side effects such as excisionally-caused wounds or post-surgical wounds, oral mucositis, radiodermatitis, atopic dermatitis, psoriasis or similar).

Some embodiments of the present disclosure provide kits containing formulations of Smad7 constructs and at least one gelling agent of the disclosure and/or for practicing methods of the disclosure and at least one container. In some embodiments, kits disclosed herein can include any of the compositions described herein and at least one container. In some embodiments, kits disclosed herein can include at least one multi-use package. In some embodiments, a multi-use package included in kits disclosed herein can include a polypropylene tube or other pharmaceutically acceptable topical deliver container, a syringe, or a combination thereof.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A and 1B represent exemplary experiments of the instant disclosure illustrating generation and purification of a representative Smad7 fragment protein in accordance with certain embodiments disclosed herein.

FIG. 2A represents an exemplary experiment of the instant disclosure illustrating stability of Tat-PYC-Smad7 in the presence or absence of glycerol and separated by gel chromatography in accordance with certain embodiments disclosed herein.

FIG. 2B represents exemplary experiments of the instant disclosure illustrating non-reducing SDS-PAGE gel images of Tat-PYC-Smad7 formulated in various gelling agents of certain embodiments disclosed herein.

FIG. 2C represents exemplary experiments of the instant disclosure illustrating non-reducing SDS-PAGE gel images of Tat-PYC-Smad7 formulated in a variety of solutions and conditions in accordance with certain embodiments disclosed herein.

FIGS. 3A-3C represent exemplary experiments of the instant disclosure, illustrating characterization by size-exclusion chromatography (SEC) of representative formulations containing a Smad7 fragment protein and cellulose-based gelling agents in accordance with certain embodiments disclosed herein.

FIGS. 4A-4D represent exemplary experiments of the instant disclosure, illustrating gross scores and appearance of Tat-PYC-Smad7 treatments in dog oral mucositis with 4A-4B illustrating various conditions, 4C represents a graph of experimental conditions in the absence and presence of a SMAD7 construct; 4D, images of treated and untreated animal models in accordance with certain embodiments disclosed herein.

FIGS. 5A and 5B represent exemplary experiments of the instant disclosure, illustrating histology images of Tat-PYC-Smad7 treated mucositis in an animal model 5B compared to untreated animals 5A in accordance with certain embodiments disclosed herein.

FIGS. 6A-6C represent exemplary experiments of the instant disclosure, illustrating images of Tat-PYC-Smad7 targeting local tissue in the presence and absence of gelling agents (6A); in graphical representation of various conditions of an animal model (6B) and under dilution conditions to titrate the absorbance (6C) in accordance with certain embodiments of the present disclosure.

FIGS. 7A-7F represent an exemplary experiment of the instant disclosure, illustrating pharmacodynamics markers of Tat-PYC-Smad7-treated animal oral mucositis. 7A illustrates histological cross sections of experimental and control (vehicle) conditions; 7B represents a plot of the presence of the experimental construct in skin; 7C illustrates a plot of a vehicle control sample; 7D illustrates additional histology cross sections under various conditions and 7E and 7F represent graphical illustrations of the control versus experimental sample in accordance certain embodiments disclosed herein.

FIGS. 8A-8H represent exemplary experiments of the instant disclosure, illustrating that administration of Tat-PYC-Smad7 reduced of inflammatory cytokines as illustrated in 8A-8D released from oral mucositis lesions while 8E-8H illustrate graphical representations of experimental versus vehicle construct delivery on pro-inflammatory cytokine markers in accordance with certain embodiments disclosed herein.

FIGS. 9A-9D represent exemplary experiments of the instant disclosure, illustrating that administration of Tat-PYC-Smad7-HA in an exemplary gel and effect on alleviating imiquimod-induced skin inflammation in a mouse model where 9A represents images and corresponding histological images in titrated experimental and vehicle samples; 9B illustrates PASI scores of the various conditions in 9A and 9C illustrates two different parameters measured and plotted in a bar plot format to illustrate effects of experimental versus vehicle samples; and 9D are images of representative immunofluorescent stained samples of vehicle versus experimental conditions in accordance with certain embodiments disclosed herein.

FIG. 10 represents exemplary experiments of the instant disclosure, illustrating the stability of gel formulated, topically applied, exemplary constructs of Tat-PYC-Smad7-HA delivered to mouse wounds to illustrate the experimental effects of the construct versus gelling agent alone in accordance with certain embodiments disclosed herein.

FIG. 11 represents exemplary experiments of the instant disclosure, illustrating the penetration of gel formulated, topically applied, Tat-PYC-Smad7-HA in newly healed pig skin in accordance with certain embodiments disclosed herein.

FIGS. 12A-12B represent exemplary experiments of the instant disclosure, illustrating administration of Tat-PYC-Smad7-HA in gel and gel alone to mouse oral mucosa and skin at 2 hours (12A) and 24 hours (12B) in accordance with certain embodiments disclosed herein.

FIGS. 13A-13C represent exemplary experiments of the instant disclosure, illustrating that administration of Tat-PYC-Smad7-HA in gel alleviated radiodermatitis in mice where 13A illustrates the plot over time and a score after radiation; 13B includes photographs over time in control and treated sides of the mouse model and 13C illustrates the gel alone and in the presence of a construct in accordance with certain embodiments disclosed herein.

FIGS. 14A-14K represent exemplary experiments of the instant disclosure, illustrating presence of markers of control versus treated (Tat-PYC-Smad7 treatment) in mouse radiodermatitis (14A-14F) and graphical representations of the presence or absence of a marker in control versus experimental animals (14G-14K) in accordance with certain embodiments disclosed herein.

FIGS. 15A-15E represent exemplary experiments of the instant disclosure, illustrating better re-epithelialization of control versus treated (Tat-PYC-Smad7-HA treated) pig wounds where 15A and 15B represent histology sections and staining of various indicators and 15C-15E illustrate a graphical image of control versus experimental conditions and various physiological features of treatment assayed herein, in accordance with certain embodiments disclosed herein.

FIGS. 16A-16D represent exemplary experiments of the instant disclosure, illustrating control versus experimental (Tat-PYC-Smad7) alleviated skin inflammation induced by an experimental agent (IMQ) where 16A-16B represent exemplary photos of the conditions and 16C-16D illustrate stained images of the various conditions in accordance with certain embodiments disclosed herein.

FIGS. 17A-17D represent exemplary experiments of the instant disclosure, illustrating control versus experimental constructs (Tat-PY-Smad7 (203-217aa)) alleviated skin inflammation in an experimental setting in an animal model where each of 17A-17D represents photographic images and corresponding stained sections of these conditions where different gelling agents were also tested for effect on delivery in accordance with certain embodiments disclosed herein.

FIGS. 18A-18B represent exemplary experiments of the instant disclosure, illustrating control versus experimental (Tat-PYC-Smad7) alleviated skin inflammation induced by a therapeutic treatment in a model for contact dermatitis where 18A includes both photos and stained sections and 18B is a bar graph illustrating a physiological side effect or indicator of this condition in control versus treated animals in accordance with certain embodiments disclosed herein.

FIGS. 19A-19B represent exemplary experiments of the instant disclosure, illustrating control versus experimental (Tat-PYC-Smad7) alleviated atopic dermatitis in a tape-stripping mouse model where 19A includes both photos and stained sections and 19B is a bar graph illustrating a physiological side effect or indicator of this condition in control versus treated animals in accordance with certain embodiments disclosed herein.

FIGS. 20A-20B represent exemplary experiments of the instant disclosure, illustrating administration of Tat-PYC-Smad7-HA in gel and gel alone to wounds in diabetic pig skin (20A) where 20B illustrates representative stained sections of these conditions where different gelling agents were also tested for effect on delivery in accordance with certain embodiments disclosed herein.

FIG. 21 represents an exemplary experiment of the instant disclosure, illustrating a schematic of location of administration of Tat-PYC-Smad7-HA in gel and gel alone to wounds in healthy pig skin in accordance with certain embodiments disclosed herein.

FIGS. 22A-22E represent exemplary experiments of the instant disclosure, illustrating administration of Tat-PYC-Smad7-HA in gel and gel alone to wounds in heathy pig skin where 22A and 22B represent histology sections and staining of various indicators and 22C-22E illustrate a graphical image of control versus experimental conditions and various physiological features of treatment assayed herein, in accordance with certain embodiments disclosed herein.

FIGS. 23A-23D represent exemplary experiments of the instant disclosure, illustrating administration of Tat-PYC-Smad7-HA in gel and gel alone to wounds in heathy pig skin where 23A and 23C illustrate presence of markers of control versus treated (Tat-PYC-Smad7 treatment) in representative represent histology sections and graphical representations of the presence or absence of a marker in control versus experimental animals (23B and 23D) in accordance with certain embodiments disclosed herein.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods, or components have not been included in the description.

In some embodiments, formulations disclosed herein can include a recombinant Smad7, a fragment thereof, or fusion polypeptide thereof in solution or in combination with a gelling agent as an improved formulation for storage and delivery of these constructs. In certain embodiments, these formulations are used for improved delivery to a subject to treat a health condition.

In certain embodiments, the Smad7 polypeptide is a human or other mammalian derived polypeptide or recombinant molecule thereof. “Mothers against decapentaplegic homolog 7” or “Smad7” is a protein encoded by the SMAD7 gene. Smad7 is a member of the SMAD family which belong to the TGFβ (transforming growth factor beta) superfamily of ligands. Smad7 has several biological functions including, but not limited to, inhibiting TGFβ, activin, and/or bone morphogenetic protein (BMP) signaling to reduce or prevent association, phosphorylation and activation of downstream signaling targets, for example, Smad2.

In some embodiments, formulations disclosed herein for administering and/or delivering Smad7 polypeptides can include topical formulations of use to treat dermal and/or oral complications in a subject. In accordance with these embodiments, dermal and/or oral complications can be due to other underlying health issues or from treatments of underlying health issues including, but not limited to, side effects of therapeutic treatments. In certain embodiments, the dermal and/or oral complication can be a result of chemotherapy and/or radiation therapy administered to the subject as a side effect of these therapies. In some embodiments, topical formulations disclosed herein can include a recombinant human Smad7 polypeptide, a fragment thereof (e.g. a carboxyterminal or amino-terminal fragment or other fragment of about 5 consecutive amino acids or more of Smad7), or fusion polypeptide or fragment thereof and at least one gelling agent. In certain embodiments, the at least one gelling agent preserves or improves delivery of the recombinant human Smad7 polypeptide, the fragment thereof, or the fusion polypeptide or the fragment thereof by reducing degradation and/or facilitating delivery to a dermal surface or oral surface or maintaining consistency of the formulation (improved uniform distribution) for improved delivery of the active agent to the subject. In other embodiments, the formulation provides for improved half-life of the constructs and prolonged exposure to a targeted region of the subject for improved efficacy.

Some embodiments of the present disclosure provide compositions that can include at least one gelling agent and an isolated Smad7, recombinant Smad7, a fragment thereof, or fusion polypeptide thereof. In certain embodiments, the Smad7 is a human or other mammalian Smad7 or fragment thereof. In some embodiments, compositions or formulations disclosed herein can further include one or more protein stabilizers, one or more surfactants, one or more preservatives, one or more antimicrobial agents, one or more salts or any combination thereof. In some embodiments, formulations disclosed herein include one or more salt having a concentration of about 100 mM to about 2.5 M; or about 100 mM to about 1.75 M; or about 100 to about 1.5 M; about 100 mM to about 1.0 M; or about 100 mM to about 750 mM; or about 100 to about 500 mM; about 100 mM to about 400 mM; or about 100 mM to about 300 mM; or about 100 to about 200 mM; about 100 mM to about 175 mM; or about 100 to about 150 mM. In yet other embodiments, formulations disclosed herein can be adjusted or have a pH of about 6.0 to about 9.0 or about 7.0 to about 9.0. In some embodiments, formulations for delivery to a subject disclosed herein have a pH of about 7.0 to about 9.0 and a salt concentration of about 150 mM.

In some embodiments, the at least one gelling agent can have an average molecular weight (MW) of about 25,000 to about 1,500,000 Daltons. In other embodiments, the at least one gelling agent can be one or more celluloses or derivatives thereof. In accordance with these embodiments, the at least one gelling agent can include at least one cellulose agent or derivative thereof. In accordance with these embodiments, the least one cellulose agent or derivative thereof includes, but are not limited to, benzylcellulose (BC), ethylcellulose (EC), methylcellulose (MC), hydroxypropylmethylcellulose (HPMC), ethylhydroxyethylcellulose (EHEC), carboxymethylcellulose (CMC), hydroxypropylcellulose (HPC), hydroxyethylcellulose (HEC), hydroxyethylmethylcellulose (HEMC), sodium carboxymethylcellulose (CMCNa), cellulose acetate (CA), cellulose nitrate, cellulose sulphate, cellulose acetate phthalate (CAP), hydroxybutylcellulose, hydroxybutylmethylcellulose, carboxypropyl methylcellulose, hydroxypropyl methylcellulose (HPMC), cellulose acetate butyrate (CAB), microcrystalline cellulose (MCC), hydroxypropylmethylcellulose phthalate (HPMCP), cellulose acetate trimelitate (CAT), hydroxypropylmethylcellulose acetate succinate, or a combination thereof. In certain embodiments, the at least one cellulose or derivative thereof can include, but is not limited to, HPC, HEC, HPMC or any combination thereof. In other embodiments, the at least one cellulose or derivative thereof can include, but is not limited to, HEC, HPMC, or a combination thereof. In some embodiments, the at least one cellulose or derivative thereof in a composition herein can further include one or more protein stabilizers, one or more surfactants, one or more preservatives, one or more antimicrobial agents, one or more salts or any combination thereof in the presence of a SMAD7 polypeptide, fragment thereof or fusion polypeptide thereof.

Some embodiments of the present disclosure include compositions and formulations comprising at least one gelling agent including a cellulose agent or derivative thereof and at least one of an isolated Smad7, recombinant Smad7, a fragment thereof, or fusion polypeptide thereof. In accordance with these embodiments, the at least one cellulose or derivative thereof can include, but is not limited to, HPC, HEC, HPMC or any combination thereof. In other embodiments, the at least one cellulose or derivative thereof can include, but is not limited to, HEC, HPMC, or a combination thereof. In other embodiments, these formulations and compositions further include at least one salt and a pH of about 7.0 to about 9.0. In accordance with these embodiments, pH levels can be maintained to minimize denaturation of the constructs while salts and salt concentrations can be provided to reduce aggregation of the constructs in a solution or formulation disclosed herein. In other embodiments, the compositions or formulations disclosed herein can further include at least one surfactant agent (e.g., Tween, Tween 20, 40, 80 etc.), In yet other embodiments, formulations or compositions disclosed herein can further include glycerol or equivalent agent thereof.

In some embodiments, compositions or formulations disclosed herein can further include one or more protein stabilizers for enhancing protein stability and reducing protein degradation. In accordance with these embodiments, one or more protein stabilizers can include, but are not limited to, succinic anhydride, albumin, sialic acid, creatinine, glycine, histidine or other amino acid capable of stabilizing proteins, niacinamide, sodium acetyltryptophonate, zinc oxide, sucrose, glucose, lactose, trehalose, sorbitol, mannitol or other saccharide or disaccharide, glycerol, polyethylene glycols, sodium caprylate, sodium saccharin, or other suitable protein stabilizer or any combination thereof.

In some embodiments, compositions or formulations disclosed herein can further include one or more surfactants. In accordance with these embodiments, one or more surfactants of use in compositions disclosed herein can include, but are not limited to, sodium lauryl sulfate, sodium decussate, Tween-20, Tween-60, Tween-80; triacetin, vitamin E TPGS, a phospholipid, a lecithin, a phosphatidyl choline, a phosphatidylethanolamine, a phosphatidylglycerol, sorbitan monooleate, polyoxyethylene sorbitan monooleate, a polysorbate, a polaxomer, bile salt, glyceryl monostearate, a copolymer of ethylene oxide and propylene oxide, polyoxyethylene hydrogenated castor oil, polyoxyethylene alkylether, octoxynol 10, octoxynol 40, or any combination thereof.

In some embodiments, compositions disclosed herein can further include one or more preservatives including, but not limited to, benzalkonium chloride, chlorobutanol, thimerosol, chloroxylenol, chlorhexidine, phenoxyethanol, benzyl alcohol, phenethyl alcohol, polyquaterniaum-1, diazolidinyl urea, iodopropynyl butylcarbamate, chloromethylisotiazolinone, methylisothiazolinone, vitamin E, a vitamin E derivative, vitamin E acetate, vitamin C, butylated hydroxytoluene, butylparaben, ethylparaben, methylparaben, propylparaben, isobutylparaben, phenoxyethanol, ethylparaben, propylparaben, utylparaben, or any combination thereof. In some embodiments, compositions herein can be free of preservatives.

In certain embodiments, an isolated Smad7, a recombinant Smad7, a fragment thereof, or fusion polypeptide thereof of any Smad7 molecule is at least about 75% or more, or about 85% or more, or about 90% or more, or about 95% or more, or about 99%, or up to 100% identical to an amino acid sequence represented by SEQ ID NO: 1. In some embodiments, a recombinant human Smad7, a fragment thereof, or fusion polypeptide thereof for use herein can be encoded from a polynucleotide having at least about 75% or more, or about 85% or more, or about 90% or more, or about 95% or more, or about 99%, or up to 100% sequence homology to a nucleic acid sequence represented by SEQ ID NO: 2. In some embodiments, a recombinant human Smad7, a fragment thereof, or fusion polypeptide thereof for use herein has at least about 75% or more, or about 85% or more, or about 90% or more, or about 95% or more, or about 99%, or up to 100% sequence homology to an amino acid sequence represented by SEQ ID NO: 3. In some embodiments, a recombinant human Smad7, a fragment thereof, or fusion polypeptide thereof for use herein has at least about 75% or more, or about 85% or more, or about 90% or more, or about 95% or more, or about 99%, or up to 100% sequence homology to an amino acid sequence represented by SEQ ID NO: 4. In some embodiments, a recombinant human Smad7, a fragment thereof, or fusion polypeptide thereof for use herein has at least about 75% or more, or about 85% or more, or about 90% or more, or about 95% or more, or about 99%, or up to 100% sequence homology to an amino acid sequence represented by an amino acid sequence represented by SEQ ID NO: 5. In certain embodiments, formulations and compositions disclosed herein can include two or more SMAD7 constructs or fragments thereof in combination with a gelling agent or additional agents disclosed herein.

Human SMAD7 (UniProtKB/Swiss-Prot: O15105.1)
SEQ ID NO: 1
MFRTKRSALVRRLWRSRAPGGEDEEEGAGGGGGGGELRGEGATDSRAHGA
GGGGPGRAGCCLGKAVRGAKGHHHPHPPAAGAGAAGGAEADLKALTHSVL
KKLKERQLELLLQAVESRGGTRTACLLLPGRLDCRLGPGAPAGAQPAQPP
SSYSLPLLLCKVFRWPDLRHSSEVKRLCCCESYGKINPELVCCNPHHLSR
LCELESPPPPYSRYPMDFLKPTADCPDAVPSSAETGGTNYLAPGGLSDSQ
LLLEPGDRSHWCVVAYWEEKTRVGRLYCVQEPSLDIFYDLPQGNGFCLGQ
LNSDNKSQLVQKVRSKIGCGIQLTREVDGVWVYNRSSYPIFIKSATLDNP
DSRTLLVHKVFPGFSIKAFDYEKAYSLQRPNDHEFMQQPWTGFTVQISFV
KGWGQCYTRQFISSCPCWLEVIFNSR
Human SMAD7 (NCBI Reference Sequence: NM_005904.4)
SEQ ID NO: 2
ATTGCCTGCTTCTCCCCACCCCCAAATTAAGTTGCTTAGCAAGGGGGAAA
GAGGCTTTTTCCTTCTTCCAACAGCCCAGCCGAACGCCTTTCGTTTTTTG
CCCCCGCGGACCTTCCATGTAGGAAGCCGAGGCTGGCGAGCCCGACATTC
GGGAGCCACTGCGGGGGGGCCTCTTTTTGGGGAGGCGCCGACGGGGGCAG
GCTCGGCCGTCCCCAGGGAAGCGGCGGCCGGGTTCCTCCGGGGCGCGCCG
GGGCCGGAGAGCCGCGCAGGGCGCGGGCCGCGCGGGGTGGGGCAGCCGGA
GCGCAGGCCCCCGATCCCCGGCGGGCGCCCCCGGGCCCCCGCGCGCGCCC
CGGCCTCCGGGAGACTGGCGCATGCCACGGAGCGCCCCTCGGGCCGCCGC
CGCTCCTGCCCGGGCCCCTGCTGCTGCTGCTGTCGCCTGCGCCTGCTGCC
CCAACTCGGCGCCCGACTTCTTCATGGTGTGCGGAGGTCATGTTCGCTCC
TTAGCAGGCAAACGACTTTTCTCCTCGCCTCCTCGCCCCGCATGTTCAGG
ACCAAACGATCTGCGCTCGTCCGGCGTCTCTGGAGGAGCCGTGCGCCCGG
CGGCGAGGACGAGGAGGAGGGCGCAGGGGGAGGTGGAGGAGGAGGCGAGC
TGCGGGGAGAAGGGGCGACGGACAGCCGAGCGCATGGGGCCGGTGGCGGC
GGCCCGGGCAGGGCTGGATGCTGCCTGGGCAAGGCGGTGCGAGGTGCCAA
AGGTCACCACCATCCCCACCGCCAGCCGCGGGCGCCGGCGCGGCCGGGGG
CGCCGAGGCGGATCTGAAGGCGCTCACGCACTCGGTGCTCAAGAAACTGA
AGGAGCGGCAGCTGGAGCTGCTGCTCCAGGCCGTGGAGTCCCGCGGCGGG
ACGCGCACCGCGTGCCTCCTGCTGCCCGGCCGCCTGGACTGCAGGCTGGG
CCCGGGGGCGCCCGCCGGCGCGCAGCCTGCGCAGCCGCCCTCGTCCTACT
CGCTCCCCCTCCTGCTGTGCAAAGTGTTCAGGTGGCCGGATCTCAGGCAT
TCCTCGGAAGTCAAGAGGCTGTGTTGCTGTGAATCTTACGGGAAGATCAA
CCCCGAGCTGGTGTGCTGCAACCCCCATCACCTTAGCCGACTCTGCGAAC
TAGAGTCTCCCCCCCCTCCTTACTCCAGATACCCGATGGATTTTCTCAAA
CCAACTGCAGACTGTCCAGATGCTGTGCCTTCCTCCGCTGAAACAGGGGG
AACGAATTATCTGGCCCCTGGGGGGCTTTCAGATTCCCAACTTCTTCTGG
AGCCTGGGGATCGGTCACACTGGTGCGTGGTGGCATACTGGGAGGAGAAG
ACGAGAGTGGGGAGGCTCTACTGTGTCCAGGAGCCCTCTCTGGATATCTT
CTATGATCTACCTCAGGGGAATGGCTTTTGCCTCGGACAGCTCAATTCGG
ACAACAAGAGTCAGCTGGTGCAGAAGGTGCGGAGCAAAATCGGCTGCGGC
ATCCAGCTGACGCGGGAGGTGGATGGTGTGTGGGTGTACAACCGCAGCAG
TTACCCCATCTTCATCAAGTCCGCCACACTGGACAACCCGGACTCCAGGA
CGCTGTTGGTACACAAGGTGTTCCCCGGTTTCTCCATCAAGGCTTTCGAC
TACGAGAAGGCGTACAGCCTGCAGCGGCCCAATGACCACGAGTTTATGCA
GCAGCCGTGGACGGGCTTTACCGTGCAGATCAGCTTTGTGAAGGGCTGGG
GCCAGTGCTACACCCGCCAGTTCATCAGCAGCTGCCCGTGCTGGCTAGAG
GTCATCTTCAACAGCCGGTAGCCGCGTGCGGAGGGGACAGAGCGTGAGCT
GAGCAGGCCACACTTCAAACTACTTTGCTGCTAATATTTTCCTCCTGAGT
GCTTGCTTTTCATGCAAACTCTTTGGTCGTTTTTTTTTTGTTTGTTGGTT
GGTTTTCTTCTTCTCGTCCTCGTTTGTGTTCTGTTTTGTTTCGCTCTTTG
AGAAATAGCTTATGAAAAGAATTGTTGGGGGTTTTTTTGGAAGAAGGGGC
AGGTATGATCGGCAGGACACCCTGATAGGAAGAGGGGAAGCAGAAATCCA
AGCACCACCAAACACAGTGTATGAAGGGGGGCGGTCATCATTTCACTTGT
CAGGAGTGTGTGTGAGTGTGAGTGTGCGGCTGTGTGTGCACGCGTGTGCA
GGAGCGGCAGATGGGGAGACAACGTGCTCTTTGTTTTGTGTCTCTTATGG
ATGTCCCCAGCAGAGAGGTTTGCAGTCCCAAGCGGTGTCTCTCCTGCCCC
TTGGACACGCTCAGTGGGGCAGAGGCAGTACCTGGGCAAGCTGGCGGCTG
GGGTCCCAGCAGCTGCCAGGAGCACGGCTCTGTCCCCAGCCTGGGAAAGC
CCCTGCCCCTCCTCTCCCTCATCAAGGACACGGGCCTGTCCACAGGCTTC
TGAGCAGCGAGCCTGCTAGTGGCCGAACCAGAACCAATTATTTTCATCCT
TGTCTTATTCCCTTCCTGCCAGCCCCTGCCATTGTAGCGTCTTTCTTTTT
TGGCCATCTGCTCCTGGATCTCCCTGAGATGGGCTTCCCAAGGGCTGCCG
GGGCAGCCCCCTCACAGTATTGCTCACCCAGTGCCCTCTCCCCTCAGCCT
CTCCCCTGCCTGCCCTGGTGACATCAGGTTTTTCCCGGACTTAGAAAACC
AGCTCAGCACTGCCTGCTCCCATCCTGTGTGTTAAGCTCTGCTATTAGGC
CAGCAAGCGGGGATGTCCCTGGGAGGGACATGCTTAGCAGTCCCCTTCCC
TCCAAGAAGGATTTGGTCCGTCATAACCCAAGGTACCATCCTAGGCTGAC
ACCTAACTCTTCTTTCATTTCTTCTACAACTCATACACTCGTATGATACT
TCGACACTGTTCTTAGCTCAATGAGCATGTTTAGACTTTAACATAAGCTA
TTTTTCTAACTACAAAGGTTTAAATGAACAAGAGAAGCATTCTCATTGGA
AATTTAGCATTGTAGTGCTTTGAGAGAGAAAGGACTCCTGAAAAAAAACC
TGAGATTTATTAAAGAAAAAAATGTATTTTATGTTATATATAAATATATT
ATTACTTGTAAATATAAAGACGTTTTATAAGCATCATTATTTATGTATTG
TGCAATGTGTATAAACAAGAAAAATAAAGAAAAGATGCACTTTGCTTTAA
TATAAATGCAAATAACAAATGCCAAATTAAAAAAGATAAACACAAGATTG
GTGTTTTTTTCTATGGGTGTTATCACCTAGCTGAATGTTTTTCTAAAGGA
GTTTATGTTCCATTAAACGATTTTTAAAATGTACACTTGA
Human SMAD7 (aa 203-426)
SEQ ID NO: 3
ELESPPPPYSRYPMDFLKPTADCPDAVPSSAETGGTNYLAPGGLSDSQLL
LEPGDRSHWCVVAYWEEKTRVGRLYCVQEPSLDIFYDLPQGNGFCLGQLN
SDNKSQLVQKVRSKIGCGIQLTREVDGVWVYNRSSYPIFIKSATLDNPDS
RTLLVHKVFPGFSIKAFDYEKAYSLQRPNDHEFMQQPWTGFTVQISFVKG
WGQCYTRQFISSCPCWLEVIFNSR
Human SMAD7 (aa 259-426)
SEQ ID NO: 4
TRVGRLYCVQEPSLDIFYDLPQGNGFCLGQLNSDNKSQLVQKVRSKIGCG
IQLTREVDGVWVYNRSSYPIFIKSATLDNPDSRTLLVHKVFPGFSIKAFD
YEKAYSLQRPNDHEFMQQPWTGFTVQISFVKGWGQCYTRQFISSCPCWLE
VIFNSR
Human SMAD7 (aa 203-217)
SEQ ID NO: 5
ELESPPPPYSRYPMD

In certain embodiments, an isolated Smad7, a recombinant Smad7, a fragment thereof, or fusion polypeptide thereof of any Smad7 molecule can further include at least one protein transduction domain (PTD). PTDs, also referred to as “cell penetrating peptides” (CPPs), can be rapidly internalized into a cell across its biological membrane. PTDs generally are relatively short amino acid sequences that can be linked to a therapeutic protein of interest (e.g., an isolated Smad7, a recombinant Smad7, a fragment thereof, or fusion polypeptide thereof of any Smad7 molecule), allowing transport of the therapeutic protein across a biological membrane, such as a cell membrane, an organelle membrane, and/or a nuclear membrane. In some embodiments, a recombinant human Smad7, a fragment thereof, or fusion polypeptide thereof for use herein can further comprise at least one PTD or a variant thereof wherein the PTD can be Antennapedia (Antp), herpes simplex virus type 1 (HSV-1) protein VP22, secretory leukocyte protease inhibitor (SLPI), HIV-derived cationic TAT, human calcitonin (hCT), transportan, polyarginine sequences (Arg8), azurin p28, Pep-1 (SV40 T antigen NLS), other PTDs and the like. According to some embodiments herein, a recombinant human Smad7, a fragment thereof, or fusion polypeptide thereof disclosed herein can further include at least one PTD or a variant thereof as provided in Table 1 or any combination thereof or other known PTD or polypeptide capable of functioning as a PTD.

TABLE 1

| | | SEQ |
|---|---|---|
| | | ID |
| PTD | Amino Acid Sequence | NO: |
| TAT (47-57) | YGRKKRRQRRR | 6 |
| TAT (48-60) | GRKKRRQRRRPPQ | 7 |
| TAT (49-57) | RKKRRQRRR | 8 |
| TAT (48-57) | GRKKRRQRRR | 9 |
| Antp-43-58 | RQIKIWFQNRRMKWKK | 10 |
| hCT (9-32) | LGTYTQDFNKFHTFPQTAIGVGAP | 11 |
| Pep-1 | KETWWETWWTEWSQPKKKRKV | 12 |
| MAP | KLALKLALKALKAALKLA | 13 |
| Transportan | GWTLNSAGYLLGKINLKALAALAKKIL | 14 |
| pVEC | LLIILRRRIRKQAHAHSK | 15 |
| Azurin p28 | LSTAADMQGVVTDGMASGLDKDYLKPDD | 16 |
| (50-77) | | |
| VP22 | NAATATRGRSAASRPTQRPRAPARSASR | 17 |
| | PRRPVQ | |
| ARF (1-22) | MVRRFLVTLRIRRACGPPRVRV | 18 |
| VT5 | DPKGDPKGVTVTVTVTVTGKGDPKPD | 19 |
| C105Y | CSIPPEVKFNKPFVYLI | 20 |
| FGF | PIEVCMYREP | 21 |
| Pep-7 | SDLWEMMMVSLACQY | 22 |
| PFV | PFVYLI | 23 |
| Vectocell | CVKRGLKLRHVRPRVTRMDV | 24 |

In other embodiments, an isolated Smad7, a recombinant Smad7, a synthetically generated Smad7, a fragment thereof, or fusion polypeptide thereof of any Smad7 molecule can further include at least one PTD or a variant or mutant thereof having at least about 75% or more, or about 85% or more, or about 90% or more, or about 95% or more, or about 99%, or up to 100% sequence homology to an amino acid sequence represented by any one of SEQ ID NOs: 6-25. In some embodiments, an isolated Smad7, a recombinant Smad7, a synthetically generated Smad7, a fragment thereof, or fusion polypeptide thereof of any Smad7 molecule can further include at least one TAT PTD or a variant or mutant thereof. In some embodiments, the PTD can be a PTD capable of transporting a polypeptide to the nucleus. In some embodiments, an isolated Smad7, a recombinant Smad7, a fragment thereof, or fusion polypeptide thereof of any Smad7 molecule disclosed herein can further include at least one TAT PTD or a variant thereof having at least about 75% or more, or about 85% or more, or about 90% or more, or about 95% or more, or about 99%, or up to 100% sequence homology to an amino acid sequence represented by SEQ ID NOs: 6-9. In some embodiments, a recombinant human Smad7, a fragment thereof, or fusion polypeptide thereof disclosed herein can further include at least one TAT PTD that having at least about 75% or more, or about 85% or more, or about 90% or more, or about 95% or more, or about 99%, or up to 100% sequence homology to an amino acid sequence represented by SEQ ID NO: 9.

In some embodiments, at least one PTD or a variant thereof can be fused to the N-terminus, the C-terminus, or the N- and the C-terminus of an isolated Smad7, recombinant Smad7, a fragment thereof, or fusion polypeptide thereof as disclosed herein. One of skill in the art can appreciate that standard methods of molecular biology can be used to attach any PTD or variants thereof to an isolated Smad7, a recombinant human Smad7, a fragment thereof, or fusion polypeptide thereof of any Smad7 molecule as disclosed herein. In some embodiments, one or more PTD or variants thereof can be fused to an isolated Smad7, to a recombinant Smad7, a fragment thereof, or fusion polypeptide thereof as disclosed herein by covalent linkage. In accordance with these embodiments, one or more PTD or variants thereof can be fused to a to an isolated Smad7, a recombinant Smad7, a fragment thereof, or fusion polypeptide thereof as disclosed herein through a disulfide bond (e.g., by modifying PTD and peptide/protein with cysteine), an electrostatic or hydrophobic interaction, and/or through cross-linkers. In some embodiments, one or more PTD or variants thereof can be connected to a recombinant human Smad7, a fragment thereof, or fusion polypeptide thereof as disclosed herein by a linker between the PTD and the Smad7 protein. In some embodiments, a linker between a PTD, a fusion polypeptide or variant thereof and an isolated Smad7, a recombinant Smad7, a fragment thereof, or fusion polypeptide thereof as disclosed herein can be about 1 to about 30 amino acids (e.g., about 1, 5, 10, 15, 20, 25, 30).

In some embodiments, an isolated Smad7, a recombinant Smad7, a fragment thereof, or fusion polypeptide thereof as disclosed herein can further include at least one epitope tag. As used herein, an “epitope tag” refers to a biological structure, usually a polypeptide that can be genetically engineered onto either the N- and/or C-terminus of a recombinant protein. Epitope tags herein can be detected by one or more antibodies and generally do not compromise the native structure or function of the protein. In some embodiments, epitope tags herein can be attached to an isolated Smad7, a recombinant human Smad7, a fragment thereof, or fusion polypeptide thereof of any Smad7 molecule as disclosed herein to allow for assessment and/or purification of the Smad7 protein using one or more immunoassays. Non-limiting immunoassays for use with any of the epitope tags described herein can include western blotting, immunoprecipitation, immunohistochemistry, immunofluorescence staining, immunocytochemistry, flow cytometry, and the like. In some embodiments, epitope tags suitable for use herein can include HA, HIS, FLAG, AU1, AU5, Myc, Glu-Glu, OLLAS, T7, V5, VSV-G, E-Tag, S-Tag, Avi, HSV, KT3, TK15, Strep-tag II, Beta-Galactosidase, Maltose Binding Protein (MBP), Calmodulin Binding Protein (CBP), Green Fluorescent Protein (GFP), Glutathione S Transferase (GST), mCherry, a Fc-Fusion Protein, Thioredoxin, any variant thereof, and in any combination thereof. In some embodiments, at least one epitope tag or a variant thereof can be placed at the N-terminus, at the C-terminus, or at the N- and the C-terminus of a recombinant human Smad7, a fragment thereof, or fusion polypeptide thereof as disclosed herein wherein the at least one epitope tag or a variant thereof can be HA, V5, GST, or any combination and/or variant thereof. In some embodiments, at least two epitope tags or a variants thereof can be placed at the N-terminus, at the C-terminus, or at the N- and the C-terminus of an isolated Smad7, a recombinant human Smad7, a fragment thereof, or fusion polypeptide thereof of any Smad7 molecule as disclosed herein wherein the at least two epitope tags or a variants thereof can be HA, V5, GST, or any combination and/or variant thereof.

In some embodiments, at least one epitope tag or a variant thereof can be placed at the N-terminus, at the C-terminus, or at the N- and the C-terminus of a recombinant human Smad7, a fragment thereof, or fusion polypeptide thereof disclosed herein. Any method known in the art can be used to attach an epitope tag or variants thereof to a Smad7 molecule contemplated herein. In some embodiments, at least two epitope tags or variants thereof can be fused to the N- and the C-terminus of an isolated Smad7, a recombinant Smad7, a fragment thereof, or fusion polypeptide thereof of any Smad7 molecule as disclosed herein. In some embodiments, at least one epitope tag or a variant thereof fused to the N-terminus, at the C-terminus, or at the N- and the C-terminus of a Smad7 molecule as disclosed herein can be removed from the Smad7 by cleavage at one or more cleavage sites if needed for further formulation and/or use in a composition disclosed herein.

In some embodiments, compositions disclosed herein can include an isolated Smad7, a recombinant human Smad7, a synthetically generated Smad7, a fragment thereof, or fusion polypeptide thereof of any Smad7 molecule at a concentration about 0.5 ng/ml to about 5 mg/ml, or about 0.5, 0.75, 1.0, 1.25, 1.5, 1.75, 2.0, 2.5, 3.0, 3.5, 4.0, 4.5, 5.0, 6.0, 7.0, 8.0, 9.0, 10, 15, 20, 25, 30, 35, 40, 45, 50, 55, 60, 65, 70, 75, 80, 85, 90, 95, 100, 110, 125, 150, 175, 200, 250, 300, 350, 400, 450, 500, 600, 700, 800, 900, 1000 ng/ml; or about 0.5, 0.75, 1.0, 1.25, 1.5, 1.75, 2.0, 2.5, 3.0, 3.5, 4.0, 4.5, 5.0, 6.0, 7.0, 8.0, 9.0, 10, 15, 20, 25, 30, 35, 40, 45, 50, 55, 60, 65, 70, 75, 80, 85, 90, 95, 100, 110, 125, 150, 175, 200, 250, 300, 350, 400, 450, 500, 600, 700, 800, 900, 1000, 1200, 1200, 1300, 1400, 1500, 1600, 1700, 1800, 1900, 2000, 1250, 1500, 2750, 3000, 3250, 3500, 3750, 4000, 4250, 4500, 4750, 5000 μg/ml or higher concentration.

In some embodiments, compositions or formulations disclosed herein can be formulated for topical, mucosal, or dermal administration. In certain embodiments, formulations suitable for topical administration can be a solution, an emulsion, a suspension, a drop, a cream, a lotion, a paste, an ointment, a gel, or any combination thereof. In accordance with these embodiments, formulations suitable for topical, mucosal, or dermal administration include a gelling agent and a therapeutic polypeptide disclosed herein (e.g., an isolated Smad7, a recombinant Smad7, a fragment thereof, or fusion polypeptide thereof of any Smad7 polypeptide). In some embodiments, a Smad7 fragment can include amino acids 203-258 of human Smad7 wherein amino acids 203-258 of human Smad7 can be referred to as a “PY-domain” herein. In some embodiments, a Smad7 fragment can include amino acids 259-426 of human Smad7 wherein amino acids 259-426 of human Smad7 can be referred to as a “C-domain” herein. In some embodiments, a Smad7 fragment can include amino acids 203-426 of human Smad7 wherein amino acids 203-426 of human Smad7 can be referred to as a “PYC-domain” herein. In some embodiments, a Smad7 fragment can include amino acids 203-426 of human Smad7 (i.e., a PYC domain) fused to a tat PTD. In accordance with these embodiments, a Smad7 construct of use in formulations disclosed herein can be a Tat-PYC-Smad7. In some embodiments, the construct can further include a gelling agent disclosed herein to facilitate delivery and for storage. In other embodiments, Tat-PYC-Smad7 can be formulated and then administered to a mucosal surface of a subject in need of such a treatment, before, during and/or after other treatments of a health condition.

As used herein, the term “gelling agent” can refer to a material that thickens and/or stabilizes solutions, emulsions, suspensions, creams, lotions, ointments, gels, or any combination of such compositions contemplated herein. Non-limiting examples of gelling agents may include, among others, carboxypolymethylene, Veegum, poloxamers, carrageenan, Irish moss, gums (such as gum karaya, gum arabic, gum tragacanth, xanthan gum, etc.), starch, alginate, polyvinylpyrrolidone, celluloses (such as hydroxyethyl propylcellulose, hydroxybutyl methyl cellulose, hydroxypropyl methyl cellulose, hydroxyethyl cellulose, carboxymethyl cellulose, carboxypropyl cellulose, and/or the like), carboxyvinyl polymers, hydroxyvinyl polymers, Carbopol, polymers, colloidal silica, and/or complex colloidal magnesium aluminum silicates, or any combination thereof. In some embodiments, compositions disclosed herein can include about 0.01% to about 25.0%. For example, a solution can be about 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 12%, 14%, 16%, 18%, 20%) weight per volume (w/v) of at least one gelling agent.

In other embodiments, compositions disclosed herein can include one or more celluloses or cellulose derivatives (also referred to as cellulosics) as gelling agents. In some embodiments, one or more celluloses or derivatives thereof for use as a gelling agent in the compositions disclosed herein can include, but are not limited to, a benzylcellulose (BC), ethylcellulose (EC), methylcellulose (MC), hydroxypropylmethylcellulose (HPMC), ethylhydroxyethylcellulose (EHEC), carboxymethylcellulose (CMC), hydroxypropylcellulose (HPC), hydroxyethylcellulose (HEC), hydroxyethylmethylcellulose (HEMC), sodium carboxymethylcellulose (CMCNa), cellulose acetate (CA), cellulose nitrate, cellulose sulphate, cellulose acetate phthalate (CAP), hydroxybutylcellulosc, hydroxybutylmethylcellulose, carboxypropyl methylcellulose, hydroxypropyl methylcellulose (HPMC), cellulose acetate butyrate (CAB), microcrystalline cellulose (MCC), hydroxypropylmethylcellulose phthalate (HPMCP), cellulose acetate trimelitate (CAT), hydroxypropylmethylcellulose acetate succinate, or other cellulose agent or any combination thereof. In some embodiments, one or more celluloses or derivatives thereof of use as gelling agents in compositions disclosed herein can be one or more of HEC, HPC, HPMC, or a combination thereof.

In some embodiments, one or more celluloses or derivatives thereof of use as a gelling agent in the compositions disclosed herein can have an average molecular weight (MW) between about 25,000 to about 1,500,000 Daltons. In other embodiments, one or more celluloses or derivatives thereof for use as gelling agent in the compositions disclosed herein can have a typical viscosity (mPas) between about 10 mPas to about 50,000 mPas (e.g., about 10, 100, 500, 1,000, 5,000, 10,000, 15,000, 20,000, 25,000, 30,000, 35,000, 40,000, 45,000, 50,000). In other embodiments, compositions disclosed herein can include about 0.01% to about 25.0% w/v or about 0.01%, 0.05%, 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 12%, 14%, 16%, 18%, 20% weight per volume (w/v) of one or more cellulose agents or derivatives thereof. In some embodiments, formulations can include, but are not limited to, HPC, HEC, HPMC, or a combination thereof. In certain embodiments, concentration of one or more celluloses or derivatives thereof of use as a gelling agent can be about 0.01% to about 25% (e.g., about 0.01%, 0.05%, 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10%, 12%, 14%, 16%, 18%, 20% weight per volume (w/v) HPC, HEC, HPMC, or a combination thereof.

In some embodiments, compositions disclosed herein can include one or more gelling agent wherein the total concentration of gelling agents can be sufficient to increase viscosity of the composition to permit the composition to remain in the applied location for an extended period or to facilitate absorption of the composition. Viscosity in a composition can be expressed in units of centipoise (cP). Viscosity can be measured using methods commonly known in the art. In some embodiments, a LVDV-II+CP Cone Plate Viscometer and/or a Cone Spindle CPE-40 can be used to calculate the viscosity of the compositions disclosed herein.

In some embodiments, compositions disclosed herein can include one or more gelling agents wherein the total concentration of gelling agents can be sufficient to provide an apparent viscosity from about 100 to about 100,000 cP. In some embodiments, an apparent viscosity of a composition disclosed herein can range from about 100 cP to about 50,000 cP, about 100 cP to about 1,000 cP, about 500 cP to about 1500 cP, about 1000 cP to about 3000 cP, about 2000 cP to about 8,000 cP, about 4,000 cP to about 50,000 cP, about 10,000 cP to about 500,000 cP, or about 15,000 cP to about 1,000,000 cP. In some embodiments, the viscosity ranges referred to herein can be measured at room temperature. In other embodiments, the viscosity ranges referred to herein can be measured at body temperature (e.g., at the average body temperature of a healthy human).

In some embodiments, compositions disclosed herein can include one or more cellulose derivatives and one or more bioadhesive polymers. Bioadhesive polymers are synthetic, semi synthetic, or natural macromolecules with capability of attaching to skin or mucosal surfaces. In some embodiments, compositions disclosed herein can include one or more cellulose derivatives mixed with one or more bioadhesive polymers to improve the adhesion characteristics of the composition such as adhesion time and adhesion force. Non-limiting examples of concurrent use of one or more cellulose derivatives and one or more bioadhesive polymers can include polyvinyl pyrrolidone (PVP), hydroxypropyl beta cyclodextrin, polycarbophil, carbopol(s), pectin, dextran and/or mannitol with HPMC, HEC and/or NaCMC.

In some embodiments, compositions disclosed herein can include additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some embodiments, compositions or formulations disclosed herein can include about 0.1% to about 99% or about 0.1%, 0.5%, 1%, 5%, 10%, 20%, 30%, 40%, 50%, 60%, 70%, 80%, 90%, 95%, or about 99% total amount of one or more agents by total weight of the composition. In some embodiments, compositions disclosed herein can include one or more of these agents to improve the performance, efficacy, safety, shelf-life and/or other property of an isolated Smad7, recombinant Smad7, a fragment thereof, or fusion polypeptide thereof disclosed herein. In accordance with these embodiments, additional agents or additives included in these compositions can be biocompatible, and in certain embodiments, they cannot be harsh, abrasive, or allergenic.

In some embodiments, compositions and formulations containing one or more Smad7 molecule or fusion polypeptide disclosed herein can include one or more acidifying agents. As used herein, “acidifying agents” refers to compounds used to provide an acidic medium or adjust pH. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some embodiments, any pharmaceutically acceptable organic or inorganic acid can be used herein. In some embodiments, compositions disclosed herein can include about 0.1% to about 99% (e.g., about 0.1%, 0.5%, 1%, 5%, 10%, 20%, 30%, 40%, 50%, 60%, 70%, 80%, 90%, 95%, 99%) total amount of one or more acidifying agents by total weight of the composition.

In some embodiments, compositions disclosed herein can include one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some embodiments, any pharmaceutically acceptable organic or inorganic base can be used in compositions herein. In some embodiments, compositions disclosed herein can include at most about 0.1% to about 99% (e.g., about 0.1%, 0.5%, 1%, 5%, 10%, 20%, 30%, 40%, 50%, 60%, 70%, 80%, 90%, 95%, 99%) of one or more alkalizing agents by total weight of the composition.

In some embodiments, compositions disclosed herein can include one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some embodiments, compositions disclosed herein can include at most about 0.1% to about 99% (e.g., about 0.1%, 0.5%, 1%, 5%, 10%, 20%, 30%, 40%, 50%, 60%, 70%, 80%, 90%, 95%, 99%) total amount of one or more antioxidants by total weight of the composition.

In some embodiments, compositions disclosed herein can include a buffer system. As used herein, a “buffer system” is a composition having one or more buffering agents wherein “buffering agents” are compounds used to maintain solution pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some embodiments, any pharmaceutically acceptable organic or inorganic buffer can be used. In some embodiments, compositions disclosed herein can include at most about 0.1% to about 99% (e.g., about 0.1%, 0.5%, 1%, 5%, 10%, 20%, 30%, 40%, 50%, 60%, 70%, 80%, 90%, 95%, 99%) total amount of one or more buffering agents by total weight of the composition.

In some embodiments, amounts of one or more buffering agents included in compositions herein can depend on the desired pH level of a composition. In some embodiments, compositions disclosed herein further including one or more buffering agents can maintain a pH of the composition at the desired level during storage of the composition. In some embodiments, compositions disclosed herein further including one or more buffering agents can maintain the pH of the composition after it is topically applied and exposed to the environment.

In some embodiments, compositions disclosed herein can have a pH of about 6.0 to about 9.5, about 6.5 to about 9.0, about 7 to about 9. In some embodiments, compositions disclosed herein can have a pH greater than about 8.5, greater than about 8, greater than about 8.5, greater than about 8, greater than about 7.5, greater than about 7, greater than about 6.5, greater than about 6. In some embodiments, compositions disclosed herein can have a pH greater between about 6 to about 9.5 (e.g., about 6.1 to 9.0). In some embodiments, compositions disclosed herein can have a pH of at least about pH 7.0. In other embodiments, compositions disclosed herein can have a pH of about pH 7.0 to about pH 9.0. In certain embodiments, compositions disclosed herein can have a pH of about pH 7.0-8.0.

In some embodiments, compositions described herein can be stable with respect to pH over a period of any of at least about 1 day, at least about 2 days, at least about 3 days, at least about 1 weck, at least about 2 weeks, at least about 3 weeks, at least about 4 weeks or more, at least about 1 month, at least about 2 months, at least about 3 months, at least about 4 months, at least about 5 months, at least about 6 months, at least about 12 months, at least about 24 months, at least about 36 months, or more. In some embodiments, compositions described herein can be stable with respect to pH over a period of at least about a week to about a month. In some embodiments, compositions described herein can be stable with respect to pH over a period of at least about a weck to about 4 years (e.g., about 1 week, 1 month, 6 months, 1 year, 1.5 years, 2 years, 2.5 years, 3 years, 3.5 years, 4 years) when stored at -80° C.

In some embodiments, compositions disclosed herein can include one or more preservatives. As used herein, “preservatives” refers to agents or combination of agents that inhibits, reduces or eliminates bacterial growth or maintains integrity of the agents in a composition or pharmaceutically acceptable composition. In some embodiments, compositions disclosed herein can include one or more preservative. Suitable preservatives for use in compositions and formulations disclosed herein include, but are not limited to, benzalkonium chloride, chlorobutanol, -, chloroxylenol, chlorhexidine, phenoxyethanol, benzyl alcohol, phenethyl alcohol; polyquaterniaum-1, diazolidinyl urea, iodopropynyl butylcarbamate, chloromethylisotiazolinone, methylisothiazolinone, vitamin E, vitamin E derivative, vitamin E acetate, vitamin C, butylated hydroxytoluene, butylparaben, ethylparaben, propylparaben, isobutylparaben, phenoxyethanol, ethylparaben, utylparaben, mixtures of the foregoing and the like. In some embodiments, a preservative can be, by way of example only, an antimicrobial agent, within the compositions and formulations presented herein. In some embodiments, in compositions and formulations disclosed herein can include a preservative such as by way of example only, methyl paraben. In some embodiments, compositions described herein can include a preservative at a concentration of about 0.01% to about 5.0% (e.g., about 0.01%, 0.05%, 0.1%, 0.5%, 1.0%, 1.5%, 2.0%, 2.5%, 3.0%, 3.5%, 4.0%, 4.5%, 5.0%). In some embodiments, compositions or formulations disclosed herein are free of preservatives to reduce exposure to preservatives or allergic reaction or other side effect due to presence of preservatives.

In some embodiments, compositions described herein can include one or more surfactants. As used herein, “surfactants” refer to compounds that lower the surface tension between two liquids, between a gas and a liquid, or between a liquid and a solid and or serve to protect agents from degradation and facilitate stability and delivery of a target agent. Surfactants may act, for example, as detergents, wetting agents, emulsifiers, foaming agents, and/or dispersants. In some embodiments, surfactants herein can be synthetic, natural, and/or semi-synthetic. In some embodiments, compositions disclosed herein can include anionic surfactants, cationic surfactants, zwitterionic surfactants, ampholytic surfactants, amphoteric surfactants, nonionic surfactants having a steroid skeleton, or a combination thereof. In some embodiments, compositions disclosed herein can include at least one surfactant wherein the surfactant can be sodium lauryl sulfate; sodium decussate; Tween-20, Tween-60; Tween-80; triacetin; vitamin E TPGS; phospholipids; lecithins; phosphatidyl cholines (c8-c18); phosphatidylethanolamines (c8-c18); phosphatidylglycerols (c8-c18); sorbitan monooleate; polyoxyethylene sorbitan monooleate; polysorbates; polaxomers; bile salts; glyceryl monostearate; copolymers of ethylene oxide and propylene oxide (e.g., Pluronic (BASF)); poloxamers and the like such as poloxamer 407 or 403; polyoxyethylene fatty acid glycerides and vegetable oils (e.g., polyoxyethylene (60) hydrogenated castor oil); and, polyoxyethylene alkylethers and alkylphenyl ethers (e.g., octoxynol 10, octoxynol 40). In some embodiments, compositions disclosed herein can include at most about 0.0001% to about 99% (e.g., about 0.0001%, 0.0005%, 0.001%, 0.005%, 0.01%, 0.05%, 0.1%, 0.5%, 1%, 5%, 10%, 20%, 30%, 40%, 50%, 60%, 70%, 80%, 90%, 95%, 99%) total amount of one or more surfactants by total weight of the composition. In certain embodiments, a surfactant in compositions disclosed herein include Tween. In accordance with these embodiments a surfactant including but not limited to Tween can be about 0.0001 to about 0.1% (w/v).

In some embodiments, compositions described herein can include one or more protein stabilizers. As used herein, a “protein stabilizer” refers to a compound used to stabilize a target polypeptide (e.g., an isolated Smad7, a recombinant Smad7, a fragment thereof, or fusion polypeptide thereof disclosed herein) against physical, chemical, thermal and/or biochemical processes that could otherwise reduce the activity of the target polypeptide. In some embodiments, protein stabilizers herein can be anti-oxidation agents, buffers, acids, preservatives and the like that are compatible with the environment of the targeted area (e.g., dermis, oral mucosa). Protein stabilizers include but are not limited to agents that may (1) improve the compatibility of excipients within a container, or a delivery system, including a syringe or a glass bottle, (2) improve the stability of a component of the composition such as a polypeptide, (3) improve formulation stability, or a combination thereof for manufacturing, storage and delivery. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, tween-20, tween-80, polyethylene glycols, sodium caprylate and sodium saccharin and others known to those of ordinary skill in the art. In some embodiments, compositions disclosed herein can include at most about 0.001% to about 99% (e.g., about 0.001%, 0.005%, 0.01%, 0.05%, 0.1%, 0.5%, 1%, 5%, 10%, 20%, 30%, 40%, 50%, 60%, 70%, 80%, 90%, 95%, 99%) total amount of one or more protein stabilizers by total weight of the composition.

In some embodiments, compositions described herein can be stable with respect to reducing degradation of a target polypeptide (e.g., an isolated, a recombinant Smad7, a fragment thereof, or fusion polypeptide thereof) for about 1 day, at least about 2 days, at least about 3 days, at least about 4 days, at least about 5 days, at least about 6 days, at least about 1 week, at least about 2 weeks, at least about 3 weeks, at least about 4 weeks, at least about 1 month, at least about 2 months, at least about 3 months, at least about 4 months, at least about 5 months, or at least about 6 months or more. In some embodiments, formulations can be stabilized for about a day to about 6 months or more when frozen, or stored at a temperature of about 4° C. to about 60° C. In some embodiments, compositions described herein can be stable with respect to lack of degradation of the therapeutic protein (e.g., an isolated Smad7, a recombinant Smad7, a fragment thereof, or fusion polypeptide thereof) for up to about 3 months when stored at a temperature of about 4° C. to about 37° C. (e.g., about 4° C., 10° C., 15° C., 20° C., 25° C., 30° ° C., or about 35° C.).

In some embodiments, compositions described herein can include one or more tonicity agents. As used herein, a “tonicity agent” refers to a compound that can be used to adjust the tonicity of the formulation herein. Suitable tonicity agents include, but are not limited to, glycerin, lactose, mannitol, dextrose, sodium chloride, sodium sulfate, sorbitol, trehalose, and others known to those or ordinary skill in the art. Osmolarity in a composition can be expressed in milliosmoles per liter (mOsm/L). Osmolarity may be measured using methods commonly known in the art. In some embodiments, a vapor pressure depression method can be used to calculate the osmolarity of the compositions disclosed herein.

In some embodiments, compositions disclosed herein can include one or more tonicity agents creating a composition having an osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some embodiments, compositions described herein can include one or more tonicity agents to result in a composition having an osmolarity ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a composition described herein has an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some embodiments, compositions disclosed herein can include at most about 0.1% to about 99% (e.g., about 0.1%, 0.5%, 1%, 5%, 10%, 20%, 30%, 40%, 50%, 60%, 70%, 80%, 90%, 95%, 99%) total amount of one or more toxicity modifiers by total weight of the composition.

In some embodiments, compositions herein can include one or more antifoaming agents. As used herein, an “antifoaming agent” refers to a compound that prevents or reduces the amount of foaming that might form on the surface of a composition. Suitable antifoaming agents include by way of example and without limitation, dimethicone, simethicone, octoxynol and others known to those of ordinary skill in the art. In some embodiments, compositions disclosed herein can include at most about 0.1% to about 99% (e.g., about 0.1%, 0.5%, 1%, 5%, 10%, 20%, 30%, 40%, 50%, 60%, 70%, 80%, 90%, 95%, 99%) total amount of one or more antifoaming agents by total weight of the composition.

In some embodiments, compositions herein can include one or more sweetening agents. Non-limiting examples of sweetening agents suitable for use herein can include aspartame, dextrose, glycerin, malitol, mannitol, saccharin sodium, sorbitol, sucrose, xylitol, and the like. Such sweetening agents can be added to increase likelihood of patient compliance. In some embodiments, compositions disclosed herein can include at most about 0.1% to about 99% (e.g., about 0.1%, 0.5%, 1%, 5%, 10%, 20%, 30%, 40%, 50%, 60%, 70%, 80%, 90%, 95%, 99%) total amount of one or more sweetening agents by total weight of the composition.

In some embodiments, compositions herein can include one or more enhancing agents. As used herein, an “enhancing agent” refers to a compound that aids in an increase or prolongation of either the potency or duration of a desired effect of a therapeutic protein (e.g., a recombinant human Smad7, a fragment thereof, or fusion polypeptide thereof disclosed herein) or a diminution of any adverse symptomatology that is consequent upon the administration of the therapeutic protein. In some embodiments, compositions disclosed herein can include at most about 0.1% to about 99% (e.g., about 0.1%, 0.5%, 1%, 5%, 10%, 20%, 30%, 40%, 50%, 60%, 70%, 80%, 90%, 95%, 99%) total amount of one or more enhancing agents by total weight of the composition. In some embodiments, an enhancing agent(s) for use herein can include, but is not limited to, dimethylsulfoxide (DMSO) or a combination of pluronic lecithin organizer (PLO), DMSO, alcohols, such as short chain alcohols, long chain alcohols, or polyalcohols, amines and amides, such as urea, amino acids or their esters, amides, AZONE (n-dodecyl-caprolactam), derivatives of AZONE, pyrrolidones, or derivatives of pyrrolidones; terpenes and derivatives of terpenes; fatty acids and their esters; macrocyclic compounds; tensides; or sulfoxides other than dimethylsulfoxide, such as, decylmethylsulfoxide; liposomes; transfersomes; lecithin vesicles; ethosomes; water; surfactants, such as anionic, cationic, and nonionic surfactants; polyols; essential oils, and the like.

In some embodiments, compositions herein can include one or more mucoadhesive polymers. A “mucoadhesive polymer” as understood herein is a natural or synthetic macromolecule capable of adhering to mucosal tissue surfaces. In some embodiments, a polymer suitable for use herein can be carbopol, chitosan, sodium carboxymethyl cellulose (NaCMC), hydroxypropyl methylcellulose (HPMC), hydroxypropyl cellulose methylcellulose, poloxamer, polyoxyethylene, pluronic-poly(acrylic acid) copolymer, carbomer, chitosan, polyvinyl alcohol (PVA), poly(N-isopropylacrylamide) (PNiPAAm), methocel A4M, polymethacrylic acid and polyethylene glycol (P(MAA-g-EG), polyvinylacetal diethylamino acetate, or a combination thereof. In some examples, nasal formulations herein can include a mucoadhesive polymer in an amount of between about 0.1% (w/w) to about 25% (w/w), based on the total weight of the formulation.

In some embodiments, consistency of formulations can be created to generate topical gel-like formulations that include, an isolated Smad7, a recombinant Smad7, a fragment thereof, or fusion polypeptide thereof disclosed herein and at least one gelling agent. In some embodiments, a Smad7 molecule or fusion polypeptide or PTD fused polypeptide can have a concentration of about 1 ng/ml to about 2 mg/ml (e.g., about 1.0, 1.25, 1.5, 1.75, 2.0, 2.5, 3.0, 3.5, 4.0, 4.5, 5.0, 6.0, 7.0, 8.0, 9.0, 10, 15, 20, 25, 30, 35, 40, 45, 50, 55, 60, 65, 70, 75, 80, 85, 90, 95, 100, 110, 125, 150, 175, 200, 250, 300, 350, 400, 450, 500, 600, 700, 800, 900, 1000 ng/ml; or about 0.5, 0.75, 1.0, 1.25, 1.5, 1.75, 2.0, 2.5, 3.0, 3.5, 4.0, 4.5, 5.0, 6.0, 7.0, 8.0, 9.0, 10, 15, 20, 25, 30, 35, 40, 45, 50, 55, 60, 65, 70, 75, 80, 85, 90, 95, 100, 110, 125, 150, 175, 200, 250, 300, 350, 400, 450, 500, 600, 700, 800, 900, 1000, 1200, 1200, 1300, 1400, 1500, 1600, 1700, 1800, 1900, 2000 μg/ml) and about 0.01% to about 10% (e.g., about 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%) weight by volume (w/v) of at least one gelling agent. In some embodiments, compositions herein can be topical gel-like formulations that include an isolated Smad7, a recombinant Smad7, a fragment thereof, or fusion polypeptide thereof disclosed herein, at least one gelling agent, and can further include one or more of the additional agents and/or additives described herein.

In some embodiments, compositions disclosed herein can be formulated for local administration. As used herein, “local administration” refers to administration of one or more compositions disclosed herein directly to, in, or to the vicinity of, the region to be treated such as the mucosa or dermal region of a subject in need of such a treatment. In certain embodiments, “topical” or “local” delivery is directed to epidermal and dermal cells of the skin and scalp (including cells lining hair follicles), as well as mucosal cells of the mouth, salivary glands, GI tract, reproductive tract, the throat, and the like. In some embodiments, compositions herein can be formulations suitable for topical oral or GI application and/or for topical dermal application can include oral emulsions, creams, pastes, magmas, gels, swishes, lozenges, oral solutions, optical solutions, gums, etc., as are well known in the art. Any of these topical oral vehicles and/or dermal vehicles can be used in conjunction with the methods of the invention.

In some embodiments, compositions herein can provide a controlled, constant, sustained, instant, extended, or delayed rate of release of therapeutic protein (e.g., an isolated Smad7, a recombinant Smad7, a fragment thereof, or fusion polypeptide thereof) into epidermal and dermal cells of the skin and/or mucosal cells of the mouth, nose, salivary glands, throat, GI, reproductive tract and the like.

In some embodiments, compositions disclosed herein may be formulated by mixing a recombinant human Smad7, a fragment thereof, or fusion polypeptide thereof with a base-gel formulation. As used herein, a “base-gel formulation” refers to a gel formulation that includes at least one gelling agent disclosed herein and can further include any one or more agents and/or additives disclosed herein wherein the gel formulation is contemplated to be in a final formulation other than not having the therapeutic protein (e.g., a recombinant human Smad7, a fragment thereof, or fusion polypeptide thereof). In some embodiments, compositions disclosed herein can be formulated by mixing a recombinant human Smad7, a fragment thereof, or fusion polypeptide thereof with a base-gel formulation in a 10:1 ratio, a 9:1 ratio, a 8:1 ratio, a 7:1 ratio, a 6:1 ratio, a 5:1 ratio, a 4:1 ratio, a 3:1 ratio, a 2:1 ratio, or a 1:1 ratio or 1:2, 1:3, 1;4 or similar ratio. As an example, a 4:1 ratio herein refers to 4 parts by volume therapeutic protein (e.g., a recombinant human Smad7, a fragment thereof, or fusion polypeptide thereof) to 1 part by volume base-gel formulation.

In some embodiments, the compositions disclosed herein can be used in methods of preventing, treating, and/or ameliorating one or more diseases and conditions associated with excessive inflammation and epithelial ablation. As used herein, “epithelial ablation” refers to the disruption and/or removal of an epithelial tissue layer. As an example, epithelial ablation can be a result of massive apoptosis and blunted keratinocyte proliferation associated with diseases and/or conditions contemplated for treatment using the methods herein. In some embodiments, the compositions disclosed herein can be used in methods of preventing, treating, and/or ameliorating one or more diseases and conditions associated with epithelial ablation. Although not wishing to be bound by theory, proliferative and anti-apoptotic effects of Smad7 compositions herein can reduce excessive inflammation and/or epithelial ablation in diseases and/or conditions described herein by inhibiting NF-κB/a NF-κB signaling pathway, TGF-β1 and/or a TGF-β1 signaling pathway. Because inflammatory cells produce cytokines that further activate TGF-β and NF-κB, reduced TGF-β and NF-κB signaling reflects the direct antagonistic effect of Smad7 on these two pathways and the consequence of reduced inflammatory cytokines.

In some embodiments, the compositions disclosed herein can be used in methods of preventing, treating, and/or ameliorating one or more diseases and conditions associated with excessive inflammation and epithelial ablation such as a chronic wound or chronically infected tissue or non-healing tissue or wound. In some embodiments, wounds that do not heal within three months can be considered chronic. Chronic wounds can be delayed in one or more of the phases of wound healing. For example, chronic wounds often remain in the inflammatory stage for too long. In acute wounds, there is a precise balance between production and degradation of molecules such as collagen; in chronic wounds this balance is lost and degradation into epithelial ablation plays too large a role. In some embodiments, the compositions disclosed herein can be used in methods of preventing, treating, and/or ameliorating a chronic wound. Chronic wounds can be classified into four categories: venous ulcers, diabetic, pressure ulcers, and “other” where other includes wounds that do not fall into these categories that may be due to causes such as radiation poisoning or ischemia or similar side effect.

In some embodiments, methods of preventing, treating, and/or ameliorating one or more diseases and/or conditions associated with excessive inflammation and epithelial ablation can be preventing, treating, and/or ameliorating venous and/or arterial ulcers. Venous ulcers, which usually occur in the legs, account for about 70% to 90% of chronic wounds and mostly affect the elderly or subject with a condition. They are thought to be due to venous hypertension caused by improper function of valves that exist in the veins to prevent blood from flowing backward. Ischemia can be caused from dysfunction and ceasing of blood flow such as a constriction and, combined with reperfusion where the blood flow is restored can lead to injuries during this process such as necrosis, adverse remodeling and other tissue damage and wounds.

In some embodiments, methods of preventing, treating, and/or ameliorating one or more diseases and/or conditions associated with excessive inflammation and epithelial ablation can be preventing, treating, and/or ameliorating diabetic ulcers or necrosis are disclosed. Diabetics have a 15% higher risk for amputation than the general population due to chronic ulcers. Diabetes causes neuropathy, which inhibits nociception and the perception of pain. Diabetic patients may not initially notice small wounds to legs and feet and may therefore fail to prevent infection or repeated injury. Further, diabetes causes immune compromise and damage to small blood vessels, preventing adequate oxygenation of tissue, which can cause chronic wounds.

In some embodiments, methods of reducing the risk of, preventing, treating, and/or ameliorating one or more diseases and/or health conditions associated with excessive inflammation and epithelial ablation can be reducing the risk of onset, preventing, treating, and/or ameliorating pressure ulcers. Another leading type of chronic wounds is pressure ulcers, which usually occur in people with conditions such as paralysis that inhibit movement of body parts that are commonly subjected to pressure such as the heels, shoulder blades, and sacrum. Pressure ulcers are caused by ischemia that occurs when pressure on the tissue is greater than the pressure in capillaries, and thus restricts blood flow into the area. Muscle tissue, which needs more oxygen and nutrients than skin does, shows the worst effects from prolonged pressure. As in other chronic ulcers, reperfusion injury damages tissue.

In some embodiments, chronic wounds, or chronically infected tissue or tissue damage to be treated with compositions disclosed herein can affect one or more of: only the epidermis, only the dermis, the epidermis and the dermis, and/or tissues and/or all the way to the fascia of the subject to be treated. In some embodiments, chronic wounds to be treated with compositions disclosed herein can be a result of anything that causes an acute wound, such as surgery or accidental trauma, or as a result of systemic infection, vascular, immune, or nerve insufficiency such as reduced or inhibited blood flow, or comorbidities such as neoplasia or metabolic disorders.

In some embodiments, the compositions disclosed herein can be used in methods of reducing the risk of, preventing, treating, and/or ameliorating one or more toxic effects of a cancer therapy, such as with chemotherapeutic agents or radiation. In some embodiments, methods herein can protect cells, preferably non-neoplastic cells, from damage during cancer chemotherapy or radiotherapy. In accordance with some embodiments herein, methods of protecting cells from damage during cancer chemotherapy or radiotherapy can include administering to a population of epithelial cells a composition as described above, for a time and in an amount effective to protect the non-neoplastic cells from damage during radiotherapy or cancer chemotherapy. In some embodiments, methods of protecting cells from damage during cancer chemotherapy or radiotherapy can prevent one or more side effects of the treatment (e.g., psoriasis, dermatitis, ulceration, oral mucositis). In some embodiments, methods herein can treat cells damaged during cancer chemotherapy or radiotherapy. In accordance with some embodiments herein, methods of treating cells from damage during cancer chemotherapy or radiotherapy can include administering to a population of epithelial cells a composition as described above, for a time and in an amount effective to reverse and/or attenuate cellular damage caused during radiotherapy or cancer chemotherapy. In some embodiments, methods of treating cells from damage during cancer chemotherapy or radiotherapy can reverse and/or attenuate one or more side effects of the treatment (e.g., psoriasis, dermatitis, ulceration, oral mucositis). In some embodiments, methods of treating oral mucositis can slow and/or prevent progression of oral mucositis from an initial less severe state (e.g., stage 1-3) to a more severe state (e.g., stage 4-5). In general, more severe states are those that risk needing to use feeding tubes and/or hospitalization. Table 2 provides a description of suitable methods of grading oral mucositis for use herein when assessing treatment regimens using compositions and formulations disclosed herein.

TABLE 2

| | Grade 0 | Grade 1 | Grade 2 | Grade 3 | Grade 4 |
|---|---|---|---|---|---|
| WHO | None | Soreness with | Erythema, | Ulcers, liquid | Alimentation |
| | | erythema | ulcers, can eat | diet only | not possible |
| | | | solids | | |
| RTOG | None | Erythema of | Patchy reaction | Confluent | Necrosis or |
| | | mucosa | <1.5 cm, | reaction > 1.5 | deep |
| | | | noncontiguous | cm, contiguous | ulceration, ± |
| | | | | | bleeding |
| NCI CTC | None | Painless ulcers, | Painful | Painful | Severe |
| | | erythema, or | erythema, | erythema, | ulcerations or |
| | | mild soreness | edema, or | edema, or | requires |
| | | in absence of | ulcers, but can | ulcers, | parental/ |
| | | lesions | eat/swallow | requiring IV | enteral |
| | | | | hydration | nutritional |
| | | | | | support or |
| | | | | | prophylactic |
| | | | | | intubation |
| OMAS | Normal/ | Not severe/ | Severe | NA | NA |
| Ulceration/ | Normal | <1 sq cm | 1-3 sq cm | >3 sq cm | NA |
| eryrthema | | | | | |

OM: oral mucositis;
WHO: World Health Organization;
RTOG: Radiation Therapy Group;
±: with or without;
NCI CTC: National Cancer Institute Common Toxicology Criteria;
NA: not applicable.

In some embodiments, methods of protecting cells from damage during cancer chemotherapy or radiotherapy and/or methods of treating cells damaged during cancer chemotherapy or radiotherapy can be administered as disclosed herein at appropriate intervals, before, during, or after a regimen of chemotherapy and/or radiotherapy, during and after particularly for chemotherapy regimens. The appropriate interval in a particular case would normally depend on the nature of the chemotherapy or radiotherapy and the cell population targeted for protection.

In some embodiments, methods herein can include administering to a subject in need one or more of the compositions disclosed herein. A suitable subject includes a human, a livestock animal, a companion animal, a wild animal, a lab animal, or a zoological animal. Non-limiting examples of suitable livestock animals may include pigs, cows, goats, sheep, llamas, and alpacas. Other animals include horses such as performance horses. In yet another embodiment, the subject may be a companion animal. Non-limiting examples of companion animals may include pets such as dogs, cats, rabbits, and birds. In yet another embodiment, the subject may be a zoological animal or wild animal. As used herein, a “zoological animal” refers to an animal that may be found in a zoo. Such animals may include non-human primates, large cats, wolves, and bears. In some embodiments, the subject is a mammal. In some embodiments, the subject is a human.

In some embodiments, a subject in need one or more of the compositions disclosed herein can have one or more side effects of a chronic wound. In some embodiments, a subject in need one or more of the compositions disclosed herein can have one or more side effects of venous ulcers, diabetic, pressure ulcers, or a combination thereof.

In some embodiments, a subject in need one or more of the compositions disclosed herein can have one or more side effects of a treatment for cancer such as chemotherapy, radiotherapy, and/or a topical anti-tumor agent. In some embodiments, one or more side effects of a treatment for cancer such as chemotherapy, radiotherapy, and/or a topical anti-tumor agent can be psoriasis, dermatitis, ulceration, oral mucositis, or any combination thereof. In some embodiments, a subject in need one or more of the compositions disclosed herein can have one or more symptoms associated with a disease or the condition wherein the one or more symptoms associated with the disease, or the condition comprises inflammation, necrosis, or a combination thereof.

In some embodiments, methods of preventing, treating, and/or ameliorating one or more side effects of a chronic wound or chronically infected tissue (e.g., venous ulcers, diabetic, pressure ulcers) can include administering an effective amount of an isolated Smad7, a recombinant Smad7, a fragment thereof, or fusion polypeptide thereof. In certain embodiments, the Smad7 is a human Smad7 polypeptide or other mammalian Smad7. In some embodiments, methods of reducing the risk of onset, preventing, treating, and/or ameliorating one or more side effects of a treatment for cancer such as chemotherapy, radiotherapy, and/or a topical anti-tumor agent can include administering an effective amount of formulation containing a recombinant human Smad7, a fragment thereof, or fusion polypeptide thereof and a gelling agent disclosed herein. “An effective amount” as used herein refers to the amount of each therapeutic protein (e.g., an isolated, a recombinant Smad7, a fragment thereof, or fusion polypeptide thereof) required to confer therapeutic effects on the subject, either alone or in combination with one or more other active agents. Effective amounts can vary, as recognized by those skilled in the art, depending on route of administration, excipient usage, and co-usage with other active agents.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In some embodiments, dosages of topical formulations as described herein can be determined empirically in individuals who have been given one or more administration(s) of the formulation. Generally, for administration of any of the topical formulations described herein, an initial candidate dosage of a recombinant human Smad7 a fragment thereof, or fusion polypeptide thereof can be about 0.01 μg/day to about 50.0 μg/day (e.g., 0.01, 0.1, 0.5, 1.0, 2.0, 5.0, 10, 20, 30, 40, 50 μg/day) or more depending on the factors mentioned above. For repeated administrations over several days or longer, depending on the condition, the treatment can be sustained until a desired suppression of symptoms occurs or until sufficient therapeutic levels are achieved to alleviate a target disease or disorder, or a symptom thereof. In other embodiments, dosage regimens can be useful, depending on the pattern of pharmacokinetic decay that the practitioner wishes to achieve. For example, dosing from about one to seven times a week is contemplated. In some embodiments, dosing ranging from about 0.01 μg to about 50 μg per day can be used depending on the condition to be treated, the mode of administration and the subject to be treated, for example. In some embodiments, dosing frequency is three times a day or more, two times a day, once every day, every other day, twice a week, once per week, every 2 weeks, every 4 weeks, every 5 weeks, every 6 weeks, every 7 weeks, every 8 weeks, every 9 weeks, or every 10 weeks; or once every month, every 2 months, or every 3 months, or longer or whenever psoriasis, dermatitis, ulceration, and/or oral mucositis improves. In some embodiments, dosing ranging from about 0.01 μg to about 50 μg per day can be used at a dosing frequency of about three times a day or more, two times a day, once every day, every other day, twice a week, or once per week until the health conditions is treated or prevented such as reduced psoriasis, dermatitis, ulceration, a wound has healed and/or oral mucositis improves or is eliminated, inflammation is reduced, or other desired effect. In certain embodiments, treatments can vary for a specific condition and/or subject being treated. In certain embodiments, a treatment for oral Mucositis or mucositis can be about 0.1 to about 20 or about 0.5-8.0 ug per treatment of active agent. In other embodiments, a treatment for psoriasis can be from about 0.1 to about 20 or about 0.8-8.0 ug 0.8-8 ug per treatment of active agent. In other embodiments, a treatment for a wound (e.g., diabetic, or non-diabetic wound or surgical wound etc.) can be from about 0.1 to about 20; or about 0.8-8.0 ug per treatment of active agent. In yet other embodiments, a treatment for radiodermatitis in a subject can be from about 0.5 to about 20.0 or about 5.0 to about 10.0 ug per treatment of active agent. In accordance with these embodiments, an active agent of formulations and treatments disclosed herein can include one or more SMAD7 constructs, fusion polypeptides or fragments of a SMAD7 polypeptide as disclosed herein. It is noted herein that the progress of these therapy is easily monitored by conventional techniques and assays known in the art. In some embodiments, improvement in wound healing, psoriasis, dermatitis, ulceration, and/or oral mucositis can be assessed by routinely monitoring gross appearance according to methods known in the art or other methods known in the art. In some embodiments, improvement in wound healing, psoriasis, dermatitis, ulceration, and/or oral mucositis can be assessed by routinely monitoring serum levels of one or more inflammatory cytokines according to methods known in the art.

Any mode of administration of the formulations and compositions is contemplated herein. In some embodiments, the mode is oral, intravenous, intranasal, subcutaneous, topical such as by a salve or gel or other appropriate mode of administration. In other embodiments, a pad, bandage, adhesive, microbeads or microparticles, or other applicable delivery device can be embedded with formulations disclosed herein for improved efficacy, reduce exposure or the like.

In certain embodiments, a mode of administration of the formulations and compositions can be by nasal aerosol or inhalation. Such formulations and compositions can be prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents. In some embodiments, formulations and compositions herein for inhalation and/or insufflation can include solutions and suspensions in pharmaceutically acceptable, aqueous or organic solvents, or mixtures thereof, and powders. In some embodiments, liquid or solid formulations and compositions herein can contain one or more suitable pharmaceutically acceptable excipients as set out above. In some embodiments, formulations and compositions herein can be administered by an oral or a nasal respiratory route for local or systemic effect. In some embodiments, formulations and compositions herein can be suitable for intranasal administration or inhalation, such as delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoro-ethane, a hydrofluoroalkane, carbon dioxide or other suitable gas. In some embodiments, having a pressurized aerosol, a dosage unit can be determined by providing a valve to deliver a metered amount.

In certain embodiments, a mode of administration of the formulations and compositions can be by topical administration. In some embodiments, formulations and compositions can be made in the form of an article such as a tape, a patch, a sheet, a dressing or any other form known to those skilled in the art for a topical administration. In accordance with these embodiments, an active ingredient (e.g., SMAD7 constructs, fusion polypeptides or fragments of a SMAD7 polypeptide as disclosed herein) can be introduced into a article for topical administration in different forms (solid, in solution, in dispersion) and/or it can also be microencapsulated.

In certain embodiments, the one or more symptoms associated with the disease, or the health condition include, but is not limited to, inflammation, necrosis, oral mucositis, psoriasis, or a combination thereof. In some embodiments, oral mucositis, radiodermatitis, atopic dermatitis, allergic dermatitis, psoriasis, gastrointestinal mucosal complications, chronic (or diabetic) wounds, traumatic or surgically induces wounds (e.g., excisions, acute wounds post-surgery or post trauma), scarring, autoimmune related oral ulcer, periodontal inflammation, or a combination thereof can be a secondary result from one or more cancer treatments or other health condition and are contemplated to be treated by formulations disclosed herein.

In other embodiments, the present disclosure also provides kits for use in delivering therapeutic proteins (e.g., a recombinant human Smad7, a fragment thereof, or fusion polypeptide thereof) to a target site or for use in preventing, treating, and/or ameliorating one or more side effects of a chronic wound (e.g., venous ulcers, diabetic, pressure ulcers) and/or cancer chemotherapy or radiotherapy (e.g., psoriasis, dermatitis, ulceration, oral mucositis). Such kits can include one or more containers comprising any of the compositions described herein.

In some embodiments, kits herein can include instructions for use in accordance with any of the methods described herein. The included instructions can include a description of administration of the composition for delivering the therapeutic proteins (e.g., an isolated, a recombinant Smad7, a fragment thereof, or fusion polypeptide of any Smad7 molecule thereof) therein or for preventing, treating, and/or ameliorating one or more side effects of cancer chemotherapy or radiotherapy can prevent one or more side effects of the treatment (e.g., psoriasis, dermatitis, ulceration, oral mucositis) according to any of the methods described herein. The kit can further include a description of selecting an individual suitable for treatment based on identifying whether that individual has, is suspected of having, or is at risk for damaging side effects of a cancer chemotherapy or radiotherapy and/or impaired wound healing of a chronic wound.

The instructions relating to the use of the compositions described herein, which can include recombinant human Smad7, a fragment thereof, or fusion polypeptide thereof and a gelling agent, generally include information as to dosage, dosing schedule, and route of administration for the intended treatment. In some embodiments, containers herein can be unit doses, bulk packages (e.g., multi-dose packages) or sub-unit doses. In some embodiments, containers herein can be syringes containing the compositions disclosed herein. In some embodiments, a label or package insert can indicate that the composition as disclosed herein can be used for delivering therapeutic proteins to a target site (e.g., skin, oral mucosa). Instructions can be provided for practicing any of the methods described herein. Instructions supplied in the kits herein can typically be written instructions on a label or package insert (e.g., a paper sheet included in the kit), but machine-readable instructions (e.g., instructions carried on a magnetic or optical storage disk).

In some embodiments, kits as described herein are in suitable packaging. Suitable packaging includes, but is not limited to, vials, bottles, jars, flexible packaging (e.g., sealed Mylar or plastic bags), and the like. In some embodiments, packages can be included in kit herein for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). In some embodiments, kits disclosed herein having one or more containers can also have a sterile access port (for example the container may be an intravenous solution bag or a vial having a stopper pierceable by a hypodermic injection needle).

The kits described herein can optionally provide additional components such as buffers and interpretive information. Kits herein can include a container and a label or package insert(s) on or associated with the container. In some embodiments, the present disclosure provides articles of manufacture comprising contents of the kits described above.

Below is an exemplary sequence listing. Other SMAD7 fragments and fusion polypeptides are contemplated of use in formulations and compositions disclosed herein.

SEQUENCE LISTING

| SEQ ID NO | Description |
|---|---|
| 1 | Human SMAD7 (UniProtKB/Swiss-Prot: O15105.1) |
| 2 | Human SMAD7 (NCBI Reference Sequence: NM_005904.4) |
| 3 | Human SMAD7 (aa 203-426) |
| 4 | Human SMAD7 (aa 259-426) |
| 5 | Human SMAD7 (aa 203-217) |
| 6 | TAT (47-57) |
| 7 | TAT (48-60) |
| 8 | TAT (49-57) |
| 9 | TAT (48-57) |
| 10 | Antp-43-58 |
| 11 | hCT (9-32) |
| 12 | Pep-1 |
| 13 | MAP |
| 14 | Transportan |
| 15 | pVEC |
| 16 | Azurin p28 (50-77) |
| 17 | VP22 |
| 18 | ARF (1-22) |
| 19 | VT5 |
| 20 | C105Y |
| 21 | FGF |
| 22 | Pep-7 |
| 23 | PFV |
| 24 | Vectocell |
| 25 | HA |
| 26 | GST cleavage site |

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of embodiments of the inventions.

### Example 1

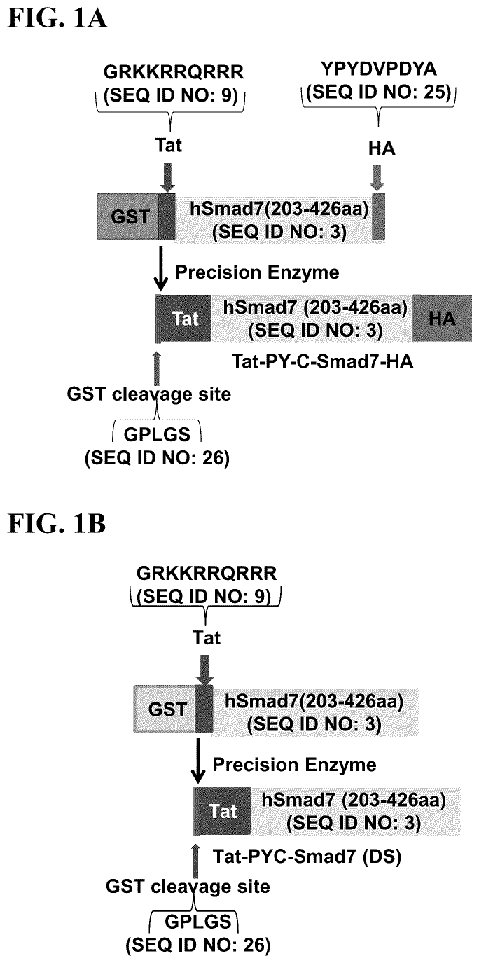

In one exemplary method, Smad7 fusion proteins were prepared in a similar manner to the methods described in Han et al., Nat Med 19, 421-428 (2013), the disclosure of which is incorporated herein in its entirety. In brief, a recombinant Tat-tagged protein containing amino acids 203-426 of human Smad7 (Tat-PYC-Smad7) as a fusion protein was produced. In this example, constructs containing amino acids 203-426 of human Smad7 cDNA with a 5′-Tat-tag sequence encoding GRKKRRQRRR (SEQ ID NO. 8) and a 3′ HA-tag sequence encoding YPYDVPDYA (SEQ ID NO. 24) was generated (FIG. 1A) and cloned into a pGEX-6p-1 protein expression vector (New England Biolabs) to make a GST-Tat-PYC-Smad7 fusion protein. GST-Tat-PYC-Smad7 was transformed into BL-21 Star E. coli to produce GST-Tat-PYC-Smad7 protein. Tat-PYC-Smad7 was cleaved from GST at GPLGS (SEQ ID NO. 26) with PreScission protease (FIG. 1B) followed by either size exclusion chromatography (SEC) or heparin affinity chromatography to purify Tat-PYC-Smad7 protein.

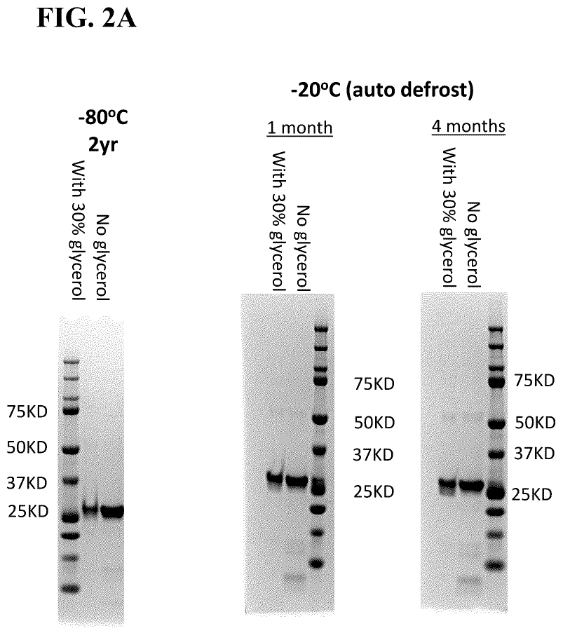

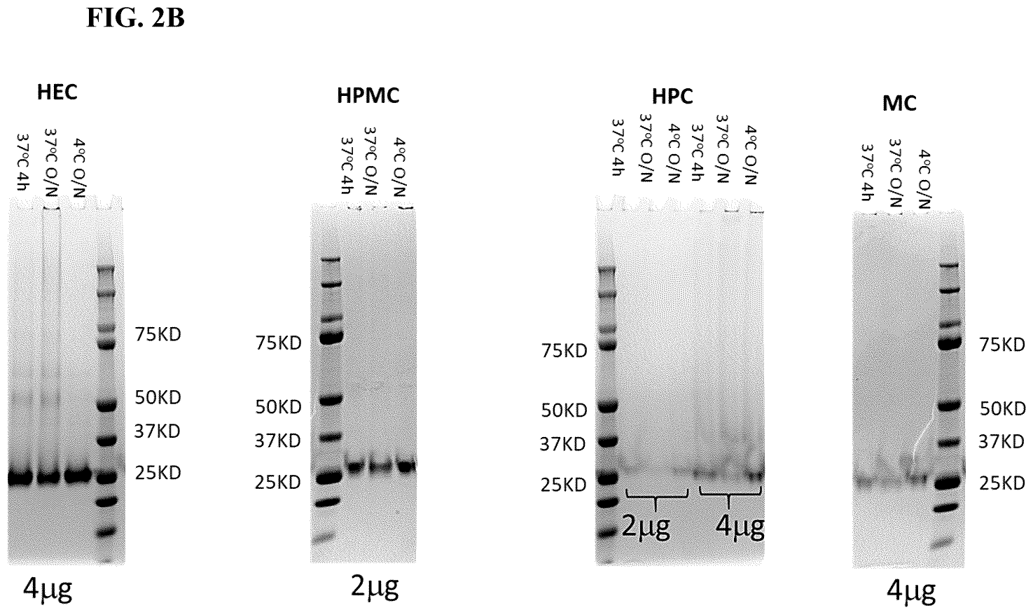

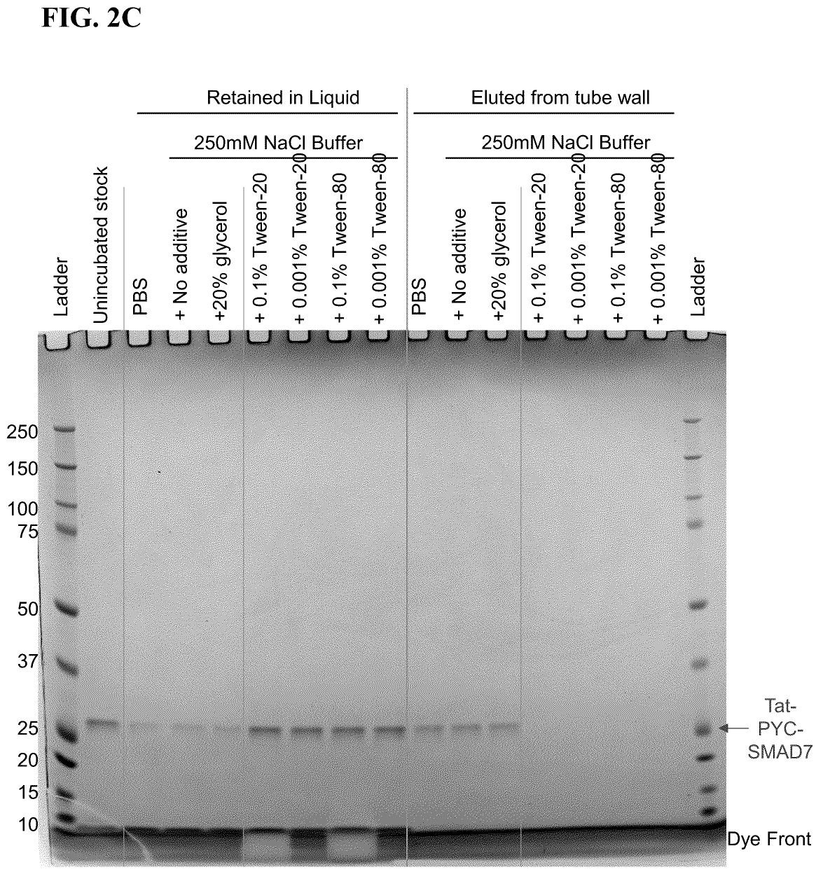

In an exemplary method, Tat-PYC-Smad7 was formulated in PBS with or without 30% glycerol to assess stability and retention of the fusion protein over time at −80 C and in a −20 C freezer that included auto-defrost. A non-reducing SDS-PAGE gel of samples following 2 years at −80 C and 1 month and 4 months at −20 C (autodefrost) showed that the presence of 30% glycerol in the Tat-PYC-Smad7 formulation enhanced Tat-PYC-Smad7 stability (reduced degradation) (FIG. 2A).

In an exemplary method, Tat-PYC-Smad7 was formulated in PBS with or without 20% glycerol and with or without Polysorbate 20 or Polysorbate 80 to assess binding of Tat-PYC-Smad7 to container walls. Storage of Tat-PYC-Smad7 in PBS in glass, polypropylene or polystyrene containers resulted in reduced Tat-PYC-Smad7 in the liquid phase and Tat-PYC was previously detected bound to the container walls (resistant to PBS rinsing but eluted with sample buffer). Tat-PYC-Smad7 stock was incubated overnight in PBS (negative control), SEC buffer (250 mM NaCl) or SEC buffer containing 20% glycerol, Tween 20 or Tween 80 and separated on a non-reducing SDS-PAGE gel (FIG. 2C). The data showed that the presence of Tween 20 (polysorbate 20) or Tween 80 (polysorbate 80) was required to prevent loss of Tat-PYC-Smad7 from solution and binding to container wall.

In an exemplary method, Tat-PYC-Smad7 in PBS was formulated with gelling agents HPC, HPMC, HEC, and MC to develop a Smad7 formulation for use as an oral gel (e.g., for the treatment or prevention of oral mucositis) or as a topical gel (e.g., for the treatment of radiodermatitis or wound healing). The formulations of Tat-PYC-Smad7 (2 μg and 4 μg) were separated by non-reducing SDS-PAGE following storage at 4° C. overnight, 37° C. for 4 hours, or 37° C. overnight (FIG. 2B). HEC and HPMC appeared to have similar stability profiles in these conditions, and were more stable in accelerated stability (37° C.) than HPC and MC. Because HEC and HPC do not foam and have the desired viscosity and “feel”, they were further tested in functional tests.

In an exemplary method, a formulation was prepared for topical administration (e.g., local, oral mucosa, topical, or dermal application) of formulated Tat-PYC-Smad7. In this exemplary method, hydroxypropyl cellulose (HPC) gel (1% Klucel HF, Ashland; 0.1 to 5% is also contemplated to work) containing 1×PBS (137 mM NaCl, 2.7 mM KCl, 8 mM Na₂HPO₄, and 2 mM KH₂PO₄; it is contemplated that 15% increase or decrease in these concentrations would also work for formulations disclosed herein) and 15% glycerol (about 5 to about 25% is also contemplated to work) was used as the base of an oral gel. Tat-PYC-Smad7 was formulated into the oral gel by mixing a 1:1. 2:1 or 3:1 ratio of fusion polypeptide stock to the base gel to obtain a final concentration of 0.5-100 μg/ml Tat-PYC-Smad7. The fusion polypeptide stock is 0.05-2.0 mg/ml Tat-PYC-Smad7 in 250 mmol NaCl, pH 7.0-8.0, 0.0001% to 0.1% Tween-80 or Tween-20, and 15-30% glycerol.

In another exemplary method, other topical formulations of Tat-PYC-Smad7 were prepared. In this exemplary method, a cellulose-base gel was made from either 0.5-0.7% Klucel (HPC) or 0.5-1% Natrosol G250 (HEC) in addition to USP/NF grade excipients as provided in Table 3. The base gel was then hydrated and autoclaved before adding Tat-PYC-Smad7 in a 1:1, 2:1, 3:1 or 4:1 dilution into the cellulose-base gel in order to reduce contamination and enhance preservation (stability) of the construct, for example. The gel was combined with Tat-PYC-Smad7 at other ratios depending on a desired viscosity and/or the Tat-PYC-Smad7 dose desired. In this example, the resulting formulations ranged from about 7.0 to 9.0.

TABLE 3

| | Concentration or Mass per Dosage Form (e.g., |
|---|---|
| Ingredient | per mL or per tablet) |
| Tat-PYC-Smad7 | 0.5 μg/ml or 20 μg/ml |
| Gelling agent | 0.5-0.7% Klucel (HPC), Ashland or 0.5-1% |
| | Natrosol G250 (HEC), Ashland |
| Glycerol, protein stabilizer | 15%-30% |
| Sodium chloride, sodium | 150 mM Sodium chloride, 10-25 mM sodium |
| phosphate, equivalent to body fluid | phosphate pH 7.4 ± 0.5 |
| Tween-80, surfactant | 0.0001%-0.1%, also prevents protein sticking onto |
| | polypropylene surface of the container |

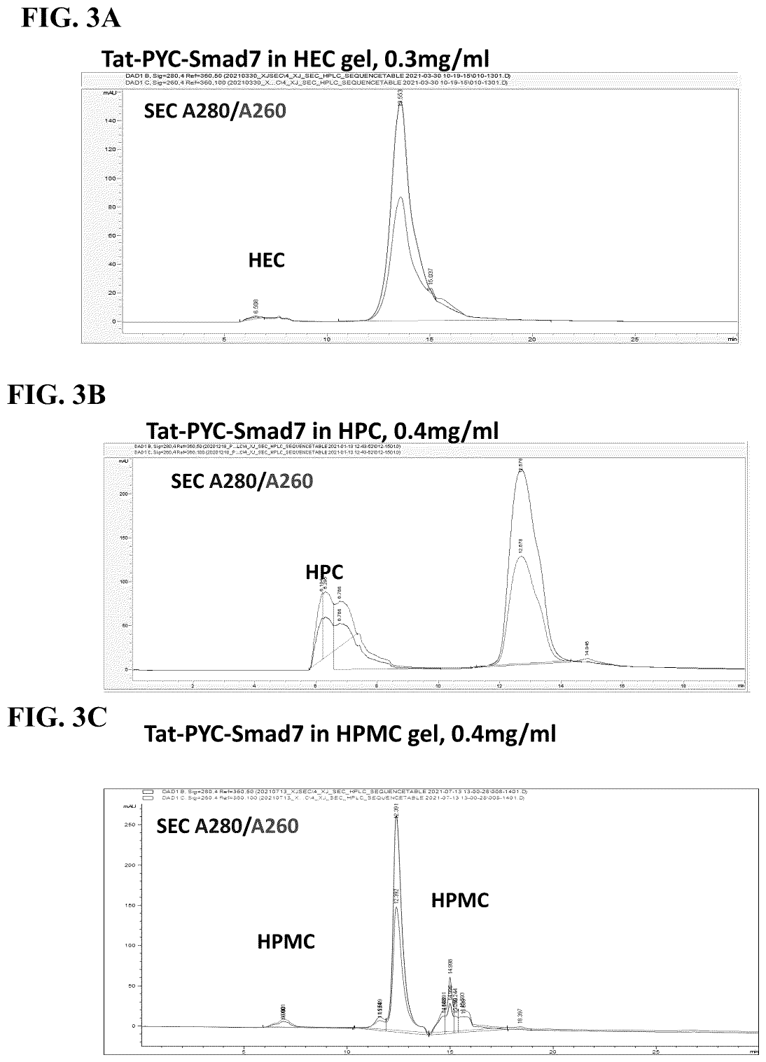

In another exemplary method, Tat-PYC-Smad7 gel formulations were characterized by size-exclusion chromatography (SEC). FIGS. 3A-3C illustrate that the SEC analytical profiles of Tat-PYC-Smad7 in HPC, HPMC and HEC. Both HPC and HPMC showed significant additional peaks in SEC, a confounding factor for gaining FDA approval. The HEC profile was surprisingly the cleanest, with only a minor band indicating the presence of HEC, and so is a preferred gelling agent. These peaks were observed in the presence or absence of Tat-PYC-Smad7. For this reason, as well as others, the gelling agent HEC in certain formulations is preferred over HPC and other agents such as HPMC are likely to be of use.

In another exemplary method, SEC of Tat-PYC-Smad7 gel formulations also identified salt and pH effects. Formulations having 1) 500 mM NaCl, 50 mM NaPO4, pH 7.8 or 2) 250 mM NaCl, 25 mM NaPO4, pH 7.5 had comparable SEC profiles. In Tat-PYC-Smad7 gel formulations having a pH below 7 (e.g., a formulation having 150 mM NaCl, 25 mM NaPO4, pH 6.5, and a formulation having 150 mM NaCl, 25 mM NaPO4, 1 mM DTT, pH 6.0), SEC profiles were indicative of aggregation. (Data not shown but available on request.)

Because the effective dose of Tat-PYC-Smad7 was expected to be low, its concentration ranges from about 0.5-2 μg/mL in the gel, Tat-PYC-Smad7 content and/or purity was also assessed by SDS-PAGE performed under non-reducing conditions with silver staining in another exemplary method. Tat-PYC-Smad7 and BSA in HPC matrix or HEC matrix were separated by non-reducing SDS gel and silver stained. In the HPC matrix, the proteins had a noticeably lower intensity compared to the same amount of protein in the HEC matrix and HEC matrices appeared to be superior. The cause of this reduction of dye binding in the presence of HPC was not protein specific, as it was also observed with BSA. This phenomenon was not observed in HEC. (Data not shown but available on request.)

### Example 2

In another exemplary method, an oral mucositis model was established in dogs to assess the use of a Smad7-based biologic for treating oral mucositis in a clinically relevant setting and to analyze molecular targets of Smad7.

IMRT-induced oral mucositis in dogs. First, in an exemplary method herein, IMRT-induced oral mucositis was established in dogs in a clinically relevant setting. Oral mucositis (OM) is a term for the erythematous, inflammatory, and ulcerative lesions that occur in the mucosal lining of the mouth and pharynx secondary to cytotoxic cancer therapy. Up to 90% of patients with head and neck cancer (HNC) receiving radiotherapy (RT) will develop OM. Intensity-modulated radiation therapy (IMRT) is an advanced type of radiation therapy used to treat cancer and noncancerous tumors wherein a common, painful side effect is OM. To establish IMRT-induced oral mucositis in dogs herein, a total of 10 beagles of both genders (male and female) and ages ranging from 2.5 to 4 years old were used in the clinical protocol outlined in Table 4.

TABLE 4

| | | | | | | |
|---|---|---|---|---|---|---|
| Sun | Mon | Tues | Wed | Thu | Fri | Sat |

| | | | | | |
|---|---|---|---|---|---|
| | Dogs Arrive | | Lab work, | | |
| | | | pre-biopsy | | |
| Week 1 | RT | RT | RT | RT | RT |
| Week 2 | Dose X: Oral | Dose X: Oral | Dose X: Oral | Dose X: Oral | Dose X: Oral |
| Tat-PYC- | Treatment | Treatment | Treatment | Treatment | Treatment |
| Smad7 | VRTOG score | VRTOG score | VRTOG score | VRTOG score | VRTOG score |
| treatment | | | | | |
| Week 3 | Dose X: Oral | Dose X: Oral | Dose X: Oral | Dose X: Oral | Dose X: Oral |
| Tat-PYC- | Treatment | Treatment | Treatment | Treatment | Treatment |
| Smad7 | VRTOG score | VRTOG score | VRTOG score | VRTOG score | Biopsy, Lab work |
| treatment | | | | | VRTOG score |
| Week 4 | VRTOG score | VRTOG score | VRTOG score | VRTOG score | VRTOG score |

Prior to radiotherapy, standard laboratory assessments (e.g., a complete blood count (CBC) panel measuring red blood cell count, hemoglobin, hematocrit, MCV (mean corpuscular volume), MCH (mean corpuscular hemoglobin), MCHC (mean corpuscular hemoglobin concentration), RDW (red blood cell distribution), white blood cell count, differential count, platelet count; a biochemistry panel measuring fasting glucose, uric acid, BUN (blood urea nitrogen), creatinine, BUN/creatinine ratio, eGFR (estimated glomerular filtration rate), sodium, potassium, chloride, calcium, phosphorus, iron, total protein, albumin, globulin, albumin/globulin ratio, bilirubin, alkaline phosphatase, LDH (lactate dehydrogenase), AST (aspartate aminotransferase), ALT (alanine transaminase), total cholesterol, triglycerides, HDL cholesterol, LDL cholesterol (calc.), VLDL cholesterol (calc.), total cholesterol/HDL ratio, estimated CHD risk; and urinalysis measuring glucose, urinary protein, ketones, blood, leukocyte esterase, nitrite, bilirubin, urobilinogen, pH) and a full physical examination were performed.

A 6-mm pre-treatment biopsy from the IMRT mapped area was performed in an exemplary method herein. Each dog received IMRT irradiation daily for 5 days in week 1. Ionizing radiation doses ranging from 4 gray (Gy) to 6 Gy×5 fractions (20-30 Gy) (600 cGy/min) to maxilla (with exception of one mandible mucosa) were tested in the region extending from the incisors to the caudal aspect of the canine teeth and selected 5 Gy×5 (25 Gy) to maxillary mucosa for treatment, the total irradiated area was about 23.1 cm² (range, 20.6-38.1 cm²).

Evaluation of oral mucositis severity. In an exemplary method herein, gross appearance of oral mucositis was scored daily based on VRTOG (Veterinary Radiation Therapy Oncology Group) criteria described in Table 5.

TABLE 5

| Organ/Tissue | 0 | 1 | 2 | 3 |
|---|---|---|---|---|
| Skin/hair | no change | erythema, dry | patchy moist | confluent moist |
| | over | desquamation, | desquamation | desquamation with |
| | baseline | alopecia/epilation | without edema | edema and/or |
| | | | | ulceration, necrosis, |
| | | | | hemorrhage |
| Mucous | no change | injection without | patchy mucositis | confluent fibrinous |
| membrane/oral | over | mucositis | with patient | mucositis |
| cavity | baseline | | seemingly painfree | necessitating |
| | | | | analgesia, ulceration, |
| | | | | hemorrhage, |
| | | | | necrosis |
| Eye | no change | mild conjunctivitis | KCS requiring | severe keratitis with |
| | over | and/or scleral | artificial tears, | corneal ulceration |
| | baseline | injection | moderate | and/or loss of vision, |
| | | | conjunctivitis or iritis | glaucoma |
| | | | necessitating therapy | |
| Ear | no change | mild external otitis | moderate external | severe external otitis |
| | over | with erythema, | otitis requiring | with discharge and |
| | baseline | pruritis 2° to dry | topical medication | moist desquamation |
| | | desquamation not | | |
| | | requiring therapy | | |
| Lower GI | no change | change in quality of | diarrhea requiring | diarrhea requiring |
| | over | bowel habits not | parasympatholytic | parenteral support, |
| | baseline | requiring | medications, rectal | bloody discharge |
| | | medication, rectal | discomfort requiring | necessitating |
| | | discomfort | analgesia | medical attention, |
| | | | | fistula, perforation |
| Genitourinary | no change | change in frequency | change in frequency | gross hematuria or |
| | over | of urination not | of urination | bladder obstruction |
| | baseline | requiring medication | necessitating | |
| | | | medication | |
| CNS | no change | minor neurologic | neurologic findings | serious neurologic |
| | over | findings not | necessitating more | impairment such as |
| | baseline | necessitating more | than prednisone | paralysis, coma, |
| | | than prednisone | therapy | obtunded |
| | | therapy | | |
| Lung | no change | alveolar infiltrate; | dense alveolar | dyspnea |
| | over | cough requiring no | infiltrate; cough | |
| | baseline | treatment | requiring treatment | |

General health was also monitored in the animals. At the time of development of grade 3 mucositis, dogs were treated with gabapentin (5-10 mg/kg orally every 8-12 hours) +/−amantadine (2-3.5 mg/kg orally every 24 hours). Full physical examinations were performed daily until their VRTOG score went back to 0, typically at the end of week 4. Post treatment biopsy was performed 30 minutes after the last drug treatment on week 3. Post-treatment biopsies were fixed in 10% formalin, embedded in paraffin, and cut in 5 μm sections. Histology of H&E (hematoxylin and eosin) stained slides was performed independently by two treatment-blinded pathologists.

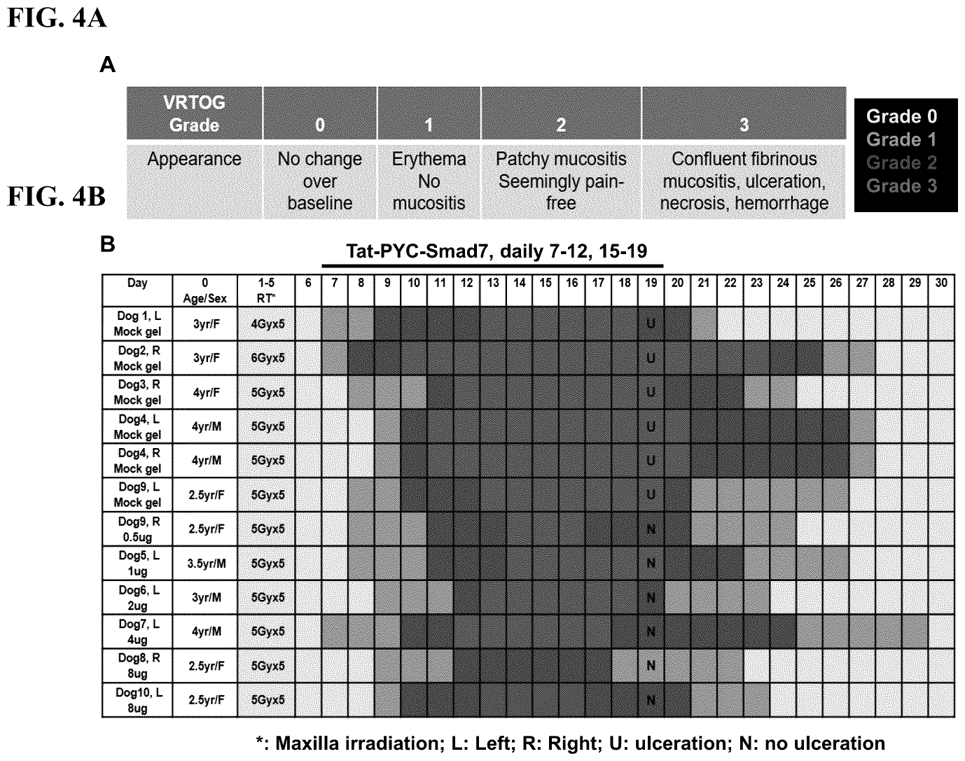

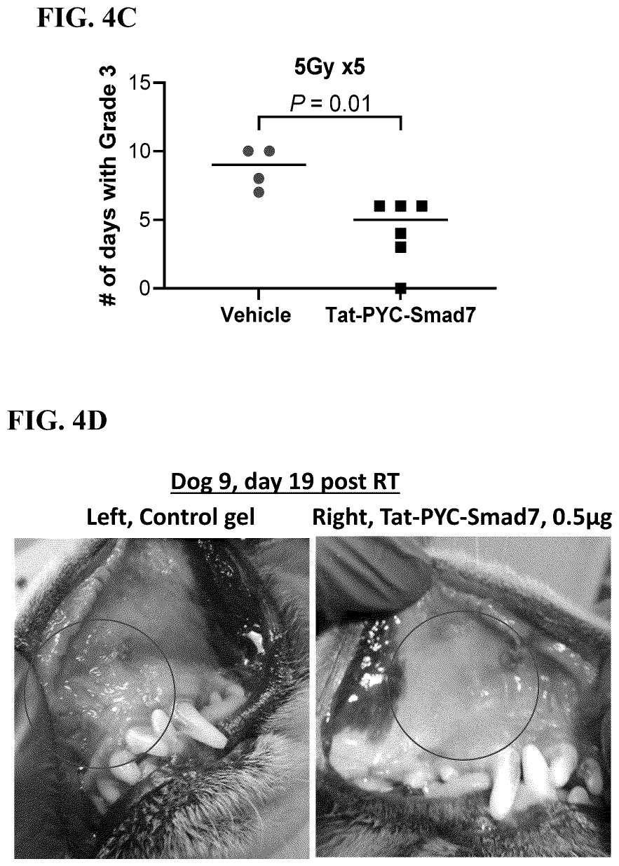

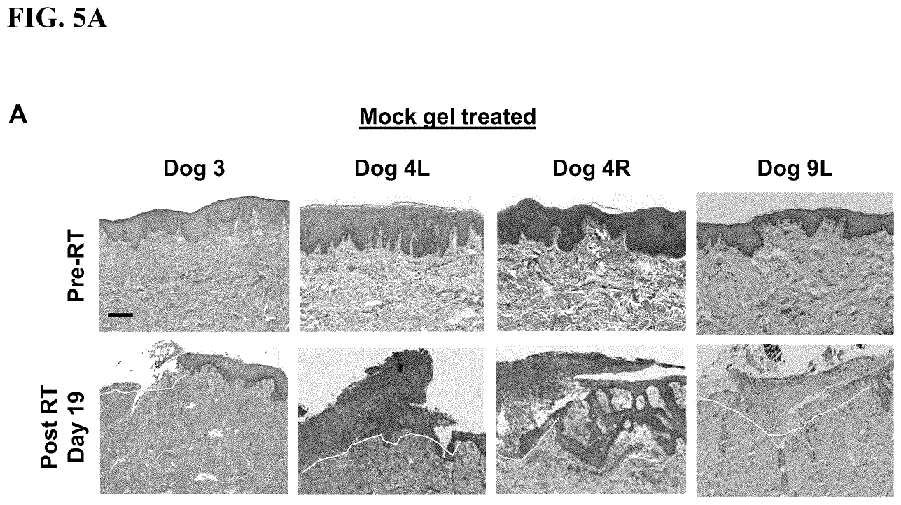

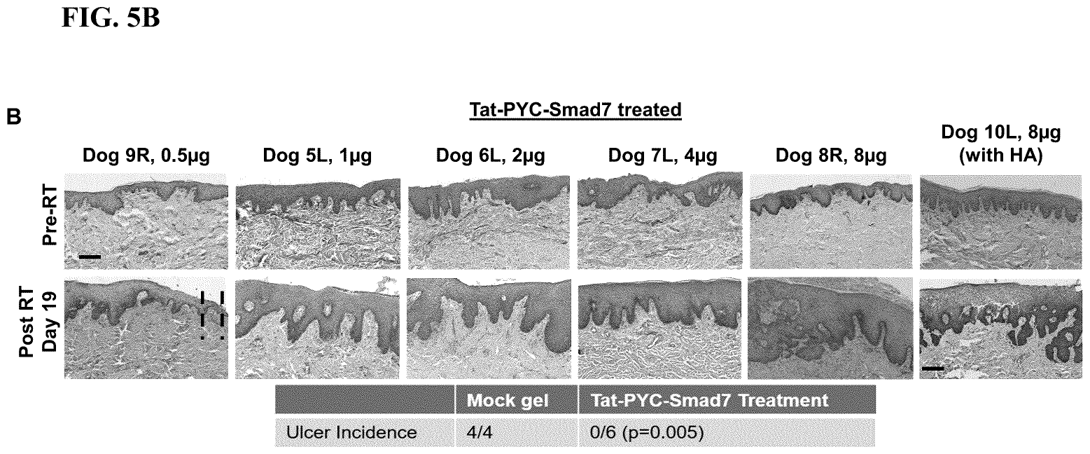

In another exemplary method, an IMRT dose of 20-30 Gy was delivered in 5 fractions of 4-6 Gy targeted to the mucosa in the first 4 beagles. The irradiated mucosa began to demonstrate erythema the second week after initial RT (FIGS. 4A and 4B) which is considered Grade 1 or Grade 0 oral mucositis, therefore a mock gel was applied daily from day 7 to day 19. The VRTOG scores demonstrated that the grades and duration of gross damage were dose-related with 6 Gy×5 most severe (FIGS. 4A and 4B). Histopathology of day 19 biopsies revealed that all three dosing protocols induced oral ulcers with 6 Gy×5 being the most severe in epithelial ablation and marked inflammation. Mandible mucosa had more epithelial ablation than maxillary mucosa in dog 3, whereas the left and right sides of maxillary mucosa in dog 4 were similar in VRTOG scores (FIG. 4B) and in histopathology severity in ulceration, inflammation and epithelial ablation (FIG. 5A). Clinical chemistry and hematology tests did not show consistent abnormalities, indicating the damage was largely restricted to local lesions.

Tat-PYC-Smad7 treatment. In another exemplary method herein, at weck 2 and weck 3 after initial RT, dogs were treated with HPC gel (vehicle, “mock gel”) on one lateral site (11.6 cm²) and Tat-PYC-Smad7 prepared as described in the example above (drug, 0.5-8 μg daily dose) on the adjacent lateral site (11.6 cm²) of irradiated area divided by ˜5 mm-10 mm dental mold; sites were randomly alternated for vehicle and drug among dogs. Each dog was under anesthesia during treatment, induced with midazolam (0.2 mg/kg) and propofol (3 mg/kg) and maintained on isoflurane gas (to effect), positioned in dorsal recumbency with the two sides separated by a divider mold, the dog was angled toward the treatment side during application, and the muzzle gently taped to ensure the topical treatment stayed within the irradiated field. Each side was exposed to 0.5 ml HPC gel with or without drug for 30 minutes under anesthesia, allowing natural draining out of the gel afterwards. Because it is difficult to determine the crossover amount of a drug to the adjacent vehicle side, the drug-treated site was used to evaluate pharmacodynamics (PD) markers. This set up allowed for double-blind treatment and histopathology evaluation. Dogs recovered from anesthesia without food/water intake for 1 hour after treatment.

The 5 Gy×5 IMRT regimen was used to test Tat-PYC-Smad7 oral gel treatment at the maxillary mucosa, starting day 8 after initial irradiation. Maxilla biopsies from dogs treated with mock oral gel only were used as vehicle control of histopathology and pharmacological dynamic markers, except dog #2 in which the epithelium was completely ablated. A “time-to-event continual reassessment method” of Phase I radiation trials in human patients was mimicked in this exemplary method for dose finding experiments. This design was adaptive and provided close estimates for maximal tolerated dose (MTD). The remaining 6 dogs were treated with Tat-PYC-Smad7 0.5-8 μg daily dose in 0.5 mL oral gel to cover the irradiated area. To confirm rapid drug-specific cell entry, an additional 8 μg dose of Tat-PYC-Smad7-HA protein was included (with the HA tag added to the C-terminal Tat-PYC-Smad7 protein), to allow HA-specific immunostaining.

Among dogs exposed to 5 Gy×5 RT in maxilla, although there are individual variations in VRTOG scores, compared to mock gel treated dogs (with 0.5 μg or without Tat-PYC-Smad7 treatment in neighboring mucosa), grade 3 scores were significantly shortened by Tat-PYC-Smad7 treatments, from 8.75±1.5 days to 4.16±2.3 days (FIGS. 4B and 4C). With the exception of 0.5 μg dose, in which the treated side had a much shorter grade 3 score than vehicle treated side (FIG. 4D), other treatment dogs had no ulceration in both Tat-PYC-Smad7 treated and neighboring sites (FIG. 4B), suggesting a bystander or systemic effect of Tat-PYC-Smad7 treatment with doses>0.5 μg/day. The dose-dependence of Tat-PYC-Smad7 was not obvious. Gross scores for all doses of Tat-PYC-Smad7 were better than vehicle treated in the 5 Gy×5 RT regimen.

Histopathology showed that all irradiated mucosa treated with mock gel only, and mock gel treated mucosa adjacent to 0.5 μg dose of Tat-PYC-Smad7 developed ulceration with marked inflammation (FIG. 5A, n=4). Tat-PYC-Smad7 treated mucosa post RT did not harbor ulceration, and inflammation is relatively mild (except one dog with 8 μg dose) in comparison with either vehicle treated mucosa or their own pre-RT biopsies normal mucosa (FIG. 5B, n=6, p=0.005 compared to vehicle, Fisher's exact). The lowest dose (0.5 μg) had a small area of epithelial thinning and erosion which was not seen in other doses. The 8 μg dose induced the most hyperproliferation compared to other doses with one showing profound inflammation.

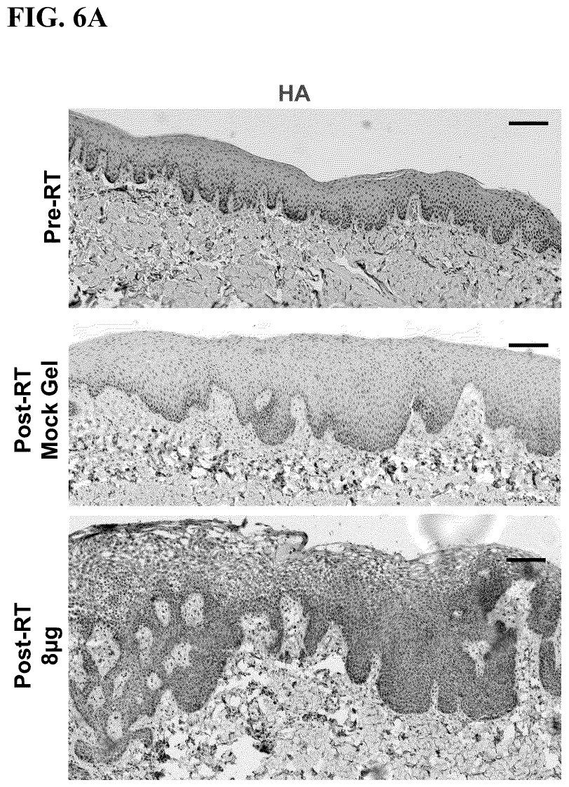

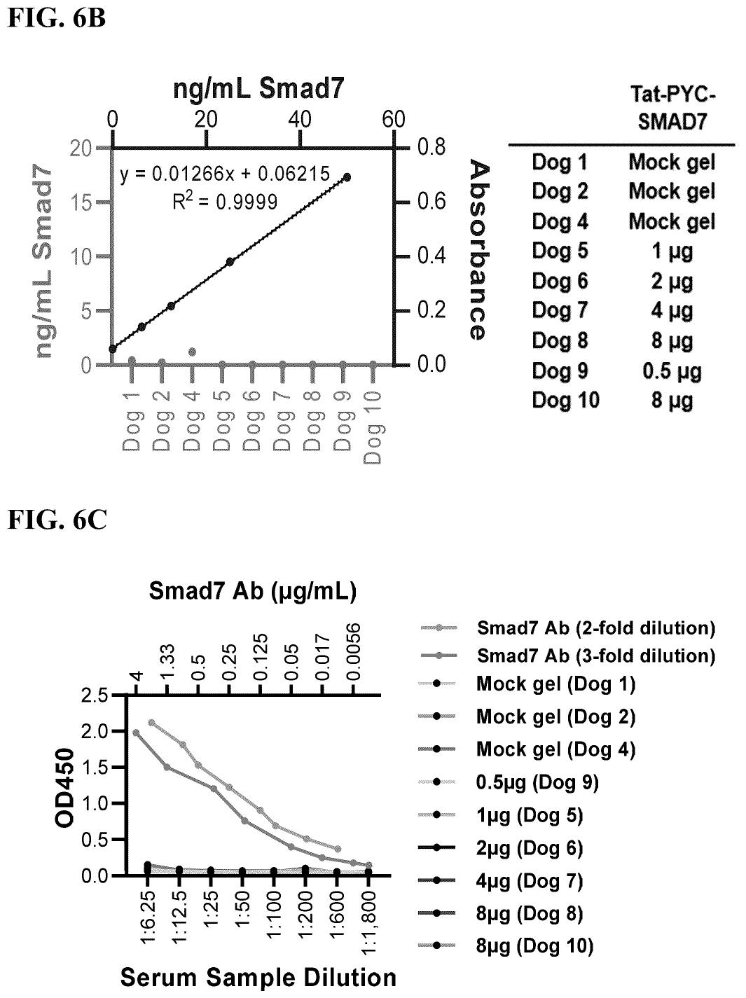

To confirm that Tat-PYC-Smad7 oral gel can rapidly enter damaged mucosa, biopsy tissue was treated with 8 μg daily dose of Tat-PYC-Smad7-HA to perform HA tag specific immunostaining. The final treatment was 30 minutes prior to biopsy. Tat-PYC-Smad7 was detected in treated mucosa primarily in epithelial cells, whereas the mock gel treated site did not show significant staining (FIG. 6A), indicating either no immediate crossover or it was too low to detect. Alternatively, systemic effects may be mediated by the changes in biomarkers (e.g., expression) such as those expressed and detected systemically.

Tat-PYC-Smad7 local application primarily targeted local tissue without eliciting anti-drug antibody (ADA) against itself. Because the mock gel treated site with neighboring Tat-PYC-Smad7 doses higher than 0.5 μg also demonstrated less severe pathological alterations compared to mock gel only treated animals, immunohistochemistry (IHC) was performed in this exemplary method to determine if Tat-PYC-Smad7 could crossover to the neighboring vehicle site. Tat-PYC-Smad7 was primarily identified at the treatment mucosal layers (FIG. 6A).

To determine if locally applied Tat-PYC-Smad7 entered the bloodstream thus causing systemic therapeutic effects on the neighboring vehicle site, a Smad7 ELISA assay was developed for use in the exemplary method herein. In brief, rabbit anti-Smad7 (Novus NBP1-87728) was coated onto 96-well plates overnight and served to capture Smad7. Mouse monoclonal antibody (R&D #MAB2029) that recognized the c-terminus of human SMAD7 was used for detection. HRP-conjugated horse anti-mouse-IgG (Cell Signaling Technologies) was used to detect captured immune complexes and 1-Step Ultra TMB-ELISA substrate (Thermo) was used to detect HRP activity followed by acid quenching and detection on a plate reader at 450 nm. Tat-PYC-Smad7 was spiked into untreated dog serum to establish a standard curve that detected Tat-PYC-Smad7 as low as 0.16 ng/mL. Smad7 levels in dog serum samples were measured before and after Tat-PYC-Smad7 treatment for 2 weeks, with the last treatment 30 minutes before serum was collected. Tat-PYC-Smad7 was not detected in the serum samples of dogs with any Tat-PYC-Smad7 doses (FIG. 6B). Therefore, the therapeutic effect of Tat-PYC-Smad7 was primarily local.

To determine if Tat-PYC-Smad7 elicits ADA that would neutralize its therapeutic effects, a Smad7 anti-drug antibody (ADA) ELISA was developed for use in the exemplary method herein. In brief, Tat-PYC-Smad7 was diluted in PBS to 20 ug/mL and coated onto 96-well plates at 100 μL/well and incubated overnight at 4° C. Plates were washed once with 0.05% Tween 20 in PBS (200 μl/well) followed by blocking for 2 hours at room temperature with 2% BSA in PBS (200 μL/well). Plates were again washed once with 0.05% Tween 20 in PBS (200 μl/well). Serum samples from mice treated with Tat-PYC-Smad7 and mock gel were first diluted 1:6.25 (the lowest possible dilution) followed by serial dilutions up to 1:800. A Smad7 mouse monoclonal antibody (R&D #MAB2029) was first diluted to 2 μg/mL followed by a ⅓ serial dilutions. Serum sample dilutions and Smad7 antibody dilutions were added to plates at 50 μL/well and incubated for 2 hours at room temperature. Plates were washed twice with 0.1% Tween 20 in PBS (200 μL/well). HRP-anti mouse IgG (Sigma #A4416) was diluted 1:20,000 in 2% BSA-PBS and added to plates at 100 μl/well for 1 hour at room temperature. Plates were washed 6 times with 200 μl/well of 0.1% Tween 20 in PBS. 1-Step Ultra TMB-ELISA (Thermo #34028) was added to plates at 50 μL/well and incubated at room temperature in the dark for 30 minutes. 50 μL/well of 2 M sulfuric acid was added to plates. A microplate reader (Biotek synergy 2) was used to measure optical density of each well at 450 nm, and OD 450 values were plotted in Graphpad Prism. As shown in FIG. 6C, none of the dogs with Tat-PYC-Smad7 doses showed ADA.

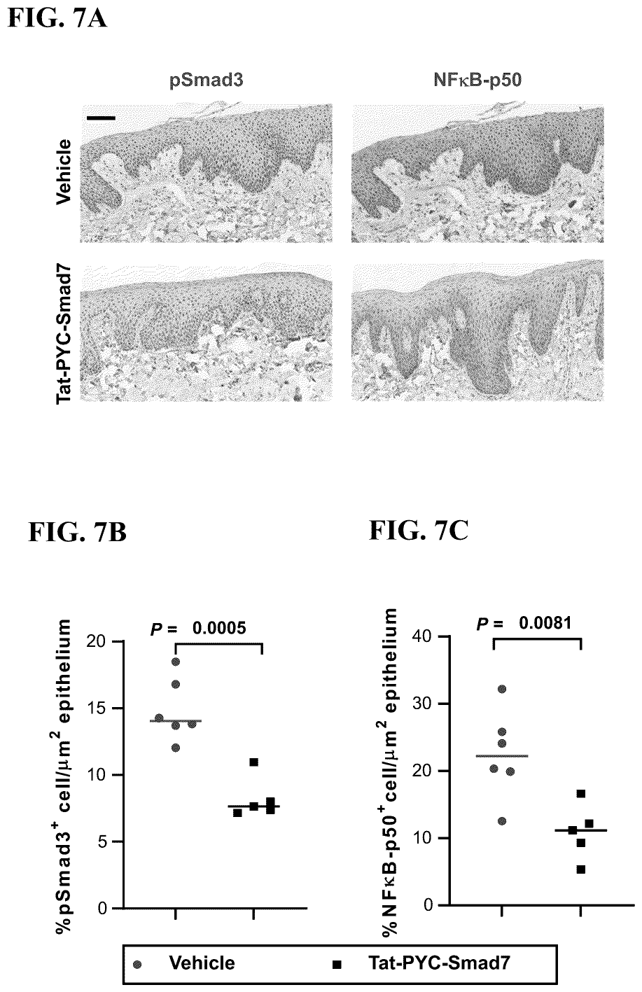

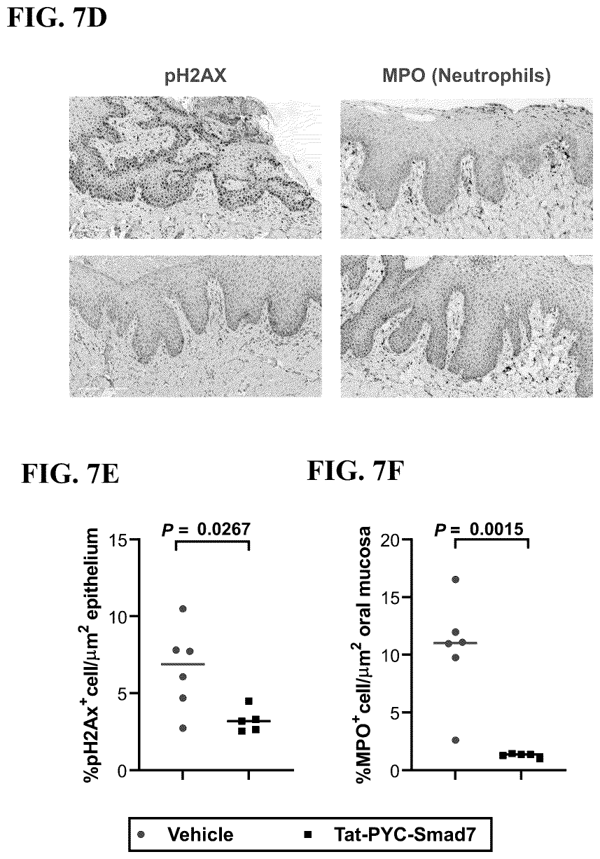

Consistent with previous data in mice showing a reduction in TNF/NFκB signaling, IHC performed in an exemplary method herein showed that nuclear NFκB-p50⁺ cells, a marker of NFKB pathway activation, were significantly reduced in oral mucositis lesions treated with Tat-PYC-Smad7 compared to mock gel treatment (FIGS. 7A and 7C). Further, pSmad3⁺ cells, a marker of TGF-β pathway activation, were reduced with Tat-PYC-Smad7 treatment (FIGS. 7A and 7B). MPO+ neutrophils were reduced by Tat-PYC-Smad7 treatment (FIGS. 7D and 7F) as were cells positive for pH2AX foci, a marker for unresolved DNA damage by RT (FIGS. 7D and 7E).

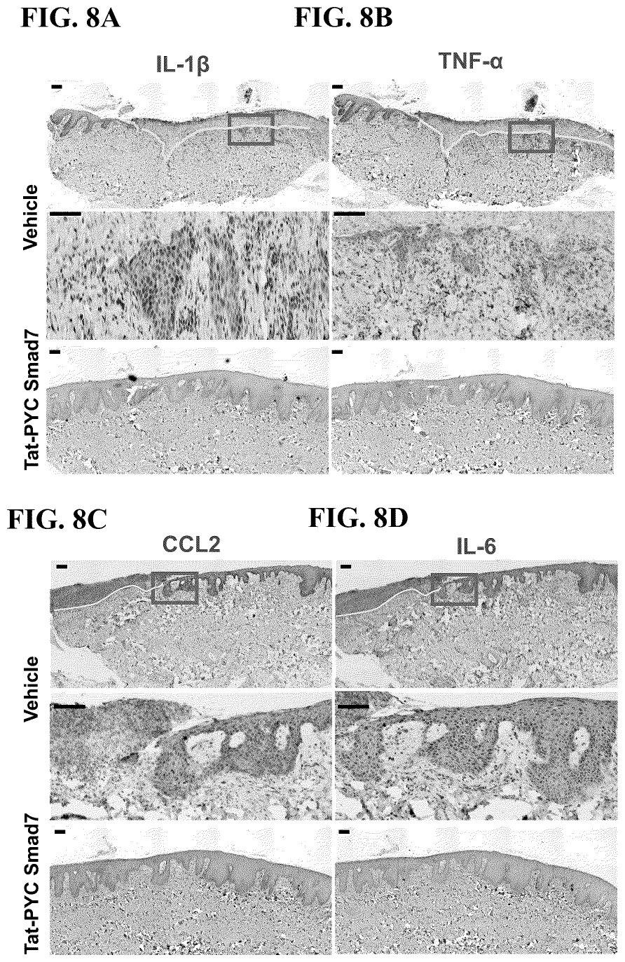

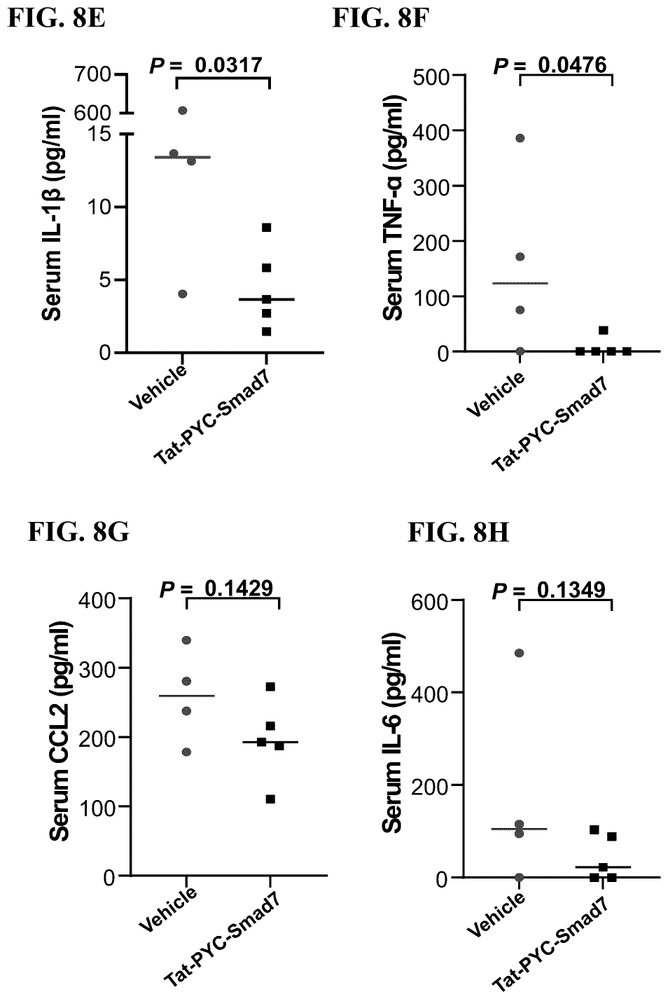

IHC was performed in an exemplary method herein to visualize spatial patterns in the lesions of inflammatory cytokines downstream of NFκB and/or TGF-β that were highly expressed in oral mucositis lesions. IL-1β (FIG. 8A), TNF-α (FIG. 8B), CCL2 (FIG. 8C) and IL-6 (FIG. 8D) were not detected in oral mucosa prior to RT, but these cytokines were detected in both oral epithelial cells and stromal cells after RT and were less prominent in Tat-PYC-Smad7 treated samples compared to vehicle.

To determine if these cytokines are secreted into the blood stream, a Cytokine Luminex assay was performed in this exemplary method. In brief, MILLIPLEX Canine Cytokine/Chemokine Magnetic Bead Panel-Immunology Multiplex Assay (MilliporeSigma CCYTMG-90K-PX13), which detects TNF-α, IL-6, CCL2, IL-8 and IL-10 was used to detect serum levels of these cytokines. The assay used the Luminex xMAP platform which employs a magnetic bead immunoassay format for fluorometric detection of the cytokines in a sample. 50 μL serum was used. Separately, serum levels of IL-1β were detected using an ELISA (Duoset for canines, R&D #DY3747) according to the manufacturer's protocol. 100 μL serum/dog was used for detection. Of the cytokines measured herein, TNF-α (FIG. 8F) and IL-1β (FIG. 8E) were significantly reduced, and IL-6 (FIG. 8H) and CCL2 (FIG. 8G) were trending lower in serum samples of dogs treated with local Tat-PYC-Smad7 treatment in comparison with serum samples of dogs treated with mock gel. Together, these data suggest treatment with Tat-PYC-Smad7 surprisingly results in not just local; but also systemic reductions of inflammatory cytokines/chemokines in irradiated dogs despite having no detectable levels of Tat-PYC-Smad7 in the blood (FIG. 6B).

In the exemplary methods provided herein in this example, the topical Tat-PYC-Smad7 application demonstrated therapeutic effects on IMRT-induced oral mucositis in dogs and the local effects of Tat-PYC-Smad7 on targeting molecules were involved in oral mucositis pathogenesis as well as systemic reductions in inflammatory cytokines.

### Example 3

In another exemplary method, a recombinant Tat-tagged protein containing amino acids 203-426 of human Smad7 (Tat-PYC-Smad7) with a human influenza hemagglutinin (HA) tag was produced using methods similar to those described in the example above. Tat-PYC-Smad7-HA was mixed with a HPC-base gel or HEC-base gel prepared as described in the example above (e.g., see Table 3). Specifically, 0.25 μg, 0.5 μg, or 1 μg Tat-PYC-Smad7-HA was mixed 1:1 with 30 μl of the HPC-base gel resulting in a formulation having a final concentration of 0.125 μg, 0.25 μg, or 0.5 μg Tat-PYC-Smad7-HA.

In one exemplary method, the Tat-PYC-Smad7-HA gel formulations were used to topically treat an imiquimod-induced skin inflammation in a mouse model of psoriasis or contact dermatitis/atopic dermatitis depending on the mouse strain tested. Psoriasis is a chronic skin condition associated with multiple contributing factors including autoimmune disease. The imiquimod-induced psoriasis model is particularly translational into the clinic as it has many of the significant markers of human disease, including histopathology of lesions and strong activation of the immune system. Imiquimod (IMQ) is a topical anti-tumor medication that can treat genital warts as well as rough, raised areas of heavily sun-exposed skin (actinic keratoses) and skin cancer (basal cell carcinoma). In brief, eight-week-old C57BL/6J-Tyr<c-2J>/J female (albino) mice (5 mice/group) were used in the exemplary method herein. Mouse back skin was shaved followed by topical application of 62.5 mg IMQ cream daily (days 1 to 4) to develop inflammation. The stratum corneum was compromised by Day 2 of IMQ treatment. Tat-PYC-Smad7-HA was combined with HC gel to form drug product. Starting on Day 2, Tat-PYC-Smad7-HA gel was applied at a dose of 0.125 μg/day, 0.25 μg/day or 0.5 μg/day after IMQ application, daily through day 6. Mock HPC gel (i.e., the HPC-base gel with protein dilution buffer in place of Tat-PYC-Smad7-HA) was used as vehicle control. Gross appearance of skin inflammation was monitored daily, and skin was excised on day 6 for histology analysis.

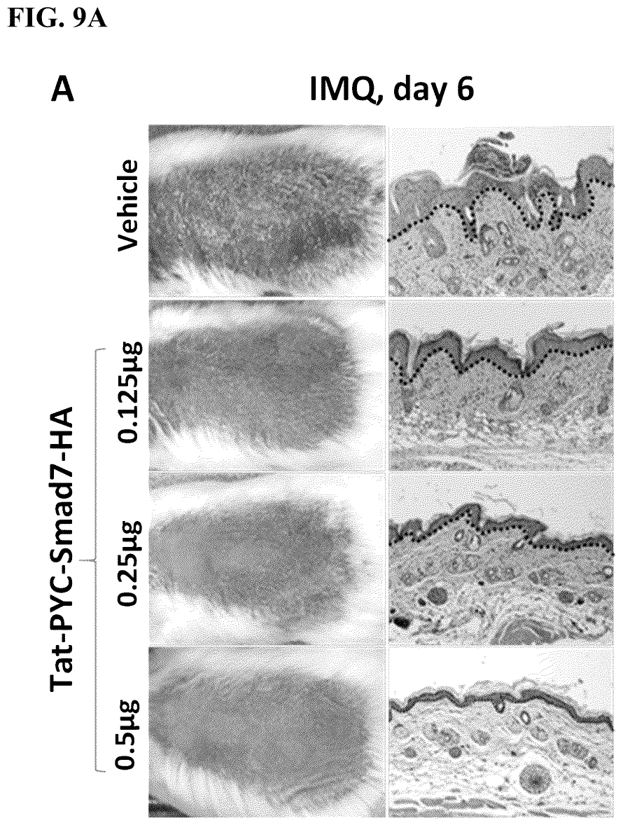

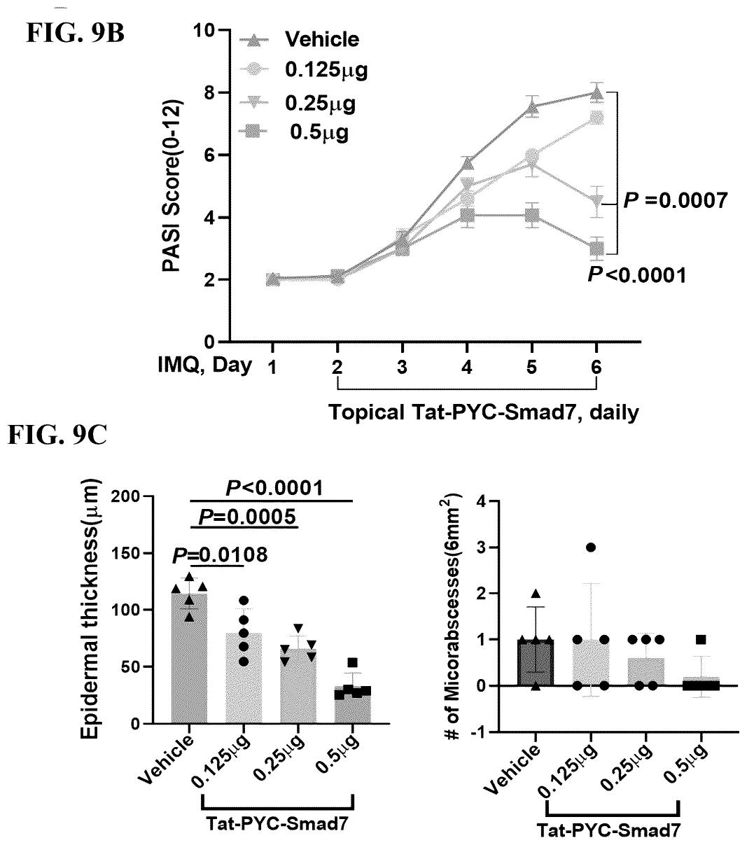

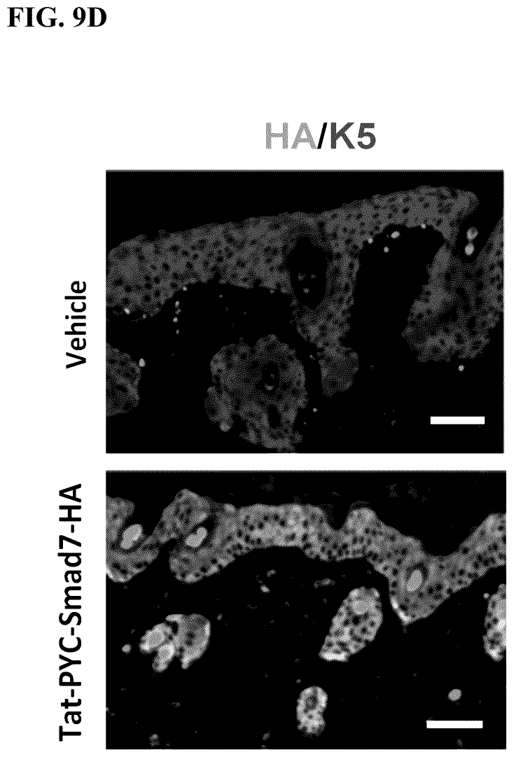

Results, as presented in FIGS. 9A-9D, show that Tat-PYC-Smad7 treated mouse skin had a gross reduction in scaly patches and erythema (FIG. 9A) and a dose-dependent reduction in the psoriasis score (PASI) used in clinic (FIG. 9B). At the histopathological level, Tat-PYC-Smad7-HA treated skin dose-dependently showed reduced epidermal hyperplasia and inflammatory cells compared to vehicle control (FIGS. 9A and 9C). Immunofluorescence staining of the HA tag of Tat-PYC-Smad7-HA showed that the protein penetrated into the epidermis and dermis (FIG. 9D). Additional experiments performed on FVB and Balb/c mice showed similar results (data not shown but available on request).

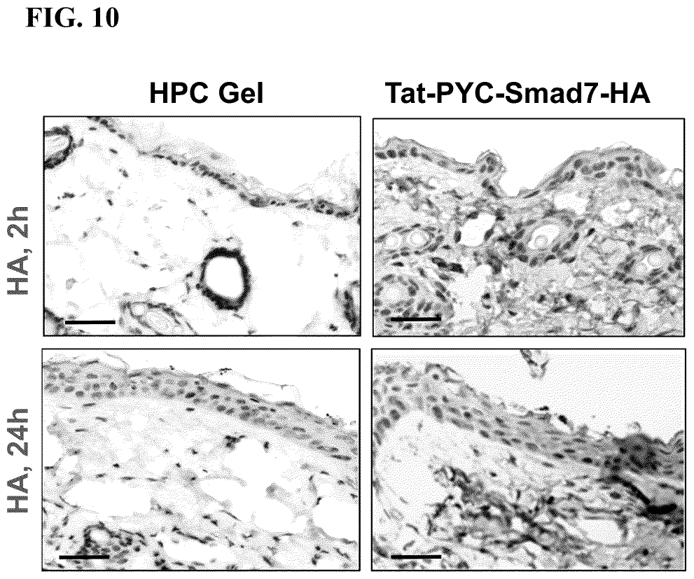

In one exemplary method, 3 μg of Tat-PYC-Smad7-HA in 50 μl HPC gel formulation were used to topically treat each of 4 mm-excisional wounds in mouse skin to assess cell entrance of Tat-PYC-Smad7 in animals. Skin adjacent to the wounds was excised for HA IHC 2 hours or 24 hours afterwards. Tat-PYC-SMad7-HA was detected in sections from 2 hours and 24 hours demonstrating protein transduction and persistence at the site of the wound (FIG. 10). As shown in FIG. 10, adjacent skin was excised at two and 24 hours following topical application of Tat-PYC-Smad7 in HPC gel. Antibody against c-terminal HA tag was used for immunostaining and the scale bar as shown is 50 μm. At 2 hours, epidermis adjacent to the wound showed more hyperplasia than immediately after wounding.

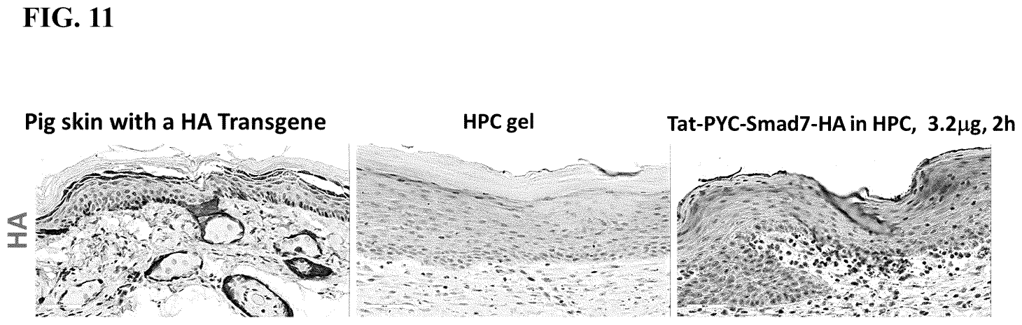

In one exemplary method, 3.2 μg of Tat-PYC-Smad7-HA in 0.5 ml HPC-base gel topically applied to the pig skin from newly healed 1.5 cm-excisional wound represents exemplary experiments of the instant disclosure, illustrating the penetration of HPC formulated, topically applied, Tat-PYC-Smad7-HA in newly healed pig skin (FIG. 11). 3.2 μg protein was topically applied to newly healed skin from 15 mm excisional wound, and skin was excised for immunostaining of the HA tag in Tat-PYC-Smad7 2 hours afterwards. Pig skin expressing a HA transgene in basal layer of the epidermis is used as a positive control. Antibody against c-terminal HA tag was used for immunostaining.

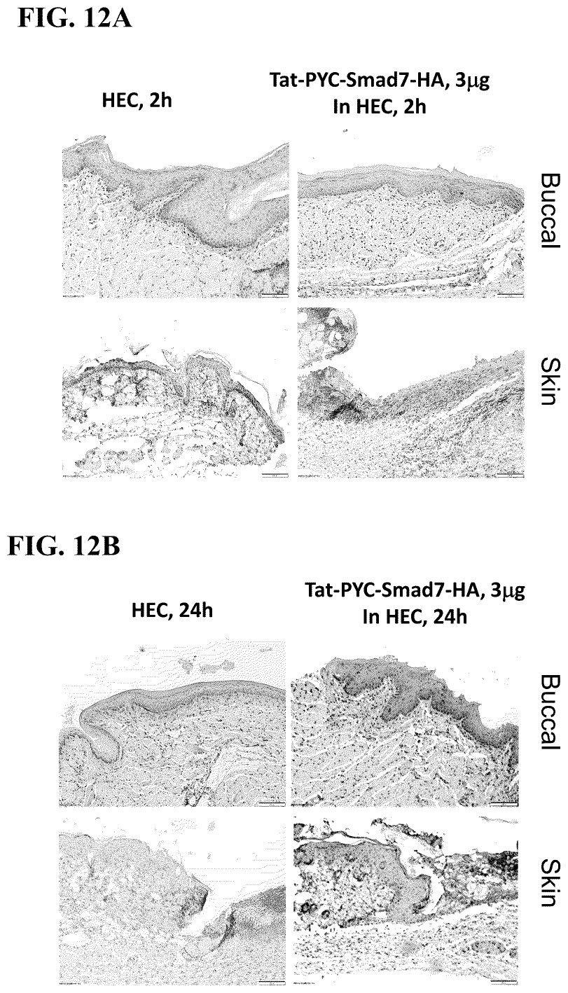

In one exemplary method, 3 μg of Tat-PYC-Smad7-HA in HEC-base gel topically applied to the oral cavity or 4 mm excisional wound skin of mice, showed similar results to application using Tat-PYC-Smad7 in HPC-base gel following histological staining of punch biopsies taken 2 hours (FIG. 12A) and 24 hours (FIG. 12B) after application of Tat-PYC-Smad7 in HEC-base gel. Tat-PYC-Smad7-HA was primarily present in the epithelial layers of the buccal cavity and wound bed in skin at 2 hours and remained present at least through 24 hours.

### Example 4

In another exemplary method, a recombinant Tat-tagged protein containing amino acids 203-426 of human Smad7 (Tat-PYC-Smad7) was produced using methods similar to those described in the examples above. Tat-PYC-Smad7 was mixed with a HPC-base gel prepared as described in the example above (e.g., see Table 3). Specifically, 0.5 μg Tat-PYC-Smad7 was mixed 1:1 with 30 μl of the HPC-base gel resulting in a formulation having a final concentration of 0.25 μg Tat-PYC-Smad7 to treat radiodermatitis in mice.

In one exemplary method, the Tat-PYC-Smad7-HA gel formulations were used to topically treat radiodermatitis skin inflammation in a mouse model. C57BL/6J-Tyr<c-2J>/J female, 8-week old mice (JAX) were exposed to radiation using smART, a small animal irradiator mimicking SBRT in clinic, to simultaneously irradiate the flanks of both hind legs (40 Gy, 5.2 Gy/min dose rate). Irradiation of flanks avoids gut toxicity by residual RT penetration. One of the irradiated legs was treated with HPC alone as a control, and the other with HPC gel formulated Tat-PYC-Smad7. Dermatitis severity was scored with a modified version of the NCI's criteria (Table 6), Gross Radiodermatitis score (0-4, modified based on RTOG and NIH CTCAE criteria).

TABLE 6

| Score | Description |
|---|---|
| 0.5 | Faint erythema, epilation < 25% area |
| 1 | Faint to moderate erythema, epilation 25%-50% |
| 1.5 | Moderate erythema, epilation > 75% |
| 2 | Erythema with dry desquamation < 25% area; mild edema |
| 2.5 | Moist desquamation in more than one small area, or |
| | erythema with branny/scaly desquamation in > 50% area |
| 3 | Moist desquamation in < 25% area, or peeling in sheets; |
| 3.5 | Moist desquamation > 50% area or patchy crusting |
| 4 | Ulceration, hemorrhage, necrosis |

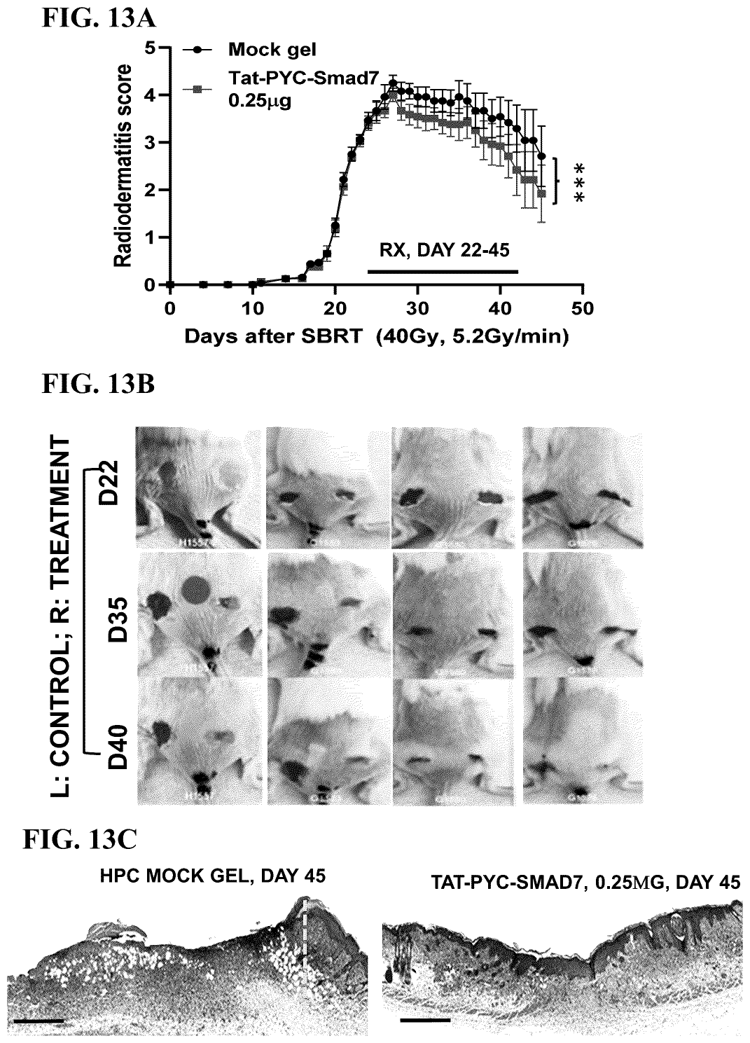

Both irradiated flanks developed radiodermatitis with similar kinetics and severity. Treatment using a daily 0.25 μg dose of HPC-formulated Tat-PYC-Smad7 (ulcer size is one fourth of a diabetic wound, so the dose was reduced accordingly) was initiated on one lesion when dermatitis on both sides scored 3. The dose is the equivalent to 5 μg in dog for dose/cm.² A reduction in radiodermatitis severity was observed (FIGS. 13A-13C).

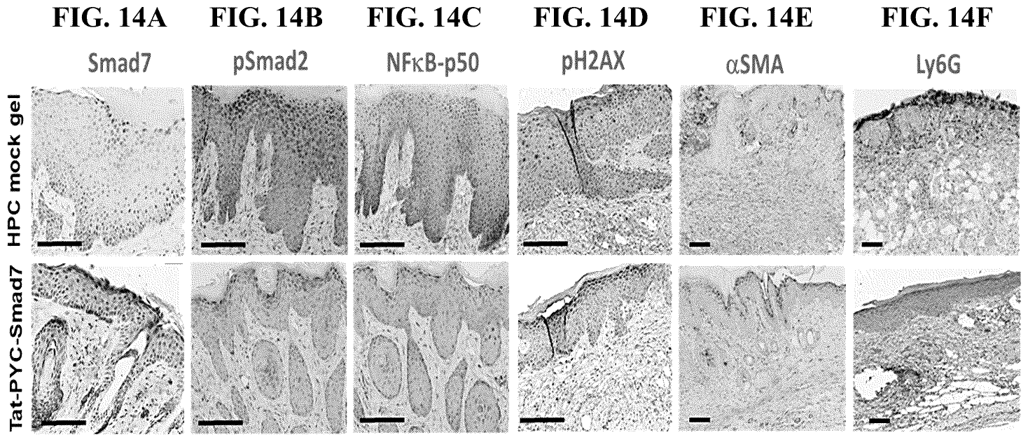

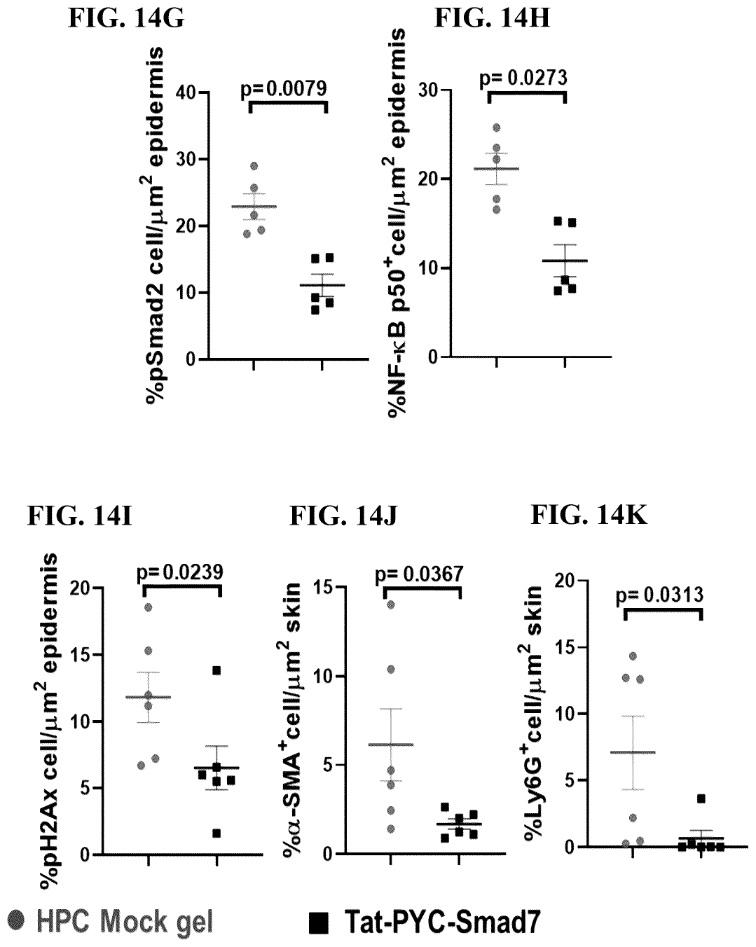

In mouse radiodermatitis, IHC using a c-terminal specific Smad7 antibody (R&D Systems) reveals that endogenous Smad7, a TGFβ transcriptional target, was elevated primarily in basal keratinocytes of hyperplastic epidermis adjacent to ulcers. In Tat-PYC-Smad7 treated lesions, Smad7 (both endogenous and exogenous) staining is throughout all layers of the epidermis (FIG. 14A). PD marker analyses (pSmad2, FIGS. 14B and 14G; NFkB-p50, FIGS. 14C and 14H; pH2AX, FIGS. 14D and 14I) showed effects of Tat-PYC-Smad7 similar to those in radiodermatitis on dog skin. Additionally, the fibrotic response, marked with α-smooth muscle actin (αSMA)⁺ fibroblasts (FIGS. 14E and 14J), and unresolved inflammation, marked with Ly6G⁺ neutrophils (FIGS. 14F and 14K), became more obvious than in dog lesions, and was attenuated by Tat-PYC-Smad7 treatment. Each data point shown in FIGS. 14G-14K was averaged from 3 IHC sections; quantification from paired samples is presented with Mean±SEM and analyzed by student T-test. Scale bar: 50 μm.

### Example 5

In another exemplary method, a recombinant Tat-tagged protein containing amino acids 203-426 of human Smad7 (Tat-PYC-Smad7-HA) was produced using methods similar to those described in the example above. Tat-PYC-Smad7-HA was mixed with a HPC-base gel prepared as described in the example above (e.g., see Table 3). Specifically, 2.4 μg or 9.6 μg Tat-PYC-Smad7-HA was mixed 1:2 with of the HPC-base gel resulting in a formulation having a final concentration of 0.8 μg, 3.2 μg Tat-PYC-Smad7-HA in 0.5 ml to cover 1.5 cm diameter surgical wound in pig skin.

In one exemplary method, the Tat-PYC-Smad7-HA gel formulations were used to topically treat wounds in a pig model of wound healing. Pig skin closely mimics human skin. Because Tat protein cannot penetrate intact stratum corneum, a pilot experiment was performed at Bridge PTS, a CRO company, in which Tat-PYC-Smad7 in HPC was applied to excisional wounds of a pig. One specific pathogen-free, female, commercially raised, Yorkshire-cross pig (32 Kg), that has normal wound healing at this age, was purchased and delivered to the testing facility eight days prior to surgery. Twenty-four, full-thickness, 1.5 cm diameter wounds were created on the pig in a precise pattern to allow for assessment of systemic exposure (data not shown but available on request). Wounds in different locations were divided in 3 groups treated as follows: 1) vehicle (HPC, n=8); 2) drug, dose 1 (0.8 μg/wound, n=8); 3) drug dose 2 (3.2 μg/wound, n=8). Treatments occurred on Day 0, 2, 5, 7, 9, 12, and 14 (each dressing change except on Day 14). Test/control article were applied directly onto the wound to ensure full coverage. Test/control were exposed to air for 30-60 minutes following application (i.e. not covered with a dressing during this time). An occlusive dressing was applied over each wound prior to bandaging. Blood/Urine collection was on Days 0 and 14 and Sample was on Day 14.

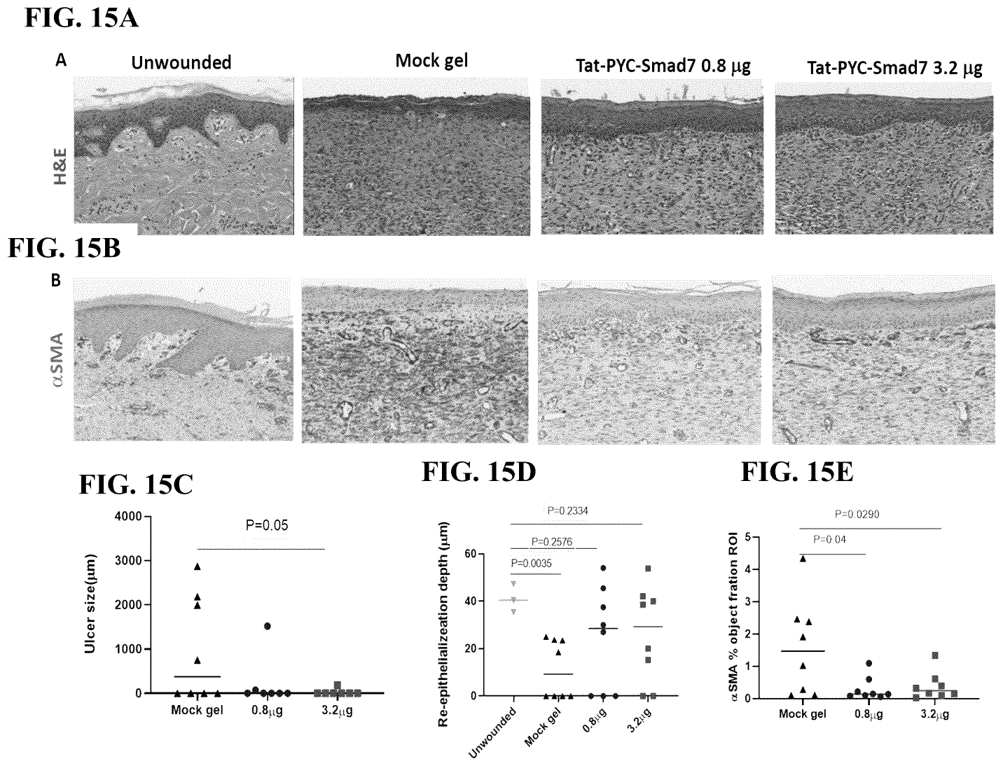

Tat-PYC-Smad7 gel penetrated to the wound epidermis and stromal cells (FIGS. 15A-15E). In this young healthy pig, wound healing was typically rapid, thus further accelerated wound closure was not observed by Tat-PYC-Smad7 treatment (Data not shown but available on request). For all wounds, no dose-dependent erythema or edema were observed, and all wound granulated normally. Clinical chemistry for the blood and urine and hematology on day 14 were all normal and did not differ from day 0. Therefore, no cutaneous or systemic toxicity was identified by topical application of the indicated dose of Tat-PYC-Smad7 and by the formulation excipients. Microscopically, Tat-PYC-Smad7 gel treated wounds had fewer remaining ulcers than mock gel treated, and thicknesses of newly re-epithelialized epidermis were more similar to unwounded epidermis. Additionally, αSMA, a fibrogenic marker abundant in the wound stroma, was significantly reduced by Tat-PYC-Smad7 treatment,), indicating better wound remodeling and suggesting the Tat-PYC-Smad7 application can also reduce the risk of scar formation. In another example, Tat-PYC-Smad7 treated skin demonstrated reduced nuclear pSmad2 and NFkB, the two reliable PD markers (indicators of Smad7 activity) in treated wounds (data not shown but available on request).

FIGS. 15A-15E illustrates better re-epithelialization of Tat-PYC-Smad7-HA treated pig wounds. FIG. 15A shows representative histology of re-epithelialized skin wounds. FIG. 15B shows immunostaining of αSMA showing treated wounds were closer to normal skin, quantification is provided in FIG. 15E. FIG. 15C shows the number of microscopic ulcers in each group. FIG. 15D shows the thickness of re-epithelialized epidermis show treated groups were similar to unwounded skin whereas mock gel treated were significantly thinner.

### Example 6

In one exemplary method, the Tat-PYC-Smad7 gel formulations were used to topically treat mouse models of skin inflammation.

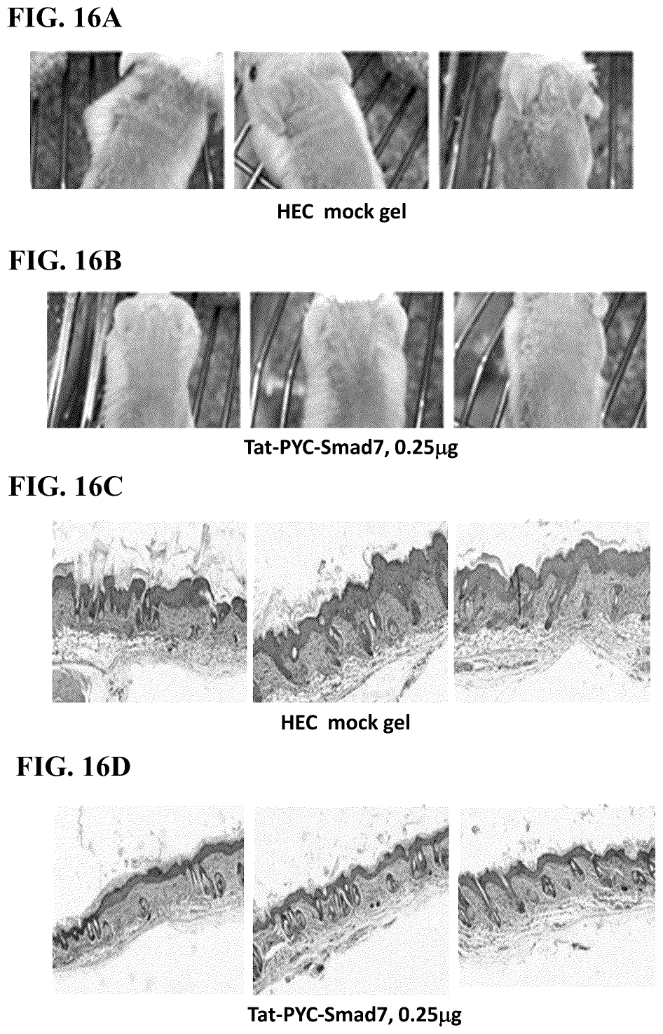

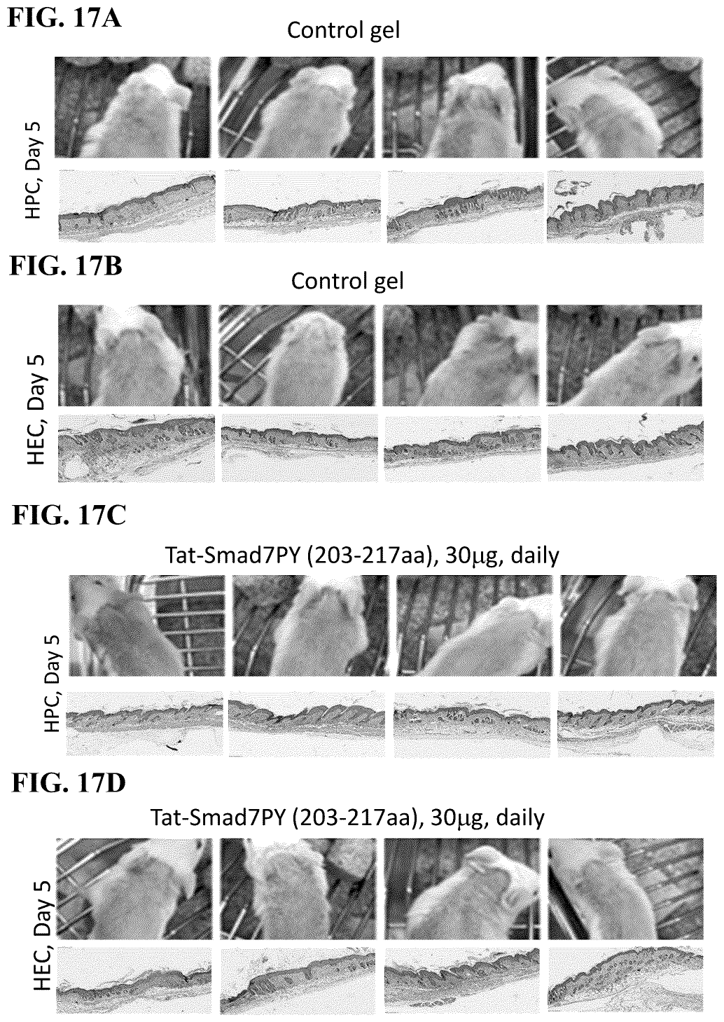

In one exemplary method of skin inflammation induced by IMQ, eight weeks old female JAX #000058 C57BL/6J-Tyr/J (JAX) mice were used. IMQ 62.5 mg cream was applied daily from day 1 to day 5, and Tat-Smad7 peptide in HPC or HEC was applied from day 2 to day 5, 4 hours after IMQ application on overlapping days. In mice treated with 0.25 μg or 0.5 μg Tat-PYC-Smad7 in HEC gel, gross photos showed thickened erythema skin in HEC mock gel treated mice, but less so in Tat-PYC-Smad7 treated mice (FIGS. 16A-16B). FIGS. 16C-16D show the corresponding histology depicting significant epidermal hyperplasia and infiltrated immune cells in the dermis of the mock gel group, and much less epidermal hyperplasia and infiltration in Tat-PYC-Smad7 treated skin. In mice treated with 30 μg of Tat-PY-Smad7 in HPC or HEC, gross photos showing thickened erythema skin in HPC or HEC mock gel treated mice, but less so in Tat-PY-Smad7 treated mice (FIGS. 17A-17D). Corresponding histology under each gross photo showed significant epidermal hyperplasia and infiltrated immune cells in the dermis in the mock gel group, which was moderately reduced in Tat-Smad7PY treated skin (FIGS. 17A-17D).

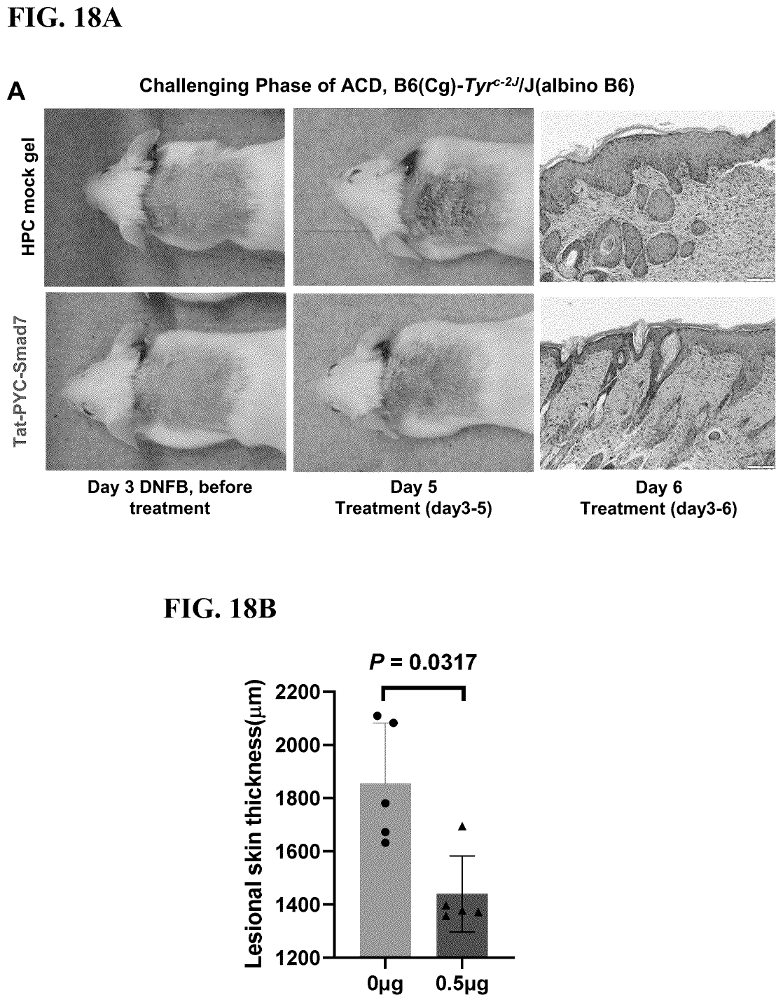

In one exemplary method, skin inflammation induced by DMFB was used as a model of contact dermatitis in mice. DNFB (2,4-dinitrofluorobenzene) treatment: 50 μl of 0.2% DNFB in acetone/sesame oil (4:1) was applied on dorsal skin on day 1, 3 and 5. In FIG. 18A, gross appearance and histology of skin inflammation and epidermal thickness was reduced by Tat-PYC-Smad7 (0.5 μg in HPC gel, daily, day 3-6). In FIG. 18B, quantification of skin thickness in FIG. 18A determined by microcaliber is shown. N=5 mice/group.

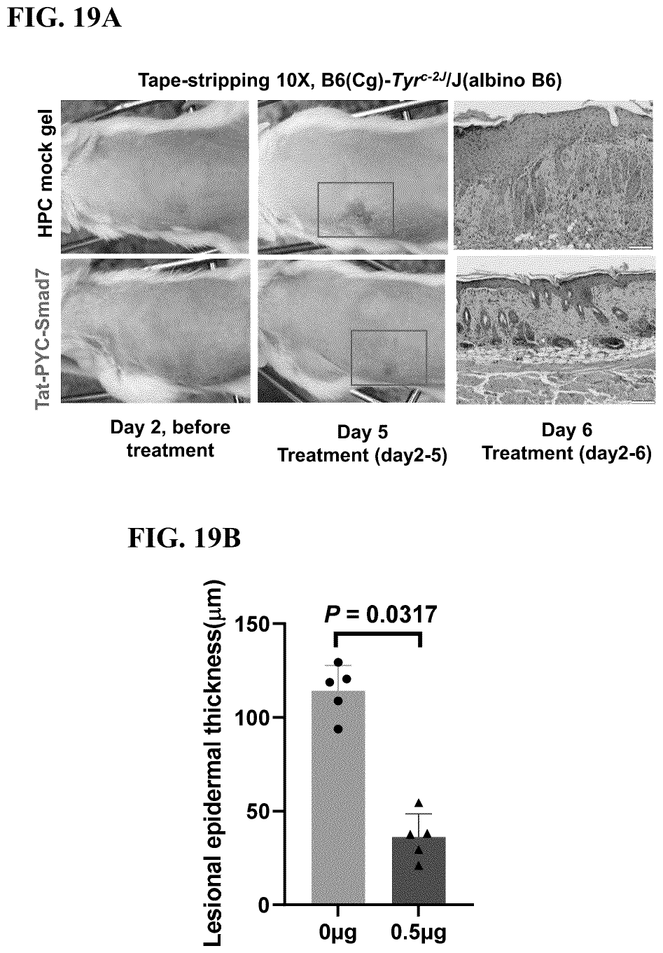

In one exemplary method, skin inflammation induced by tape stripping of hairless mouse skin served as a model for barrier defect atopic dermatitis. Scotch tape stripping (10 times) was introduced on hairless skin on day 1. FIG. 19A shows that gross appearance and histology of skin inflammation was reduced by treatment with Tat-PYC-Smad7 (0.5 μg in HPC gel, daily, day 3-6). Tissue within the squares shown in FIG. 19A depict the skin that had the most severe inflammation in each mouse and reflects the skin tissue that was collected and processed for histology. FIG. 19B shows quantification of epidermal thickness by H&E staining of histology sections of the tissue shown in FIG. 19A. Tat-PYC-Smad7 treatment also reduced inflammatory cells in the dermis compared to HPC mock gel. N=5 mice per group.

### Example 7

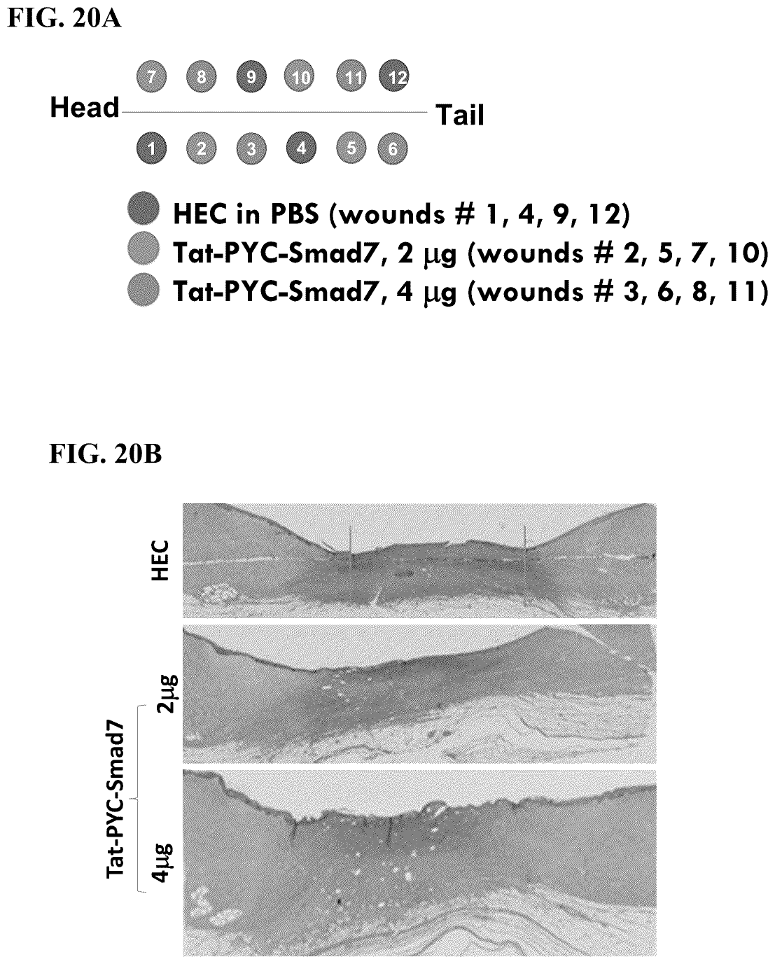

In another exemplary method, Tat-PYC-Smad7 gel formulations were used to topically treat pig models of diabetic pig wounds. A diabetic pig model was used in these methods and is excepted as a representative model for diabetic wounds in other settings such as humans. In this example, castrated Yucatan minipigs, 6-7 Months of age, were subjected to chemically induced diabetes. Once diabetes was established in the pigs, twelve, full-thickness, 2 cm diameter wounds were created on dorsal skin of the pig. Tat-PYC-Smad7 was formulated into a gel formulation at two dosages (2 μg or 4 μg) in a base gel that contained 1% HEC, 30% glycerol in PBS, by mixing in a 1:1 ratio Tat-PYC-Smad7 in PBS with the base gel (final 0.5% HEC, 15% glycerol, 0.0025% tween-80). As illustrated in FIG. 20A, Tat-PYC-Smad7 was topically applied to the wounds via the gel formulations in different wound locations. Wounds were treated with either 1) vehicle; 2) 2 μg Tat-PYC-Smad7/wound; or 3) 4 μg Tat-PYC-Smad7/wound where vehicle refers to the base gel formulation mixed at a 1:1 ratio with PBS (FIG. 20A). Treatments occurred on Day 2, 5, 7, and 10. Gel formulations were applied (0.25 ml) directly onto the wound to ensure full coverage. An occlusive dressing was applied over each wound prior to bandaging. Final treatment was on day 16, followed by euthanasia of the pig 1 hour later. Skin surrounding each wound was then taken for histology as frozen tissue samples. Frozen tissues were later sectioned and subjected to H&E staining. FIG. 20C illustrates representative histopathology images in the wounded area (day 16). The vertical lines in the image of vehicle-treated skin indicate the remaining open wound (FIG. 20C, top panel), whereas the Tat-PYC-Smad7-treated wounds show evidence of epidermal migration thus closing the wound (FIG. 20C, middle and bottom panels). A summary of the accelerated wound closure (as evaluated by histopathology evaluation) by Tat-PYC-Smad7 treatment on day 16 is provided in Table 7.

TABLE 7

| | | Tat-PYC-Smad7 | Tat-PYC-Smad7 |
|---|---|---|---|
| Group | HEC | (2 μg) | (4 μg) |
| Wounds completed, closure | 1 | 4 | 4 |
| Open wounds | 3 | 0 | 0 |
| P value to HEC | — | 0.03 | 0.03 |

### Example 8

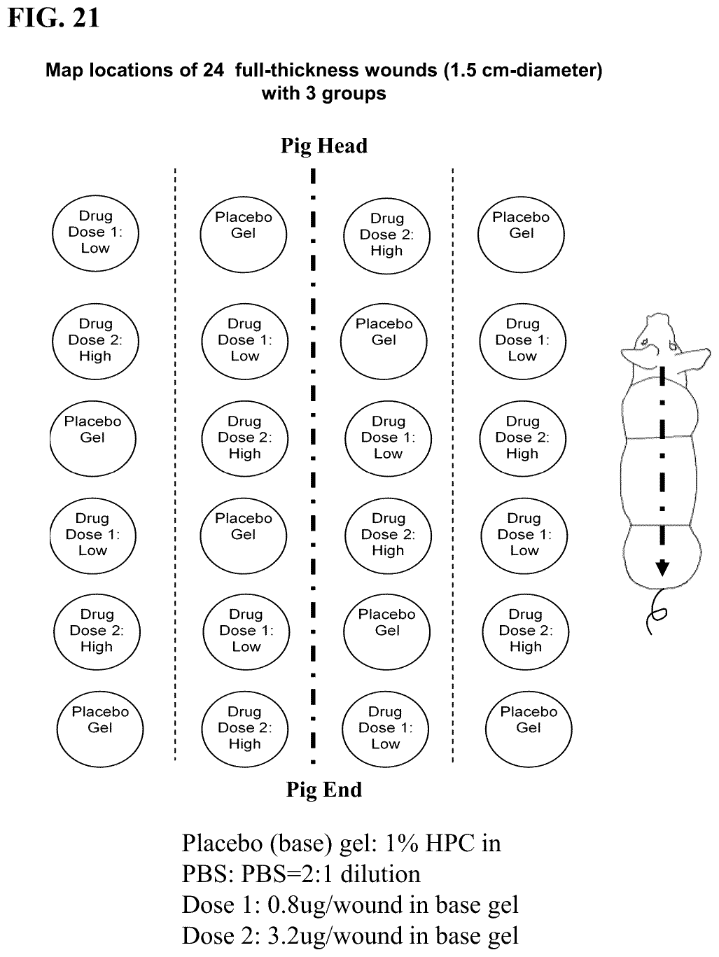

In one exemplary method, the Tat-PYC-Smad7 gel formulations were used to topically treat excisional wounds of healthy pigs. The pig used in the method was a pathogen-free, female, commercially raised, Yorkshire-cross pig (32 kg). The pig had normal wound healing capabilities and delivered to the testing facility eight days prior to surgery. Twenty-four, full-thickness, 1.5 cm diameter wounds were created on the dorsal skin of the pig. Tat-PYC-Smad7 was formulated into a gel formulation at two dosages (0.8 μg (“low dose”) or 3.2 μg (“high dose”)) in a base gel that contained 1% HPC in PBS, by mixing in a 2:1 ratio Tat-PYC-Smad7 in PBS with the base gel. As illustrated in FIG. 21, Tat-PYC-Smad7 was topically applied to the wounds via the gel formulations in different wound locations. Wounds were treated with either 1) vehicle; 2) low dose Tat-PYC-Smad7 (0.8 μg/wound; or 3) high dose Tat-PYC-Smad7 (3.2 μg/wound) where vehicle refers to the base gel formulation mixed at a 2:1 ratio with PBS (FIG. 21). Treatments occurred on Day 0, 2, 5, 7, 9, 12, and 14. Urine and blood were collected on each day of treatment and subjected to standard blood chemistries and urinalysis. Each treatment day, the dressing was changed except on Day 14 (D14). The gel formulations were applied directly onto the wound to ensure full coverage. The gel formulations were exposed to air for 30-60 minutes following application (i.e., not covered with a dressing during this time). An occlusive dressing was applied over each wound prior to bandaging. After final treatment the pig was euthanized and skin surrounding each wound was harvested for histology and processed as frozen tissue samples. Frozen tissues were later sectioned and subjected to H&E and IHC staining.

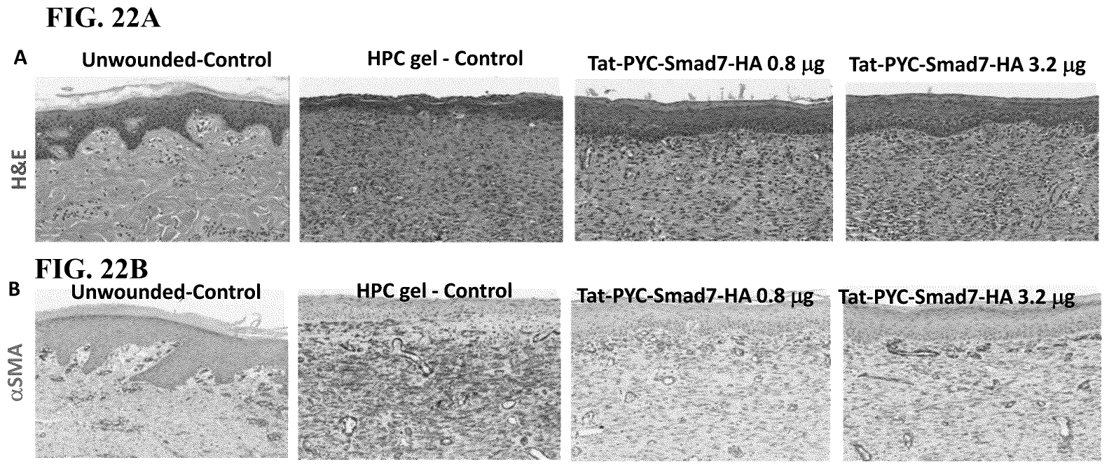

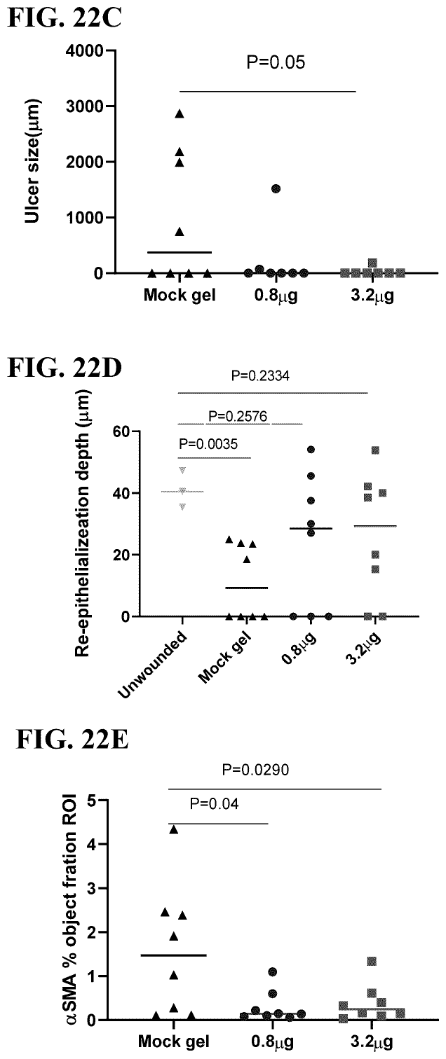

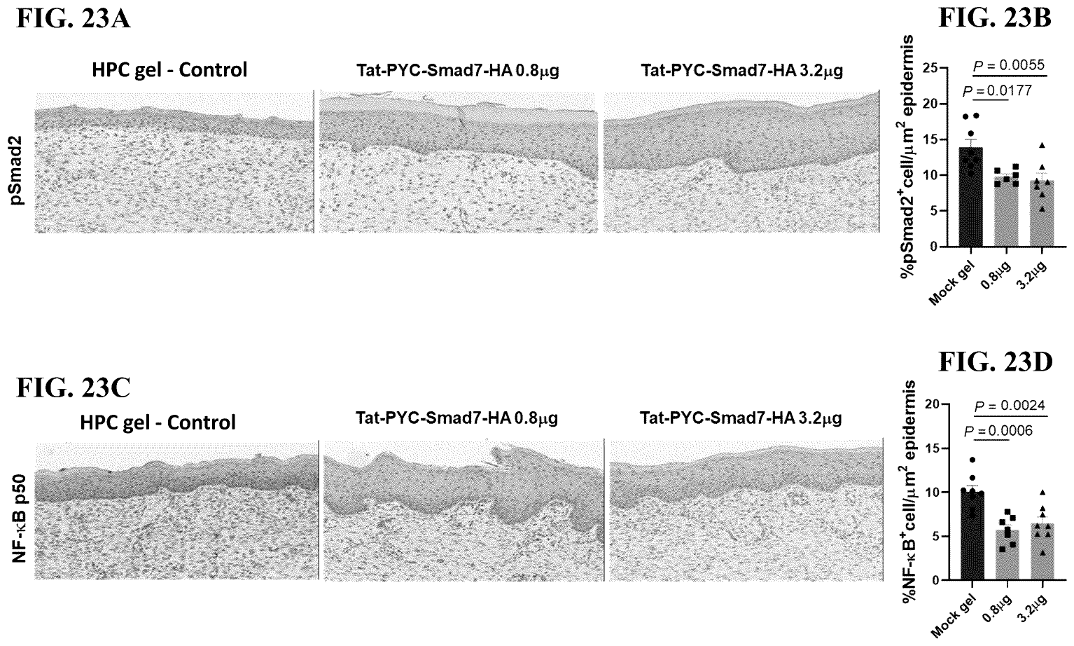

For all wounds, no dose-dependent erythema or edema were observed, and all wound granulated normally. Clinical chemistry for the blood and urine and hematology on day 14 were all normal and not differ from day 0. Therefore, no cutaneous or systemic toxicity was identified by topical application of the indicated dose of Tat-PYC-Smad7 and by the formulation expedients. Microscopically, Tat-PYC-Smad7 gel treated wounds had fewer remaining ulcers than mock gel treated (control) (FIGS. 22A and 22C), and the thickness of re-epithelialized epidermis in Tat-PYC-Smad7 treated groups were similar to unwounded or control skin whereas HPC gel treated wounds had significantly thinner epidermis (FIGS. 22A and 22D). Additionally, αSMA, a fibrogenic marker abundant in the wound stroma, was significantly reduced by Tat-PYC-Smad7 treatment, indicating a better wound remodeling (FIGS. 22B and 22E). In addition, Tat-PYC-Smad7 treated skin demonstrated reduced nuclear pSmad2 (FIGS. 23A and 23B) and NFKB (FIGS. 23C and 23D), two reliable PD markers for the assessment of treated wounds or healing.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A composition comprising: one or more gelling agent comprising one or more cellulose agent or a derivative thereof and a recombinant Smad7 polypeptide, a fragment thereof, or fusion polypeptide thereof.

2. The composition according to claim 1, wherein the composition further comprises one or more protein stabilizers, one or more surfactants, one or more antimicrobials, one or more preservatives or a combination thereof.

3. The composition according to claim 1 or claim 2, wherein the gelling agent comprises a gelling agent having an average molecular weight (MW) of about 25,000 to about 1,500,000 Daltons.

4. The composition according to any one of claims 1-3, wherein the one or more gelling agent comprises one or more cellulose agent.

5. The composition according to any one of claims 1-4, wherein the one or more cellulose agent or derivative thereof comprises one or more of hydroxypropylmethylcellulose (HPMC), (HPC), hydroxyethylcellulose (HEC), hydroxypropylmethylcellulose phthalate (HPMCP), hydroxypropylmethylcellulose acetate succinate, or a combination thereof.

6. The composition according to claim 1, wherein the one or more cellulose agent or derivative thereof comprises HPC, HEC, HPMC or a combination thereof.

7. The composition according to claim 2, wherein the one or more protein stabilizers comprises one or more of succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate, sodium saccharin, or a combination thereof.

8. The composition according to claim 2, wherein the one or more surfactant comprises one or more of sodium lauryl sulfate, sodium decussate, Tween-20, Tween-60, Tween-80, triacetin, vitamin E TPGS, a phospholipid, a lecithin, a phosphatidyl choline, a phosphatidylethanolamine, a phosphatidylglycerol, sorbitan monooleate, polyoxyethylene sorbitan monooleate, a polysorbate, a polaxomer, bile salt, glyceryl monostearate, a copolymer of ethylene oxide and propylene oxide, polyoxyethylene hydrogenated castor oil, polyoxyethylene alkylether, octoxynol 10, octoxynol 40, or a combination thereof.

9. The composition according to claim 2, wherein the one or more preservative comprises one or more of benzalkonium chloride, chlorobutanol, thimerosol, chloroxylenol, chlorhexidine, phenoxyethanol, benzyl alcohol, phenethyl alcohol, polyquaterniaum-1, diazolidinyl urea, iodopropynyl butylcarbamate, chloromethylisotiazolinone, methylisothiazolinone, vitamin E, vitamin E derivative, vitamin E acetate, vitamin C, butylated hydroxytoluene, butylparaben, ethylparaben, methylparaben, propylparaben, isobutylparaben, phenoxyethanol, ethylparaben, propylparaben, utylparaben, or a combination thereof.

10. The composition according to any one of claim 1 or 3-8, wherein the composition is free of preservatives.

11. The composition according to any one of claims 1-10, wherein the recombinant Smad7, fragment thereof, or fusion polypeptide thereof comprises at least 85% identity to an amino acid sequence represented by SEQ ID NO: 1.

12. The composition according to claim 1-10, wherein the recombinant Smad7, fragment thereof, or fusion polypeptide thereof comprises at least 85% identity to an amino acid sequence of residue 203-residue 426 of a human Smad7 or the amino acid sequence represented by SEQ ID NO: 3.

13. The composition according to any one of claims 1-12, wherein the recombinant Smad7 fusion polypeptide or Smad7 fragment fusion polypeptide comprises at least one protein transduction domain.

14. The composition according to any one of claims 1-13, wherein the recombinant Smad7 fusion polypeptide or Smad7 fragment fusion polypeptide comprises at least one epitope tag.

15. The composition according to any one of claims 1-14, wherein the pH of the composition is about pH 7.0 to about pH 9.0.

16. The composition according to any one of claims 1-15, wherein the composition further comprises a salt.

17. The composition according to any one of claims 1-16, wherein the composition further comprises at least one of sodium phosphate and sodium chloride.

18. The composition according to any one of claims 1-17, wherein the composition is formulated for topical administration.

19. The composition according to any one of claims 1-17, wherein the composition is formulated for intradermal administration.

20. The composition according to any one of claims 1-17, wherein the composition is formulated for administration to the oral mucosa.

21. The composition according to any one of claims 1-17, wherein the composition is an oral gel.

22. The composition according to any one of claims 1-17, wherein the composition is a gel for topical administration to the skin.

23. A method of reducing the risk of, preventing, treating, and/or ameliorating a health condition in a subject, wherein the method comprises administering a composition according to any one of claims 1-22 to the subject having or suspected of developing the health condition, wherein the health condition includes excess inflammation or side effect of radiation therapy or chemotherapy.

24. The method according to claim 23, wherein the composition comprises a topically formulated composition and administering the topically formulated composition to an affected area of the subject.

25. The method according to claim 23 or 24, wherein the condition is an acute or a chronic wound, a wound at risk for scar formation, a chronically infected tissue, oral mucositis, radiation dermatitis or radiodermatitis, psoriasis, atopic dermatitis, contact dermatitis, allergic dermatitis, autoimmune related oral ulcer, periodontal inflammation, or a combination thereof.

26. The method according to claim 24, wherein the condition is due to side effects of a therapeutic agent used to treat cancer in the subject.

27. The method according to any one of claims 23-26, wherein the subject is human or other mammalian subject.

28. The method according to any one of claims 23-27, wherein the subject is undergoing cancer therapy and at risk for developing oral mucositis, radiodermatitis or both, and wherein the Smad7 polypeptide is Tat-PYC-Smad7 fusion polypeptide formulated in an oral formulation and administered daily.

29. The method according to any one of claims 23-28, wherein the daily dose administered to the subject is about 0.1 μg to about 50.0 μg per day for a predetermined period or until symptoms resolve.

30. The method according to any one of claims 23-29, wherein the subject has started cancer therapy and has clinical indicators of oral mucositis, radiodermatitis or both, and wherein administration of the composition reduces or prevents onset or progression of oral mucositis radiodermatitis or both.

31. The method according to any one of claims 23-30, wherein the subject is experiencing excess inflammation and the excess inflammation is reduced systemically.

32. A kit comprising one or more compositions according to claims 1-22, and at least one container.

33. The kit according to claim 32, further comprising a device for delivering the one or more compositions to a subject.
